(19) United States
(12) Patent Application Publication
(10) Pub. No.: US 2012/0004285 A1
(43) Pub. Date: Jan. 5, 2012

(54) ERBB-3 (HER3)-SELECTIVE COMBINATION THERAPY

(75) Inventors: Baisong Liao, Highland Park, NJ (US); Yixian Zhang, Piscataway, NJ (US)

(73) Assignee: ENZON PHARMACEUTICALS, INC., Bridgewater, NJ (US)

(21) Appl. No.: 13/127,270

(22) PCT Filed: Nov. 5, 2009
(86) PCT No.: PCT/US09/63357

§ 371 Date: Jul. 5, 2011

Related U.S. Application Data
(60) Provisional application No. 61/112,549, filed on Nov. 7, 2008.

(51) Int. Cl. A61K 31/7115; A61P 35/00; A61P 35/02; C12N 5/09

(52) U.S. Cl. 514/44 R; 435/325

(57) ABSTRACT

The invention relates to pharmaceutical compositions for and methods of treatment with HER3-targeted combination therapy. The invention relates to pharmaceutical compositions comprising an oligomer which targets HER3 (and optionally one or more of HER2 and EGFR) mRNA in a cell, leading to reduced expression of HER3 and optionally HER2 and/or EGFR, and a small molecule protein tyrosine kinase inhibitor of one or more receptor tyrosine kinases, leading to inhibition of signaling and/or internalization of receptor dimers into the cell. The combination therapy is beneficial for a range of medical disorders, such hyperproliferative disorders (e.g., cancer). The invention provides methods of treating hyperproliferative disorders with a combination of an oligomer and a protein tyrosine kinase inhibitor.

### CROSS-REFERENCE TO RELATED APPLICATIONS

This application claims priority to U.S. provisional patent application Ser. No. 61/112,549 filed Nov. 7, 2008, which is incorporated by reference herein in its entirety.

### FIELD OF THE INVENTION

The invention relates to methods of down-regulating the expression and/or activity of HER3 (and optionally of one or more of EGFR and HER2) in a cell, comprising administering to the cell an effective amount of an oligomeric compound (oligomer) that targets HER3 mRNA in a cell and an effective amount of a protein tyrosine kinase (PTK) inhibitor, or a pharmaceutically acceptable derivative thereof. The invention further relates to methods of treating a disease comprising administering to a patient in need thereof an effective amount of an oligomer that targets HER3 mRNA in a cell and an effective amount of a PTK inhibitor, or a pharmaceutically acceptable derivative thereof. The invention further relates to pharmaceutical compositions comprising an effective amount of an oligomer that targets HER3 mRNA and an effective amount of a PTK inhibitor, or a pharmaceutically acceptable derivative thereof, in a pharmaceutically acceptable excipient. The compositions are useful for down-regulating the expression and/or activity of HER3 (and optionally of one or more of EGFR and HER2) and for treating various diseases such as cancer.

The invention provides for use of a locked nucleic acid (“LNA”) oligomer targeting HER3, such as one or more of the oligomers described herein, for the preparation of a medicament, wherein the medicament is for the use in the treatment of cancer in combination with a protein tyrosine kinase inhibitor. The invention provides for a medicament comprising an LNA oligomer targeting HER3, such as one or more of the oligomers described herein, wherein the medicament is for the use in the treatment of cancer in combination with a protein tyrosine kinase inhibitor.

### 1.1. BACKGROUND

HER3 is a member of the ErbB family of receptor tyrosine kinases, which includes four different receptors: ErbB-1 (EGFR, HER1), ErbB-2 (neu, HER2), ErbB-3 (HER3) and ErbB-4 (HER4) (Yarden et al., Nat. Rev. Mol. Cell. Biol, 2001, 2(2):127-137). The receptor proteins of this family are composed of an extracellular ligand-binding domain, a single hydrophobic transmembrane domain and a cytoplasmic tyrosine kinase-containing domain. There are at least 12 growth factors in the EGF family that bind to one or more of the ErbB receptors and effect receptor homo- or hetero-dimerization. Dimerization triggers internalization and recycling of the ligand-bound receptor (or its degradation), as well as downstream intracellular signaling pathways that regulate, inter cilia, cell survival, apoptosis and proliferative activity. HER3 (ErbB3) is understood by those skilled in the art to lack tyrosine kinase activity.

EGFR, HER2 and recently HER3 have been associated with tumor formation. Recent studies have shown that EGFR is over expressed in a number of malignant human tissues when compared to their normal tissue counterparts. A high incidence of over-expression, amplification, deletion and structural rearrangement of the gene coiling for EGFR has been found in tumors of the breast, lung, ovaries and kidney. Amplification of the EGFR gene in glioblastoma multiforme tumors is one of the most consistent genetic alterations known. EGFR overexpression has also been noted in many non-small cell lung carcinomas. Elevated levels of HER3 mRNA have been detected in human mammary carcinomas.

Conventional chemotherapy regimens, which are directed toward cellular proteins or other macromolecules and lead to apoptosis, typically do not discriminate between fast-dividing tumor cells and rapidly dividing normal cells. The death of normal cells such as bone marrow cells and cells of the gastrointestinal tract, leads to toxic side effects. In addition, tumor responses from cytotoxic chemotherapy are unpredictable.

Recently, several protein tyrosine kinase inhibitors have been explored as selective therapies for certain cancers in which protein tyrosine kinase expression is dysregulated. However, the efficacy of such therapies is limited because many cancers do not respond to protein tyrosine kinase inhibitor therapies or a resistance to the inhibitors develops over time. Arora et al. (2005) J. Pharmacol. and Exp. Therapies 315(3):971-971-979.

There is a need for cancer therapies that are targeted to tumor cells, that are more effective and less toxic than conventional chemotherapy, and that have a higher response rate than currently available selective therapies.

### 1.2. SUMMARY

In certain embodiments, the invention relates to a pharmaceutical composition comprising: (a) an oligomer consisting of 10 to 50 contiguous monomers wherein adjacent monomers are covalently linked by a phosphate group or a phosphorothioate group, wherein the oligomer comprises a first region of at least 10 contiguous monomers; wherein at least one monomer of the first region is a nucleoside analog; wherein the sequence of the first region is at least 80% identical to the reverse complement of the best-aligned target region of a mammalian HER3 gene or a mammalian HER3 mRNA; (b) a protein tyrosine kinase inhibitor; and (c) a pharmaceutically acceptable excipient.

In various embodiments, the pharmaceutical composition comprises an oligomer consisting of the sequence shown in SEQ ID NO: 180 and the protein tyrosine kinase inhibitor gefitinib.

In other embodiments, the pharmaceutical composition comprises: (a) a conjugate of an oligomer consisting of 10 to 50 contiguous monomers wherein adjacent monomers are covalently linked by a phosphate group or a phosphorothioate group, wherein the oligomer comprises a first region of at least 10 contiguous monomers; wherein at least one monomer of the first region is a nucleoside analog; wherein the sequence of the first region is at least 80% identical to the reverse complement of the best-aligned target region of a mammalian HER3 gene or a mammalian HER3 mRNA; (b) a protein tyrosine kinase inhibitor; and (c) a pharmaceutically acceptable excipient.

The invention further relates to a method of inhibiting the proliferation of a mammalian cell, comprising contacting the cell with: (a) an effective amount of an oligomer consisting of 10 to 50 contiguous monomers wherein adjacent monomers are covalently linked by a phosphate group or a phosphorothioate group, wherein the oligomer comprises a first region of at least 10 contiguous monomers; wherein at least one monomer of the first region is a nucleoside analog; and wherein the sequence of the first region is at least 80% identical to the reverse complement of the best-aligned target region of a mammalian HER3 gene or a mammalian HER3 mRNA; and (b) an effective amount of a protein tyrosine kinase inhibitor.

In various embodiments, the method of inhibiting the proliferation of a mammalian cell comprises contacting the cell with an effective amount of an oligomer consisting of the sequence shown in SEQ ID NO: 180 and an effective amount of gefitinib.

In some embodiments, the invention encompasses methods of inhibiting the proliferation of cells in the body of a mammal, comprising contacting a mammalian tissue with: (a) an effective amount of an oligomer consisting of 10 to 50 contiguous monomers wherein adjacent monomers are covalently linked by a phosphate group or a phosphorothioate group, wherein the oligomer comprises a first region of at least 10 contiguous monomers; wherein at least one monomer of the first region is a nucleoside analog; and wherein the sequence of the first region is at least 80% identical to the reverse complement of the best-aligned target region of a mammalian HER3 gene or a mammalian HER3 mRNA; and (b) an effective amount of a protein tyrosine kinase inhibitor.

In certain embodiments, the method of inhibiting the proliferation of cells in the body of a mammal comprises contacting a mammalian tissue with an effective amount of an oligomer consisting of the sequence shown in SEQ ID NO: 180 and an effective amount of gefitinib.

In various embodiments, the method of inhibiting the proliferation of cells in the body of a mammal comprises contacting a mammalian tissue with: (a) an effective amount of a conjugate of an oligomer consisting of 10 to 50 contiguous monomers wherein adjacent monomers are covalently linked by a phosphate group or a phosphorothioate group, wherein the oligomer comprises a first region of at least 10 contiguous monomers; wherein at least one monomer of the first region is a nucleoside analog; and wherein the sequence of the first region is at least 80% identical to the reverse complement of the best-aligned target region of a mammalian HER3 gene or a mammalian HER3 mRNA; and (b) an effective amount of a protein tyrosine kinase.

The invention further encompasses a method of treating cancer in a mammal, comprising administering to the mammal: (a) an effective amount of an oligomer consisting of 10 to 50 contiguous monomers wherein adjacent monomers are covalently linked by a phosphate group or a phosphorothioate group, wherein the oligomer comprises a first region of at least 10 contiguous monomers; wherein at least one monomer of the first region is a nucleoside analog; wherein the sequence of the first region is at least 80% identical to the reverse complement of the best-aligned target region of a mammalian HER3 gene or a mammalian HER3 mRNA; and (b) an effective amount of a protein tyrosine kinase inhibitor.

In certain embodiments, the method of treating cancer in a mammal comprises administering to the mammal an effective amount of an oligomer consisting of the sequence shown in SEQ ID NO: 180 and an effective amount of gefitinib.

In various embodiments, the cancer is selected from the group consisting of lung cancer, prostate cancer, breast cancer, ovarian cancer, colon cancer, epithelial carcinoma, and stomach cancer.

In further embodiments, the invention encompasses a method of treating cancer in a mammal, comprising administering to the mammal: (a) an effective amount of a conjugate of an oligomer consisting of 10 to 50 contiguous monomers wherein adjacent monomers are covalently linked by a phosphate group or a phosphorothioate group, wherein the oligomer comprises a first region of at least 10 contiguous monomers; wherein at least one monomer of the first region is a nucleoside analog; wherein the sequence of the first region is at least 80% identical to the reverse complement of the best-aligned target region of a mammalian HER3 gene or a mammalian HER3 mRNA; and (b) an effective amount of a protein tyrosine kinase inhibitor.

One embodiment of the invention provides the use of

(a.) an oligomer consisting of 10 to 50 contiguous monomers wherein adjacent monomers are covalently linked by a phosphate group or a phosphorothioate group,
wherein said oligomer comprises a first region of at least 10 contiguous monomers that is at least 80% identical to the sequence of a region of at least 10 contiguous monomers present in a compound selected from the group consisting of

(SEQ ID NO: 169)
5′-Gₛ^{Me}CₛTₛcₛcₛaₛgₛaₛcₛaₛtₛcₛaₛ^{Me}CₛTₛ^{Me}C-3;
and
(SEQ ID NO: 180)
5′-TₛAₛGₛcₛcₛtₛgₛtₛcₛaₛcₛtₛtₛ^{Me}CₛTₛ^{Me}C-3,

wherein uppercase letters denote beta-D-oxy-LNA monomers and lowercase letters denote DNA monomers, the subscript “s” denotes a phosphorothioate linkage, and ^{Me}C denotes a beta-D-oxy-LNA monomer containing a 5-methylcytosine base, and
wherein at least one monomer of said first region is a nucleoside analogue,

said oligomer being an antisense inhibitor or HER3; and

(b.) a protein tyrosine kinase inhibitor of EGFR (HER1) such as gefitinib, erlotinib, lapatinib and canertinib and/or a protein tyrosine kinase inhibitor of a VEGFR family member, such as VEGFR2 and VEGFR3, such as sorafenib,

in combination for the treatment of a cancer in a mammal.

In one variation of the embodiment, the sequence of the first region is identical to the sequence of a region of at least 10 contiguous monomers present in 5′-Gₛ^{Me}CₛTₛcₛcₛaₛgₛaₛcₛaₛtₛcₛaₛ^{Me}CₛTₛ^{Me}C-3′ (SEQ ID NO: 169) or 5′-TₛAₛGₛcₛcₛtₛgₛtₛcₛaₛcₛtₛtₛ^{Me}CₛTₛ^{Me}C-3′ (SEQ ID NO: 180). In another variation of the embodiment, the oligomer is 5′-Gₛ^{Me}CₛTₛcₛcₛaₛgₛaₛcₛaₛtₛcₛaₛ^{Me}CₛTₛ^{Me}C-3′ (SEQ ID NO: 169) or 5′-TₛAₛGₛcₛcₛtₛgₛtₛcₛaₛcₛtₛtₛ^{Me}CₛTₛ^{Me}C-3′ (SEQ ID NO: 180), which are antisense oligomer inhibitors of HER3. Method-of-treatment embodiments that correspond to these uses are also provided by the invention. Said method embodiments include the administration to a mammal, such as a human patient, in need of treatment for a cancer of the oligomer and the PKI inhibitor at or around the same lime.

### 1.3. BRIEF DESCRIPTION OF THE FIGURES

FIG. 1A-1C. FIGS. 1A and 1B show the anti-proliferative effects on A549 lung cancer cells of treatment with a combination of an oligomeric compound (having a sequence and design as set forth in SEQ ID NO: 180) and gefitinib. FIG. 1C demonstrates the inhibition of HER3 mRNA expression in A549 cells by the oligomeric compound having the sequence and design as set forth in SEQ ID NO: 180.

FIG. 2A-2C. FIGS. 2A and 2B show the anti-proliferative effects on H1993 prostate cancer cells of treatment with a combination of an oligomeric compound (having a sequence and design as set forth in SEQ ID NO: 180) and gefitinib. FIG. 2C demonstrates the inhibition of HER3 mRNA expression in H1993 cells by the oligomeric compound having the sequence and design as set forth in SEQ ID NO: 180.

FIG. 3A-3C. FIGS. 3A and 3B show the anti-proliferative effects on 15PC3 prostate cancer cells of treatment with a combination of an oligomeric compound (having a sequence and design as set forth in SEQ ID NO: 180) and gefitinib. FIG. 3C demonstrates the inhibition of HER3 mRNA expression in 15PC3 cells by the oligomeric compound having the sequence and design as set forth in SEQ ID NO: 180.

FIG. 4A-4C. FIGS. 4A and 4B show the anti-proliferative effects on DU145 prostate cancer cells of treatment with a combination of an oligomeric compound (having a sequence and design as set forth in SEQ ID NO: 180) and gefitinib. FIG. 4C demonstrates the inhibition of HER3 mRNA expression in DU145 cells by the oligomeric compound having the sequence and design as set forth in SEQ ID NO: 180.

FIG. 5A-5C. FIGS. 5A and 5B show the anti-proliferative effects on SKBR3 breast cancer cells of treatment with a combination of an oligomeric compound (having a sequence and design as set forth in SEQ ID NO: 180) and gefitinib. FIG. 5C demonstrates the inhibition of HER3 mRNA expression in SKBR3 cells by the oligomeric compound having the sequence and design as set forth in SEQ ID NO: 180.

FIG. 6A-6C. FIGS. 6A and 6B show the anti-proliferative effects on A431 human epithelial carcinoma cells of treatment with a combination of an oligomeric compound (having a sequence and design as set forth in SEQ ID NO: 180) and gefitinib. FIG. 6C demonstrates the inhibition of HER3 mRNA expression in A431 cells by the oligomeric compound having the sequence and design as set forth in SEQ ID NO: 180.

### 1.4. DETAILED DESCRIPTION

In certain embodiments, the invention provides compositions and methods for modulating the expression and/or activity of HER3 (and optionally one or more of EGFR and HER2). In particular, the invention provides for pharmaceutical compositions comprising an effective amount of an oligomer that specifically hybridizes under intracellular conditions to nucleic acids encoding HER3 (and optionally one or more of EGFR and HER2) and an effective amount of a protein tyrosine kinase inhibitor, or a pharmaceutically acceptable derivative thereof, in a pharmaceutically acceptable excipient.

In certain embodiments, the oligomer is present in the same composition as the protein tyrosine kinase inhibitor, or pharmaceutically acceptable derivative thereof. In various embodiments, the oligomer is present in a composition that is separate from the composition that comprises the protein tyrosine kinase inhibitor. In certain embodiments, the oligomer is present in a separate composition from the protein tyrosine kinase inhibitor composition, and the two compositions are packaged for use in combination.

In certain embodiments, the invention encompasses methods of treating or preventing a disorder, such as cancer, in a patient comprising administering to a patient in need thereof an effective amount of the pharmaceutical compositions of the invention.

### 1.5. PHARMACEUTICAL COMPOSITIONS

1.5.1. Oligomers

In a first aspect, oligomeric compounds (referred to herein as oligomers) for use in the pharmaceutical compositions and methods of the invention are useful, e.g., in modulating the function of nucleic acid molecules encoding mammalian HER3. In certain embodiments, the nucleic acid molecules encoding mammalian HER3 include nucleic acids having the base sequence shown in SEQ ID No: 197, and naturally occurring allelic variants thereof. The oligomers of the invention are composed of covalently linked monomers.

The term “monomer” includes both nucleosides and deoxynucleosides (collectively, “nucleosides”) that occur naturally in nucleic acids and that do not contain either modified sugars or modified nucleobases, i.e., compounds in which a ribose sugar or deoxyribose sugar is covalently bonded to a naturally-occurring, unmodified nucleobase (base) moiety (i.e., the purine and pyrimidine heterocycles adenine, guanine, cytosine, thymine or uracil) and “nucleoside analogs,” which are nucleosides that either do occur naturally in nucleic acids or do not occur naturally in nucleic acids, wherein either the sugar moiety is other than a ribose or a deoxyribose sugar (such as bicyclic sugars or 2′ modified sugars, such as 2′ substituted sugars), or the base moiety is modified (e.g., 5-methylcytosine), or both.

An “RNA monomer” is a nucleoside containing a ribose sugar and an unmodified nucleobase.

A “DNA monomer” is a nucleoside containing a deoxyribose sugar and an unmodified nucleobase.

A “Locked Nucleic Acid monomer,” “locked monomer,” or “LNA monomer” is a nucleoside analog having a bicyclic sugar, as further described herein below.

The terms “corresponding nucleoside analog” and “corresponding nucleoside” indicate that the base moiety in the nucleoside analog and the base moiety in the nucleoside are identical. For example, when the “nucleoside” contains a 2-deoxyribose sugar linked to an adenine, the “corresponding nucleoside analog” contains, for example, a modified sugar linked to an adenine base moiety.

The monomers of the oligomers described herein for use in the compositions and methods of the invention are coupled together via linkage groups. Suitably, each monomer is linked to the 3′ adjacent monomer via a linkage group.

The terms “linkage group” or “internucleoside linkage” mean a group capable of covalently coupling together two contiguous monomers. Specific examples include phosphate groups (forming a phosphodiester between adjacent nucleoside monomers) and phosphorothioate groups (forming a phosphorothioate linkage between adjacent nucleoside monomers).

Suitable linkage groups include those listed in WO 2007/031091, for example the linkage groups listed on the first paragraph of page 34 of WO 2007/031091 (hereby incorporated by reference).

In some embodiments, the linkage group is modified from its normal phosphodiester to one that is more resistant to nuclease attack, such as phosphorothioate or boranophosphate, which are cleavable by RNase H, permitting RNase-mediated antisense inhibition of expression of the target gene.

The terms “oligomer,” “oligomeric compound,” and “oligonucleotide” are used interchangeably in the context of the invention, and refer to a molecule formed by covalent linkage of two or more contiguous monomers by, for example, a phosphate group (forming a phosphodiester linkage between nucleosides) or a phosphorothioate group (forming a phosphorothioate linkage between nucleosides). The oligomer comprises or consists of 10-50 monomers, such as 10-30 monomers.

In some embodiments, an oligomer comprises nucleosides, or nucleoside analogs, or mixtures thereof as referred to herein. An “LNA oligomer” or “LNA oligonucleotide” refers to an oligonucleotide containing one or more LNA monomers, as defined below in Section 6.1.2.

Nucleoside analogs that are optionally included within oligomers may function similarly to corresponding nucleosides, or may have specific improved functions. Oligomers wherein some or all of the monomers are nucleoside analogs are often preferred over native forms because of, e.g., their increased ability to penetrate a cell membrane, good resistance to extra- and/or intracellular nucleases, and high affinity and specificity for the nucleic acid target. LNA monomers are particularly preferred.

In various embodiments, one or more nucleoside analogs present within the oligomer are “silent” or “equivalent” in function to the corresponding natural nucleoside, i.e., have no functional effect on the way the oligomer functions to inhibit target gene expression. Such “equivalent” nucleoside analogs are nevertheless useful if, for example, they are easier or cheaper to manufacture, or are more stable under storage or manufacturing conditions, or can incorporate a tag or label. Typically, however, the analogs will have a functional effect on the way in which the oligomer functions to inhibit expression, e.g., by producing increased binding affinity to the target region of the target nucleic acid and/or increased resistance to intracellular nucleases and/or increased ease of transport into the cell.

In various embodiments, oligomers according to the invention comprise nucleoside monomers and at least one nucleoside analog monomer, such as an LNA monomer, or other nucleoside analog monomers.

The term “at least one” comprises the integers larger than or equal to 1, such as 1, 2, 3, 4, 5, 6, 7, 8, 9, 10, 11, 12, 13, 14, 15, 16, 17, 18, 19, 20 and so forth. In various embodiments, such as when referring to the nucleic acid or protein targets of the compounds of the invention, the term “at least one” includes the terms “at least two” and “at least three” and “at least four.” Likewise, in some embodiments, the term “at least two” comprises the terms “at least three” and “at least four.”

In some embodiments, the oligomer consists of 10-50 contiguous monomers, such as 10, 11, 12, 13, 14, 15, 16, 17, 18, 19, 20, 21, 22, 23, 24, 25, 26, 27, 28, 29 or 30 contiguous monomers.

In some embodiments, the oligomer consists of 10-25 monomers, or of 10-16 monomers, or of 12-16 monomers.

In various embodiments, the oligomers comprise 10-25 contiguous monomers, 10-24 contiguous monomers, 12-25 or 12-24 or 10-22 contiguous monomers, such as 12-18 contiguous monomers, such as 13-17 or 12-16 contiguous monomers, such as 13, 14, 15, 16 contiguous monomers.

In various embodiments, the oligomers comprise 10-22 contiguous monomers, or 10-18, such as 12-18 or 13-17 or 12-16, such as 13, 14, 15 or 16 contiguous monomers.

In some embodiments, the oligomers comprise 10-16 or 12-16 or 12-14 contiguous monomers. In other embodiments, the oligomers comprise 14-18 or 14-16 contiguous monomers.

In various embodiments, the oligomers comprise 10, 11, 12, 13, or 14 contiguous monomers.

In various embodiments, the oligomers for use in pharmaceutical compositions and methods of the invention consist of no more than 22 contiguous monomers, such as no more than 20 contiguous monomers, such as no more than 18 contiguous monomers, such as 15, 16 or 17 contiguous monomers. In certain embodiments, the oligomer of the invention comprises fewer than 20 contiguous monomers.

In various embodiments, the oligomer of the invention does not comprise RNA monomers.

In various embodiments, the oligomers are linear molecules or are linear as synthesized. The oligomer is, in such embodiments, a single stranded molecule, and typically does not comprise a short region of, for example, at least 3, 4 or 5 contiguous monomers, which are complementary to another region within the same oligomer such that the oligomer forms an internal duplex. In various embodiments, the oligomer is not substantially double-stranded, i.e., is not a siRNA.

In some embodiments, the oligomers consist of a contiguous stretch of monomers, the sequence of which is identified by a SEQ ID NO. disclosed herein (see, e.g., Table 1). In other embodiments, the oligomers comprise a first region, the region consisting of a contiguous stretch of monomers, and one or more additional regions which consist of at least one additional monomer. In some embodiments, the sequence of the first region is identified by a SEQ ID NO. disclosed herein.

1.5.2. Locked Nucleic Acid (LNA) Monomers

The term “LNA monomer” refers to a nucleoside analog containing a bicyclic sugar (an “LNA sugar”). The terms “LNA oligonucleotide” and “LNA oligomer” refer to an oligomer containing one or more LNA monomers.

In certain embodiments, the LNA used in the oligonucleotide compounds used in the compositions and methods of the invention has the structure of the general formula I:

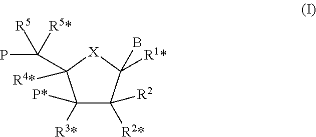

wherein X is selected from —O—, —S—, —N(R^{N}*)—, —C(R⁶R⁶*)—;

B is selected from hydrogen, optionally substituted C₁₋₄-alkoxy, optionally substituted C₁₋₄-alkyl, optionally substituted C₁₋₄-acyloxy, nucleobases, DNA intercalators, photochemically active groups, thermochemically active groups, chelating groups, reporter groups, and ligands;

P designates the radical position for an internucleoside linkage to a succeeding monomer, or a 5′-terminal group, such internucleoside linkage or 5′-terminal group optionally including the substituent R⁵ or equally applicable the substituent R⁵*;

P* designates an internucleoside linkage to a preceding monomer, or a 3′-terminal group;

R⁴* and R²* together designate a biradical consisting of 1-4 groups/atoms selected from —C(R^{a}R^{b})—, —C(R^{a})═C(R^{b})—, —C(R^{a})═N—, —O—, —Si(R^{a})₂—, —S—, —SO₂—, —N(R^{a})—, and >C═Z,
wherein Z is selected from —O—, —S—, and —N(R^{a})—, and R^{a} and R^{b} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂), and

each of the substituents R¹*, R², R³*, R⁵, R⁵*, R⁶ and R⁶*, if present is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents together may designate oxo, thioxo, imino, or optionally substituted methylene, or together may form a spiro biradical consisting of a 1-5 carbon atom(s) alkylene chain which is optionally interrupted and/or terminated by one or more heteroatoms/groups selected from —O—, —S—, and —(NR^{N})— where R^{N} is selected from hydrogen and C₁₋₄-alkyl, and where two adjacent (non-geminal) substituents may designate an additional bond resulting in a double bond; and R^{N}*, when present and not involved in a biradical, is selected from hydrogen and C₁₋₄-alkyl; and basic salts and acid addition salts thereof;

In certain embodiments, R^{5*} is selected from H, —CH₃, —CH₂—CH₃, —CH₂—O—CH₃, and —CH—CH₂.

In various embodiments, R^{4*} and R^{2*} together designate a biradical selected from —C(R^{a}R^{b})—O—, —C(R^{a}R^{b})—C(R^{c}R^{d})—O—, —C(R^{a}R^{b})—C(R^{c}R^{d})—C(R^{e}R^{f})—O—, —C(R^{a}R^{b})—O—C(R^{c}R^{d})—, —C(R^{a}R^{b})—O—C(R^{c}R^{d})—O—, —C(R^{a}R^{b})—C(R^{c}R^{d})—, —C(R^{a}R^{b})—C(R^{c}R^{d})—C(R^{e}R^{f})—, —C(R^{a})═C(R^{b})—C(R^{c}R^{d})—, —C(R^{a}R^{b})—N(R_{c})—, —C(R^{a}R^{b})—C(R^{c}R^{d})—N(R^{e})—, —C(R^{a}R^{b})—N(R^{c})—O—, and —C(R^{a}R^{b})—S—, —C(R^{a}R^{b})—C(R^{c}R_{d})—S— herein R^{a}, R^{e}, and R^{f} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted and where two geminal substituents R^{a} and R^{b} together may designate optionally substituted methylene (═CH₂),

In further embodiments R^{4*} and R^{2*} together designate a biradical selected from —CH₂—O—, —CH₂—S—, —CH₂—N(CH₃)—, —CH₂—CH₂—O—, —CH₂—CH(CH₃)—, —CH₂—CH₂—S—, —CH₂—CH₂—NH—, —CH₂—CH₂—CH₂—, —CH₂—CH₂—CH₂—O—, —CH₂—CH₂—CH(CH₃)—, —CH═CH—CH₂—, —CH₂—O—CH₂—O—, —CH₂—NH—O—, —CH₂—N(CH₃)—O—, —CH₂—O—CH₂—, —CH(CH₃)—O—, —CH(CH₂—O—CH₃)—O—.

For all chiral centers, asymmetric groups may be found in either R or S orientation.

In various embodiments, the LNA monomer used in the oligomers comprises at least one LNA monomer according formula (II) or formula (III):

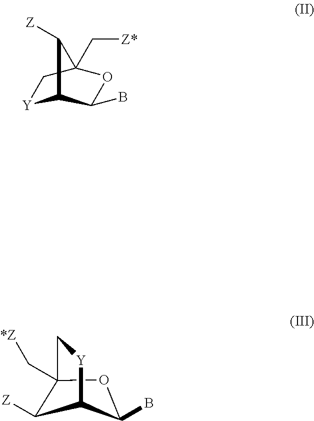

wherein Y is —O—, —O—CH₂—, —S—, —NH—, or N(R^{H}); Z and Z* are independently selected among an internucleoside linkage, a terminal group or a protecting group; B constitutes an unmodified base moiety or a modified base moiety that either occurs naturally in nucleic acids or does not occur naturally in nucleic acids, and R^{H} is selected from hydrogen and C₁₋₄-alkyl.

LNA monomers for use in various embodiments of the invention are shown in formulas (IV)-(VIII) below:

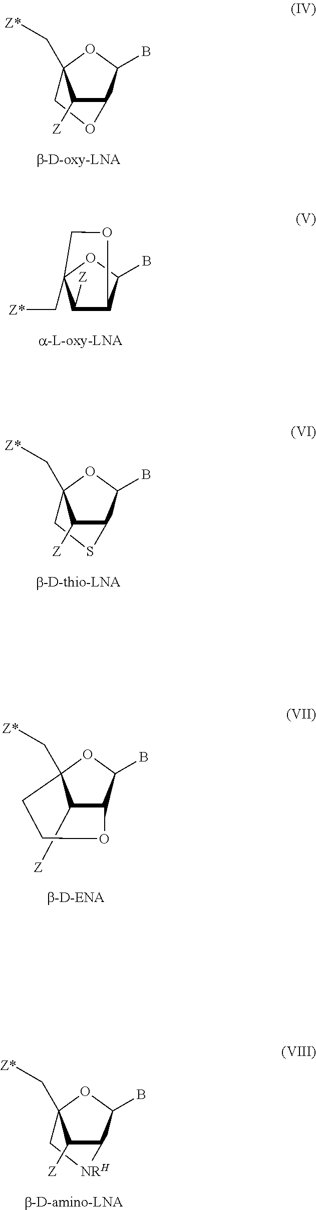

The term “thio-LNA” refers to an LNA monomer in which Y in formula (II) above is selected from S or —CH₂—S—. Thio-LNA can be in either the beta-D or the alpha-L configuration.

The term “amino-LNA” refers to an LNA monomer in which Y in formula (II) above is selected from —N(H)—, N(R)—, CH₂—N(H)—, and —CH₂—N(R)— where R is selected from hydrogen and C₁₋₄-alkyl. Amino-LNA can be in either the beta-D or the alpha-L configuration.

The term “oxy-LNA” refers to an LNA monomer in which Y in formula (II) above represents —O— or —CH₂—O—. Oxy-LNA can be in either the beta-D or the alpha-L configuration.

The term “ENA” refers to an LNA monomer in which Y in the formula (II) above is —CH₂—O— (where the oxygen atom of —CH₂—O— is attached to the 2′-position relative to the base B).

In certain embodiments, the LNA monomer is selected from a beta-D-oxy-LNA monomer, an alpha-L-oxy-LNA monomer, a beta-D-amino-LNA monomer and a beta-D-thio-LNA monomer, in particular a beta-D-oxy-LNA monomer.

In the present context, the term “C₁₋₄-alkyl” means a linear or branched saturated hydrocarbon chain wherein the chain has from one to four carbon atoms, such as methyl, ethyl, n-propyl, isopropyl, n-butyl, isobutyl, sec-butyl and tert-butyl.

Locked nucleic acid (LNA)-containing oligomers represent a new generation of antisense oligomers. Unlike previous oligonucleotides, nucleoside LNA monomers in LNA oligomers have an engineered O2′- to C4′-linkage within the sugar (see formulas IV-VIII above). This stabilizes, or “locks” the ribose in the 3′-endo structural conformation that is favored for RNA binding. Hence, LNA oligomers have an exceptionally high binding affinity for RNA compared with conventional DNA oligomers. In addition, the LNA modification substantially improves nuclease resistance and permits reduction in oligonucleotide length (See, e.g., Vester B, et al. LNA (locked nucleic acid): high-affinity targeting of complementary RNA and DNA. Biochemistry. 2004 Oct. 26; 43(42):13233-41; Lauritsen A, et al. Methylphosphonate LNA: a locked nucleic acid with a methylphosphonate linkage. Bioorg Med Chem. Lett. 2003 Jan. 20; 13(2):253-6).

LNA monomers and oligonucleotides comprising LNA monomers can be obtained by any method known in the art. In certain embodiments, LNA monomers and LNA oligonucleotides can be obtained by the procedures disclosed in PCT Publication No. WO 07/031,081, and references cited therein.

1.5.3. Other Nucleoside Analog Monomers and Linkages

In various embodiments, at least one of the monomers present in the oligomer is a nucleoside analog that contains a modified base, such as a base selected from 5-methylcytosine, isocytosine, pseudoisocytosine, 5-bromouracil, 5-propynyluracil, 6-aminopurine, 2-aminopurine, inosine, diaminopurine, 2-chloro-6-aminopurine, xanthine and hypoxanthine, and/or a modified sugar, e.g., a sugar moiety modified to provide a 2′-substituent group, such as 2′-O-alkyl-ribose sugars, 2′-amino-deoxyribose sugars, 2′-fluoro-deoxyribose sugars, and 2′-O-methoxyethyl-ribose sugars (2′MOE), or an LNA sugar as described above, or arabinose sugars (“ANA monomers”), or 2′-fluoro-arabinose sugars, or d-arabino-hexitol sugars (“HNA monomers”).

Specific examples of nucleoside analogs useful in the oligomers described herein are described in e.g. Freier & Altmann; Nucl. Acid Res., 1997, 25, 4429-4443 and Uhlmann; Curr. Opinion in Drug Development, 2000, 3(2), 293-213 or described in or referenced in WO 2007/031091, incorporated herein by reference in its entirety.

In various embodiments, incorporation of affinity-enhancing nucleoside analogs (i.e., nucleoside analogs that raise the duplex stability (Tm) of the oligomer/target region duplex) in the oligomer, such as LNA monomers or monomers containing 2′-substituted sugars, or incorporation of modified linkage groups provides increased nuclease resistance. In various embodiments, incorporation of such affinity-enhancing nucleoside analogs allows the size of the oligomer to be reduced, and allows for greater sequence specificity for shorter oligomers. It will be recognized that when referring to a particular oligomer base sequence, in certain embodiments the oligomers comprise a corresponding affinity-enhancing nucleoside analog, such as a corresponding LNA monomer or other corresponding nucleoside analog.

Oligonucleotides comprising nucleoside and/or nucleoside analog monomers can be synthesized by any method known in the art. In some embodiments, oligonucleotides for use in the methods and compositions of the invention can be synthesized using an automated DNA synthesizer using standard phosphoramidite chemistry with oxidation by iodine. β-cyanoethyldiisopropyl-phosphoramidites can be purchased from Applied Biosystems (Foster City, Calif.). Modified monomers for use in making the oligomeric compounds used in the compositions and methods of the invention can be obtained by any method known in the art, such as those set forth in Jones R. and Herdewijn P., Current Protocols in Nucleic Acid Chemistry (John Wiley & Sons, Inc., eds. 2008).

In some embodiments, the linkage between at least 2 contiguous monomers of the oligomer is other than a phosphodiester linkage.

In certain embodiments, the oligomer includes at least one monomer that has a modified base, at least one monomer (which may be the same monomer) that has a modified sugar, and at least one inter-monomer linkage that is non-naturally occurring.

1.5.4. Gapmer Design

In certain embodiments, the oligomer of the invention is a gapmer.

A “gapmer” is an oligomer which comprises a contiguous stretch of monomers capable of recruiting an RNAse (e.g. RNAseH) as further described herein below, such as a region of at least 6 or 7 DNA monomers, referred to herein as region B, wherein region B is flanked both on its 5′ and 3′ ends by regions respectively referred to as regions A and C, each of regions A and C comprising nucleoside analogs, such as affinity-enhancing nucleoside analogs, such as 1-6 affinity-enhancing analogs, for example LNA nucleotides.

In certain embodiments, the nucleoside analogs present in regions A and C comprise modified sugar moieties, as described above, and all nucleoside analogs in the oligomer or in a region thereof comprise the same modified sugar moiety. In various embodiments, the nucleoside analogs contain 2′-MOE sugars, 2′-fluoro-deoxyribose sugars or LNA sugars. The nucleoside analogs of the oligomer can be independently selected from these three types. In certain oligomer embodiments containing nucleoside analogs, at least one of the nucleoside analogs contains a 2′-MOE-sugar. In various embodiments, at least 2, 3, 4, 5, 6, 7, 8, 9 or 10 nucleoside analogs in the oligomer contain 2′-MOE-ribose sugars. In certain embodiments, at least one of the nucleoside analogs contains a 2′-fluoro-deoxyribose sugar. In various embodiments, at least 2, 3, 4, 5, 6, 7, 8, 9 or 10 nucleoside analogs in the oligomer contain 2′-fluoro-deoxyribose sugars.

Typically, the gapmer comprises regions, from 5′ to 3′, A-B-C, or optionally A-B-C-D or D-A-B-C, wherein: region A comprises at least one nucleoside analog, such as at least one LNA monomer, such as 1-6 nucleoside analogs, such as LNA monomers; and region B comprises at least five contiguous monomers which are capable of recruiting RNAse (when formed in a duplex with a complementary target region of the target RNA molecule, such as the mRNA target), such as DNA monomers; and region C consists of or comprises at least one nucleoside analog, such as at least one LNA monomer, such as 1-6 nucleoside analogs, such as LNA monomers, and; region D, when present, comprises 1, 2 or 3 monomers, such as DNA monomers.

In various embodiments, region A consists of 1, 2, 3, 4, 5 or 6 nucleoside analogs, such as LNA monomers, such as 2-5 nucleoside analogs, such as 2-5 LNA monomers, such as 3 or 4 nucleoside analogs, such as 3 or 4 LNA monomers; and/or region C consists of 1, 2, 3, 4, 5 or 6 nucleoside analogs, such as LNA monomers, such as 2-5 nucleoside analogs, such as 2-5 LNA monomers, such as 3 or 4 nucleoside analogs, such as 3 or 4 LNA monomers. In some embodiments all the nucleoside analogs are LNA monomers.

In certain embodiments, region B comprises 5, 6, 7, 8, 9, 10, 11 or 12 contiguous monomers capable of recruiting RNAse, or 6-10, or 7-9, such as 8 contiguous monomers which are capable of recruiting RNAse. In certain embodiments, region B comprises at least one DNA monomer, such as 1-12 DNA monomers, or 4-12 DNA monomers, or 6-10 DNA monomers, such as 7-10 DNA monomers, or 8, 9 or 10 DNA monomers.

In certain embodiments, region A consists of 3 or 4 nucleoside analogs, such as LNA monomers, region B consists of 7, 8, 9 or 10 DNA monomers, and region C consists of 3 or 4 nucleoside analogs, such as LNA monomers. Such designs include (A-B-C) 3-10-3, 3-10-4, 4-10-3, 3-9-3, 3-9-4, 4-9-3, 3-8-3, 3-8-4, 4-8-3, 3-7-3, 3-7-4, 4-7-3, and may further include region D, which may have one or 2 monomers, such as DNA monomers.

In certain embodiments, the oligomer consists of 10, 11, 12, 13 or 14 contiguous monomers, wherein the regions of the oligomer have the pattern (5′-3′), A-B-C, or optionally A-B-C-D or D-A-B-C, wherein region A consists of 1, 2 or 3 nucleoside analogs, such as LNA monomers; region B consists of 7, 8 or 9 contiguous monomers which are capable of recruiting RNAse when formed in a duplex with a complementary RNA molecule (such as a mRNA target); and region C consists of 1, 2 or 3 nucleoside analogs, such as LNA monomers. When present, region D consists of a single DNA monomer.

In certain embodiments, region A consists of 1 LNA monomer. In certain embodiments, region A consists of 2 LNA monomers. In certain embodiments, region A consists of 3 LNA monomers. In certain embodiments, region C consists of 1 LNA monomer. In certain embodiments, region C consists of 2 LNA monomers. In certain embodiments, region C consists of 3 LNA monomers. In certain embodiments, region B consists of 7 nucleoside monomers. In certain embodiments, region B consists of 8 nucleoside monomers. In certain embodiments, region B consists of 9 nucleoside monomers. In certain embodiments, region B comprises 1-9 DNA monomers, such as 2, 3, 4, 5, 6, 7 or 8 DNA monomers. In certain embodiments, region B consists of DNA monomers. In certain embodiments, region B comprises at least one LNA monomer which is in the alpha-L configuration, such as 2, 3, 4, 5, 6, 7, 8 or 9 LNA monomers in the alpha-L-configuration. In certain embodiments, region B comprises at least one alpha-L-oxy LNA monomer. In certain embodiments, all of the LNA monomers in region B that are in the alpha-L-configuration are alpha-L-oxy LNA monomers. In certain embodiments, the number of monomers present in the A-B-C regions of the oligomers is selected from the group consisting of (nucleoside analog monomers—region B—nucleoside analog monomers): 1-8-1, 1-8-2, 2-8-1, 2-8-2, 3-8-3, 2-8-3, 3-8-2, 4-8-1, 4-8-2, 1-8-4, 2-8-4, or; 1-9-1, 1-9-2, 2-9-1, 2-9-2, 2-9-3, 3-9-2, 1-9-3, 3-9-1, 4-9-1, 1-9-4, or; 1-10-1, 1-10-2, 2-10-1, 2-10-2, 1-10-3, and 3-10-1. In certain embodiments, the number of monomers present in the A-B-C regions of the oligomers of the invention is selected from the group consisting of: 2-7-1, 1-7-2, 2-7-2, 3-7-3, 2-7-3, 3-7-2, 3-7-4, and 4-7-3. In certain embodiments, each of regions A and C consists of two LNA monomers, and region B consists of 8 or 9 nucleoside monomers, which in certain embodiments are DNA monomers.

In various embodiments, other gapmer designs include those where regions A and/or C consists of 3, 4, 5 or 6 nucleoside analogs, such as monomers containing a 2′-O-methoxyethyl-ribose sugar (2′MOE) or monomers containing a 2′-fluoro-deoxyribose sugar, and region B consists of 8, 9, 10, 11 or 12 nucleosides, such as DNA monomers, where regions A-B-C have 5-10-5 or 4-12-4 monomers.

In some embodiments, the gapmers contain sulfur-containing linkage groups as provided herein. In various embodiments, the gapmers contain phosphorothioate linkage groups, particularly in the gap region (B).

In certain embodiments, phosphorothioate linkages link together monomers in the flanking regions (A and C). In various embodiments, phosphorothioate linkages link regions A or C to region D, and link together monomers within region D.

In various embodiments, regions A, B and C comprise linkage groups other than phosphorothioate, such as phosphodiester linkages, particularly, for instance when the use of nucleoside analogs (e.g., LNA monomers) protects the linkage groups within regions A and C from endonuclease degradation.

In various embodiments, adjacent monomers of the oligomer are linked to each other by means of phosphorothioate groups.

It is recognized that the inclusion of phosphodiester linkages, such as one or two linkages, into an oligomer with a phosphorothioate backbone, particularly with phosphorothioate linkage groups between or adjacent to nucleoside analog monomers (typically in region A and/or C), can modify the bioavailability and/or bio-distribution of an oligomer—see WO 2008/053314, hereby incorporated by reference.

In some embodiments, such as the embodiments referred to above, where suitable and not specifically indicated, all remaining linkage groups are either phosphodiester or phosphorothioate, or a mixture thereof.

In some embodiments all the internucleoside linkage groups are phosphorothioate.

When referring to specific gapmer oligonucleotide sequences, such as those provided herein, it will be understood that, in various embodiments, when the linkages are phosphorothioate linkages, alternative linkages, such as those disclosed herein, may be used, for example phosphate (phosphodiester) linkages may be used, particularly for linkages between nucleoside analogs, such as LNA monomers.

Additional gapmer designs are disclosed in WO 2004/046160 and WO 2007/146511A2, which are hereby incorporated by reference. U.S. provisional application, 60/977,409, hereby incorporated by reference, refers to “shortmer” gapmer oligomers. In some embodiments, oligomers presented here may be such shortmer gapmers.

1.5.5. Sequences and Specificities of Oligomers

The oligomers that are used in the compositions and methods of the invention hybridize to nucleic acids that encode HER3 and/or HER2 and/or EGFR polypeptides.

The terms “nucleic acid” and “polynucleotide” are used interchangeably herein, and are defined as a molecule formed by covalent linkage of two or more monomers, as above-described. Including 2 or more monomers, “nucleic acids” may be of any length, and the term is generic to “oligomers”, which have the lengths described herein. The terms “nucleic acid” and “polynucleotide” include single-stranded, double-stranded, partially double-stranded, and circular molecules.

In various embodiments, the term “target nucleic acid”, as used herein, refers to the nucleic acid (such as DNA or RNA) encoding mammalian HER3 polypeptide (e.g., such as human HER3 mRNA having the sequence in SEQ ID NO 197, or mammalian mRNAs having GenBank Accession numbers NM_{—}001005915, NM_{—}001982 and alternatively-spliced forms NP_{—}001973.2 and NP_{—}001005915.1 (human); NM_{—}017218 (rat); NM_{—}010153 (mouse); NM_{—}001103105 (cow); or predicted mRNA sequences having GenBank Accession numbers XM_{—}001491896 (horse), XM_{—}001169469 and XM_{—}509131 (chimpanzee)).

In various embodiments, “target nucleic acid” also includes a nucleic acid encoding a mammalian HER2 polypeptide (e.g., such mammalian mRNAs having GenBank Accession numbers NM_{—}001005862 and NM_{—}004448 (human); NM_{—}017003 and NM_{—}017218 (rat); NM_{—}001003817 (mouse); NM_{—}001003217 (dog); and NM_{—}001048163 (cat)).

In various embodiments, “target nucleic acid” also includes a nucleic acid encoding a mammalian EGFR polypeptide (e.g., such as mammalian mRNAs having GenBank Accession numbers NM_{—}201284, NM_{—}201283, NM_{—}201282 and NM_{—}005228 (human); NM_{—}007912 and NM_{—}207655 (mouse); NM_{—}031507 (rat); and NM_{—}214007 (pig)).

It is recognized that the above-disclosed GenBank Accession numbers refer to cDNA sequences and not to mRNA sequences per se. The sequence of a mature mRNA can be derived directly from the corresponding cDNA sequence, with thymine bases (T) being replaced by uracil bases (U).

In various embodiments, “target nucleic acid” also includes HER3 (and optionally one or more of HER2 and EGFR) encoding nucleic acids or naturally occurring variants thereof, and RNA nucleic acids derived therefrom, such as pre-mRNA or mature mRNA. The oligomers according to the invention are typically capable of hybridizing to the target nucleic acid.

The term “naturally occurring variant thereof” refers to variants of the HER3 (or HER2 or EGFR) polypeptide or nucleic acid sequence which exist naturally within the defined taxonomic group, such as mammalian, such as mouse, monkey, and human. Typically, when referring to “naturally occurring variants” of a polynucleotide the term also may encompass any allelic variant of the HER3 (or HER2 or EGFR) encoding genomic DNA which is found at the Chromosome Chr 12: 54.76-54.78 Mb by chromosomal translocation or duplication, and the RNA, such as mRNA derived therefrom. When referenced to a specific polypeptide sequence, e.g., the term also includes naturally occurring forms of the protein which may therefore be processed, e.g. by co- or post-translational modifications, such as signal peptide cleavage, proteolytic cleavage, glycosylation, etc.

In certain embodiments, oligomers described herein bind to a region of the target nucleic acid (the “target region”) by either Watson-Crick base pairing, Hoogsteen hydrogen bonding, or reversed Hoogsteen hydrogen bonding, between the monomers of the oligomer and monomers of the target nucleic acid. Such binding is also referred to as “hybridization.” Unless otherwise indicated, binding is by Watson-Crick pairing of complementary bases (i.e., adenine with thymine (DNA) or uracil (RNA), and guanine with cytosine), and the oligomer binds to the target region because the sequence of the oligomer is identical to, or partially-identical to, the sequence of the reverse complement of the target region; for purposes herein, the oligomer is said to be “complementary” or “partially complementary” to the target region, and the percentage of “complementarily” of the oligomer sequence to that of the target region is the percentage “identity” to the reverse complement of the sequence of the target region.

Unless otherwise made clear by context, the “target region” herein will be the region of the target nucleic acid having the sequence that best aligns with the reverse complement of the sequence of the specified oligomer (or region thereof), using the alignment program and parameters described herein below.

In determining the degree of “complementarity” between oligomers for use in the compositions and methods of the invention (or regions thereof) and the target region of the nucleic acid which encodes mammalian HER3 (or HER2 or EGFR), such as those disclosed herein, the degree of “complementarity” (also, “homology”) is expressed as the percentage identity between the sequence of the oligomer (or region thereof) and the reverse complement of the sequence of the target region that best aligns therewith. The percentage is calculated by counting the number of aligned bases that are identical as between the 2 sequences, dividing by the total number of contiguous monomers in the oligomer (or region thereof), and multiplying by 100. In such a comparison, if gaps exist, it is preferable that such gaps are merely mismatches rather than areas where the number of monomers within the gap differs between the oligomer of the invention and the target region.

Amino acid and polynucleotide alignments, percentage sequence identity, and degree of complementarity may be determined for purposes of the invention using the ClustalW algorithm using standard settings: see http://www.ebi.ac.uk/emboss/align/index.html, Method: EMBOSS::water (local): Gap Open=10.0, Gap extend=0.5, using Blosum 62 (protein), or DNAfull for nucleotide/nucleobase sequences.

As will be understood, depending on context, “mismatch” refers to a nonidentity in sequence (as, for example, between the nucleobase sequence of an oligomer and the reverse complement of the target region to which it binds; as for example, between the base sequence of two aligned HER3 encoding nucleic acids), or to noncomplementarity in sequence (as, for example, between an oligomer and the target region to which binds).

Suitably, the oligomer (or conjugate, as further described, below) is capable of inhibiting (such as, by down-regulating) expression of the HER3 (and optionally of one or more of HER2 and EGFR) gene.

In various embodiments, the oligomers used in the compositions and methods of the invention effect inhibition of HER3 (and optionally of one or more of HER2 and EGFR) mRNA expression of at least 10% as compared to the expression level immediately prior to treatment, at least 20%, and more preferably at least 30%, 40%, 50%, 60%, 70%, 80%, 90% or 95% as compared to the expression level immediately prior to treatment. In various embodiments, the oligomers of the invention effect inhibition of HER3 (and optionally of one or more of HER2 and EGFR) protein expression of at least 10% as compared to the expression level immediately prior to treatment, at least 20%, more preferably at least 30%, 40%, 50%, 60%, 70%, 80%, 90% or 95% as compared to the expression level immediately prior to treatment. In some embodiments, such inhibition is seen when using 1 nM of the oligomer or conjugate of the invention. In various embodiments, such inhibition is seen when using 25 nM of the oligomer or conjugate.

In various embodiments, the inhibition of mRNA expression is less than 100% (i.e., less than complete inhibition of expression), such as less than 98%, inhibition, less than 95% inhibition, less than 90% inhibition, less than 80% inhibition, such as less than 70% inhibition. In various embodiments, the inhibition of protein expression is less than 100% (i.e., less than complete inhibition of expression), such as less than 98%, inhibition, less than 95% inhibition, less than 90% inhibition, less than 80% inhibition, such as less than 70% inhibition.

Alternatively, modulation of expression levels can be determined by measuring levels of mRNA, e.g. by northern blotting or quantitative RT-PCR. When measuring via mRNA levels, the level of inhibition when using an appropriate dosage, such as 1 and 25 nM, is, in various embodiments, typically to a level of 10-20% of the levels in the absence of the compound of the invention.

Modulation (i.e., inhibition or increase) of expression level may also be determined by measuring protein levels, e.g. by methods such as SDS-PAGE followed by western blotting using suitable antibodies raised against the target protein.

In some embodiments, the invention provides oligomers that inhibit (e.g., down-regulate) the expression of one or more alternatively-spliced isoforms of HER3 mRNA and/or proteins derived therefrom. In some embodiments, the invention provides oligomers that inhibit expression of one or more of the alternatively-spliced protein isoforms of HER3 (GenBank Accession nos. NP_{—}001973.2 and NP_{—}001005915.1) and/or expression of the nucleic acids that encode the HER3 protein isoforms (GenBank Accession nos. NM_{—}001982 and NM_{—}001005915.1). In some embodiments, the mRNA encoding HER3 isoform 1 is the target nucleic acid. In other embodiments, the mRNA encoding HER3 isoform 2 is the target nucleic acid. In certain embodiments, the nucleic acids encoding HER3 isoform 1 and HER3 isoform 2 are target nucleic acids, for example, an oligomer having the sequence of SEQ ID NO: 180.

In various embodiments, the invention provides oligomers, or a first region thereof, having a base sequence that is complementary to the sequence of a target region in a HER3 nucleic acid, which oligomers down-regulate HER3 mRNA and/or HER3 protein expression and down-regulate the expression of mRNA and/or protein of one or more other ErbB receptor tyrosine kinase family members, such as HER2 and/or EGFR. Oligomers, or a first region thereof, that effectively bind to the target regions of two different ErbB receptor family nucleic acids (e.g., HER2 and HER3 mRNA) and that down-regulate the mRNA and/or protein expression of both targets are termed “bispecific.” Oligomers, or a first region thereof, that bind to the target regions of three different ErbB receptor family members and are capable of effectively down-regulating all three genes are termed “trispecific”. In various embodiments, an oligomeric compound of the invention may be polyspecific, i.e. capable of binding to target regions of target nucleic acids of multiple members of the ErbB family of receptor tyrosine kinases and down-regulating their expression. As used herein, the terms “bispecific” and “trispecific” are understood not to be limiting in any way. For example, a “bispecific oligomer” may have some effect on a third target nucleic acid, while a “trispecific oligomer” may have a very weak and therefore insignificant effect on one of its three target nucleic acids.

In various embodiments, bispecific oligomers, or a first region thereof, are capable of binding to a target region in a HER3 nucleic acid and a target region in a HER2 target nucleic acid and effectively down-regulating the expression of HER3 and HER2 mRNA and/or protein. In certain embodiments, the bispecific oligomers do not down-regulate expression of HER3 mRNA and/or protein and HER2 mRNA and/or protein to the same extent. In other embodiments, the bispecific oligomers of the invention, or a first region thereof, are capable of binding to a target region in a HER3 target nucleic acid and a target region in an EGFR target nucleic acid and effectively down-regulating the expression of HER3 mRNA and/or protein and EGFR mRNA and/or protein. In various embodiments, the bispecific oligomers do not down-regulate expression of HER3 mRNA and/or protein and EGFR mRNA and/or protein to the same extent. In still other embodiments, trispecific oligomers, or a first region thereof, are capable of binding to a target region in a HER3 target nucleic acid, and to target regions in two other ErbB family of receptor tyrosine kinase target nucleic acids and effectively down-regulating the expression of HER3 mRNA and/or protein and mRNA and/or protein of the two other members of the ErbB family of receptor tyrosine kinases. In various embodiments, the trispecific oligomers, or a first region thereof, are capable of effectively down-regulating the expression of HER3 mRNA and/or protein, the expression of HER2 mRNA and/or protein, and the expression of EGFR mRNA and/or protein. In various embodiments, the trispecific oligomers do not down-regulate expression of HER3 mRNA and/or protein, HER2 mRNA and/or protein and EGFR mRNA and/or protein to the same extent.

An oligomer for use in the pharmaceutical compositions and methods of the invention typically binds to a target region of the human HER3 and/or the human HER2 and/or the human EGFR mRNA, and as such, comprises or consists of a region having a base sequence that is complementary or partially complementary to the base sequence of, e.g., SEQ ID NO 197, SEQ ID NO: 198 and/or SEQ ID NO: 199. In certain embodiments, the sequence of the oligomers for use in the pharmaceutical compositions and methods of the invention optionally comprise 1, 2, 3, 4 or more base mismatches when compared to the sequence of the best-aligned target region of SEQ ID NOs: 197, 198 or 199.

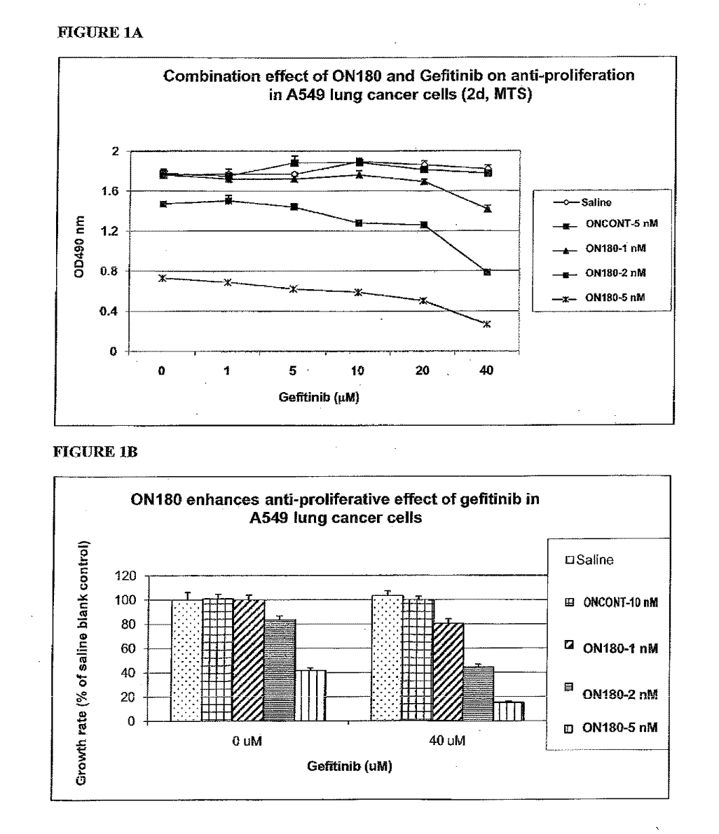

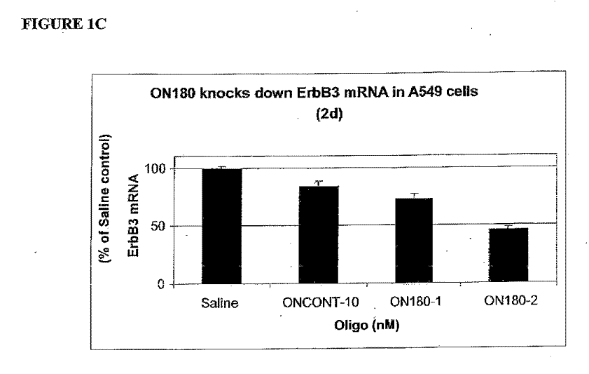

In some embodiments, the oligomers used in the pharmaceutical compositions and methods of the invention have sequences that are identical to a sequence selected from the group consisting of SEQ ID NOs: 200-227, 1-140 and 228-233 (see Table 1 herein below). In other embodiments, the oligomers used in the compositions and methods of the invention have sequences that differ in one, two, or three bases when compared to a sequence selected from the group consisting of SEQ ID NOs: 200-227, 1-140 and 228-233. In some embodiments, the oligomers comprise 10-16 contiguous monomers. Examples of the sequences of oligomers consisting of 16 contiguous monomers are SEQ ID NOs: 1, 16, 17, 18, 19, 34, 49, 50, 51, 52, 53, 54, 55, 56, 57, 58, 59, 74, 75, 76, 91, 92, 107, 122, 137, 138, 139, and 140. Shorter sequences can be derived therefrom, e.g., the sequence of the shorter oligomer may be identically present in a region of an oligomer selected from those having base sequences of SEQ ID NOs: 200-227, 1-140 and 228-233. In various embodiments, longer oligomers include a region having a sequence of at least 10 contiguous monomers that is identically present in SEQ ID NOs: 200-227, 1-140 and 228-233. Target regions of human HER3 mRNA which are complementary to the oligomers having sequences of SEQ ID NOs: 1, 16, 17, 18, 19, 34, 49, 50, 51, 52, 53, 54, 55, 56, 57, 58, 59, 74, 75, 76, 91, 92, 107, 122, 137, 138, 139, and 140 are shown in FIG. 1 (bold and underlined, with the corresponding oligomer SEQ ID NOs indicated above).

In various embodiments, the oligomers have the base sequences shown in SEQ ID NOs: 141-168. In certain embodiments, the oligomers are LNA oligomers, for example, those having the sequences of SEQ ID NOS: 169-196 and 234, in particular those having the base sequences of SEQ ID NOs: 169, 170, 173, 174, 180, 181, 183, 185, 187, 188, 189, 190, 191, 192 and 194. In various embodiments, the oligomers are LNA oligomers such as those having base sequences of SEQ ID NOs: 169, 170, 172, 174, 175, 176 and 179. In some embodiments, the oligomers or a region thereof consist of or comprise a base sequence as shown in SEQ ID NOs: 169, 180 or 234. In some embodiments, conjugates of the invention include an oligomer having a base sequence as shown in SEQ ID NOs: 169, 180 or 234.

In certain embodiments, the oligomer used in the compositions and methods of the invention may, suitably, comprise a region having a particular sequence, such as a sequence selected from SEQ ID NOs: 200-227, that is identically present in a shorter oligomer, which may also be used in the compositions and methods of the invention. In various embodiments, the region comprises 10-16 monomers. For example, the oligomers having the base sequences of SEQ ID NOs: 200-227 each comprise a region wherein the sequence of the region is identically present in shorter oligomers having sequences of SEQ ID NOs: 1, 16, 17, 18, 19, 34, 49, 50, 51, 52, 53, 54, 55, 56, 57, 58, 59, 74, 75, 76, 91, 92, 107, 122, 137, 138, 139, and 140, respectively. In some embodiments, oligomers that have fewer than 16 monomers, such as 10, 11, 12, 13, 14, or 15 monomers, have a region of at least 8, at least 9, at least 10, at least 11, at least 12, at least 13, at least 14 or 15, contiguous monomers of which the sequence is identically present in oligomers having sequences of SEQ ID NOS: 1, 16, 17, 18, 19, 34, 49, 50, 51, 52, 53, 54, 55, 56, 57, 58, 59, 74, 75, 76, 91, 92, 107, 122, 137, 138, 139, or 140. Hence, in various embodiments, the sequences of shorter oligomers are derived from the sequences of longer oligomers. In some embodiments, the sequences of oligomers having SEQ ID NOs disclosed herein, or the sequences of at least 10 contiguous monomers thereof, are identically present in longer oligomers. Typically an oligomer for use in the pharmaceutical compositions and methods of the invention comprises a first region having a sequence that is identically present in SEQ ID NOs: 1, 16, 17, 18, 19, 34, 49, 50, 51, 52, 53, 54, 55, 56, 57, 58, 59, 74, 75, 76, 91, 92, 107, 122, 137, 138, 139, or 140, and if the oligomer is longer than the first region that is identically present in SEQ ID NOs: 1, 16, 17, 18, 19, 34, 49, 50, 51, 52, 53, 54, 55, 56, 57, 58, 59, 74, 75, 76, 91, 92, 107, 122, 137, 138, 139, or 140, the flanking regions of the oligomer have sequences that are complementary to the sequences flanking the target region of the target nucleic acid. Two such oligomers are SEQ ID NO: 1 and SEQ ID NO: 54.

In various embodiments, the oligomer comprises or consists of a sequence of monomers which is fully complementary (perfectly complementary) to a target region of a target nucleic acid which encodes a mammalian HER3.

However, in some embodiments, the sequence of the oligomer includes 1, 2, 3, or 4 (or more) mismatches as compared to the best-aligned target region of a HER3 target nucleic acid, and still sufficiently binds to the target region to effect inhibition of HER3 mRNA or protein expression. The destabilizing effect of mismatches on the Watson-Crick hydrogen-bonded duplex may, for example, be compensated by increased length of the oligomer and/or an increased number of nucleoside analogs, such as LNA monomers, present within the oligomer.

In various embodiments, the oligomer base sequence comprises no more than 3, such as no more than 2 mismatches compared to the base sequence of the best-aligned target region of, for example, a target nucleic acid which encodes a mammalian HER3.

The base sequences of the oligomers for use in the compositions and methods of the invention or of a region thereof are in various embodiments at least 80% identical to a sequence selected from the group consisting of SEQ ID NOS: 200-227, 1-140 and 228-233, such as at least 85%, at least 90%, at least 91%, at least 92%, at least 93%, at least 94%, at least 95%, at least 96%, at least 97%, at least 98%, at least 99%, even 100% identical.

The base sequences of the oligomers or of a first region thereof are in various embodiments at least 80% complementary to a sequence of a target region present in SEQ ID NOs: 197, 198 and/or 199 such as at least 85%, at least 90%, at least 91%, at least 92%, at least 93%, at least 94%, at least 95%, at least 96%, at least 97%, at least 98%, at least 99%, even 100% complementary.

In various embodiments, the sequence of the oligomer (or a first region thereof) is selected from the group consisting of SEQ ID NOs: 200-227, 1-140 and 228 233, or is selected from the group consisting of at least 10 contiguous monomers of SEQ ID NOs: 200-227, 1-140 and 228-233. In other embodiments, the sequence of the oligomer used in the pharmaceutical compositions and methods of the invention or a first region thereof optionally comprises 1, 2 or 3 base moieties that differ from those in oligomers having sequences of SEQ ID NOs: 200-227, 1-140 and 228-233, or the sequences of at least 10 contiguous monomers thereof, when optimally aligned with the selected sequence or region thereof.

In certain embodiments, the monomer region consists of 11, 12, 13, 14, 15, 16, 17, 18, 19, 20, 21, 22, 23, 24, 25, 26, 27, 28, or 29 contiguous monomers, such as between 10-15, 12-25, 12-22, such as between 12-18 monomers. Suitably, in various embodiments, the region is of the same length as the oligomer of the invention.

In some embodiments, the oligomer comprises additional monomers at the 5′ or 3′ ends, such as, independently, 1, 2, 3, 4 or 5 additional monomers 5′ end and/or 3′ end of the oligomer, which are non-complementary to the sequence of the target region. In various embodiments, the oligomer of the invention comprises a region that is complementary to the target, which is flanked 5′ and/or 3′ by additional monomers. In various embodiments, the 3′ end of the region is flanked by 1, 2 or 3 DNA or RNA monomers. 3′ DNA monomers are frequently used during solid state synthesis of oligomers. In various embodiments, which may be the same or different, the 5′ end of the oligomer is flanked by 1, 2 or 3 DNA or RNA monomers. In certain embodiments, the additional 5′ or 3′ monomers are nucleosides, such as DNA or RNA monomers. In various embodiments, the 5′ or 3′ monomers may represent region D as referred to in the context of gapmer oligomers herein.

TABLE 1
Oligomer Sequences

| | | | Target | | |
|---|---|---|---|---|---|
| | | Length | site | Compl | Compl |
| SEQ ID NO | Sequence (5′-3′) | (bases) | HER3 | EGFR | HER2 |
| SEQ ID NO: 1 | GCTCCAGACATCACTC | 16 | 2866- | 100% | 87.5% |
| | | | 2881 | | |
| SEQ ID NO: 2 | GCTCCAGACATCACT | 15 | | | |
| SEQ ID NO: 3 | CTCCAGACATCACTC | 15 | | | |
| SEQ ID NO: 4 | GCTCCAGACATCAC | 14 | | | |
| SEQ ID NO: 5 | CTCCAGACATCACT | 14 | | | |
| SEQ ID NO: 6 | TCCAGACATCACTC | 14 | | | |
| SEQ ID NO: 7 | GCTCCAGACATCA | 13 | | | |
| SEQ ID NO: 8 | CTCCAGACATCAC | 13 | | | |
| SEQ ID NO: 9 | TCCAGACATCACT | 13 | | | |
| SEQ ID NO: 10 | CCAGACATCACTC | 13 | | | |
| SEQ ID NO: 11 | GCTCCAGACATC | 12 | | | |
| SEQ ID NO: 12 | CTCCAGACATCA | 12 | | | |
| SEQ ID NO: 13 | TCCAGACATCAC | 12 | | | |
| SEQ ID NO: 14 | CCAGACATCACT | 12 | | | |
| SEQ ID NO: 15 | CAGACATCACTC | 12 | | | |
| SEQ ID NO: 16 | CTCCAGACATCACTCT | 16 | 2865- | 100% | 93.8% |
| | | | 2880 | | |
| SEQ ID NO: 17 | CAGACATCACTCTGGT | 16 | 2862- | 100% | 93.8% |
| | | | 2877 | | |
| SEQ ID NO: 18 | AGACATCACTCTGGTG | 16 | 2861- | 100% | 93.8% |
| | | | 2876 | | |
| SEQ ID NO: 19 | ATAGCTCCAGACATCA | 16 | 2869- | 93.8% | 87.5% |
| | | | 2884 | | |
| SEQ ID NO: 20 | ATAGCTCCAGACATC | 15 | | | |
| SEQ ID NO: 21 | TAGCTCCAGACATCA | 15 | | | |
| SEQ ID NO: 22 | ATAGCTCCAGACAT | 14 | | | |
| SEQ ID NO: 23 | TAGCTCCAGACATC | 14 | | | |
| SEQ ID NO: 24 | AGCTCCAGACATCA | 14 | | | |
| SEQ ID NO: 25 | ATAGCTCCAGACA | 13 | | | |
| SEQ ID NO: 26 | TAGCTCCAGACAT | 13 | | | |
| SEQ ID NO: 27 | AGCTCCAGACATC | 13 | | | |
| SEQ ID NO: 28 | GCTCCAGACATCA | 13 | | | |
| SEQ ID NO: 29 | ATAGCTCCAGAC | 12 | | | |
| SEQ ID NO: 30 | TAGCTCCAGACA | 12 | | | |
| SEQ ID NO: 31 | AGCTCCAGACAT | 12 | | | |
| SEQ ID NO: 32 | GCTCCAGACATC | 12 | | | |
| SEQ ID NO: 33 | CTCCAGACATCA | 12 | | | |
| SEQ ID NO: 34 | TCACACCATAGCTCCA | 16 | 2876- | 87.5% | 93.8% |
| | | | 2891 | | |
| SEQ ID NO: 35 | TCACACCATAGCTCC | 15 | | | |
| SEQ ID NO: 36 | CACACCATAGCTCCA | 15 | | | |
| SEQ ID NO: 37 | TCACACCATAGCTC | 14 | | | |
| SEQ ID NO: 38 | CACACCATAGCTCC | 14 | | | |
| SEQ ID NO: 39 | ACACCATAGCTCCA | 14 | | | |
| SEQ ID NO: 40 | TCACACCATAGCT | 13 | | | |
| SEQ ID NO: 41 | CACACCATAGCTC | 13 | | | |
| SEQ ID NO: 42 | ACACCATAGCTCC | 13 | | | |
| SEQ ID NO: 43 | CACCATAGCTCCA | 13 | | | |
| SEQ ID NO: 44 | TCACACCATAGC | 12 | | | |
| SEQ ID NO: 45 | CACACCATAGCT | 12 | | | |
| SEQ ID NO: 46 | ACACCATAGCTC | 12 | | | |
| SEQ ID NO: 47 | CACCATAGCTCC | 12 | | | |
| SEQ ID NO: 48 | ACCATAGCTCCA | 12 | | | |
| SEQ ID NO: 49 | CATCCAACACTTGACC | 16 | 3025- | 93.8% | 93.8% |
| | | | 3040 | | |
| SEQ ID NO: 50 | ATCCAACACTTGACCA | 16 | 3024- | 93.8% | 93.8% |
| | | | 3039 | | |
| SEQ ID NO: 51 | CAATCATCCAACACTT | 16 | 3029- | 87.5% | 93.8% |
| | | | 3044 | | |
| SEQ ID NO: 52 | TCAATCATCCAACACT | 16 | 3030- | 87.5% | 93.8% |
| | | | 3045 | | |
| SEQ ID NO: 53 | CATGTAGACATCAATT | 16 | 3004- | 87.5% | 93.8% |
| | | | 3019 | | |
| SEQ ID NO: 54 | TAGCCTGTCACTTCTC | 16 | 435- | 68.8% | 75% |
| | | | 450 | | |
| SEQ ID NO: 55 | AGATGGCAAACTTCCC | 16 | 530- | 68.8% | 68.8% |
| | | | 545 | | |
| SEQ ID NO: 56 | CAAGGCTCACACATCT | 16 | 1146 | 75% | 68.8% |
| | | | 1161 | | |
| SEQ ID NO: 57 | AAGTCCAGGTTGCCCA | 16 | 1266 | 75% | 75% |
| | | | 1281 | | |
| SEQ ID NO: 58 | CATTCAAGTTCTTCAT | 16 | 1490 | 75% | 68.8% |
| | | | 1505 | | |
| SEQ ID NO: 59 | CACTAATTTCCTTCAG | 16 | 1529 | 81.3% | 68.8% |
| | | | 1544 | | |
| SEQ ID NO: 60 | CACTAATTTCCTTCA | 15 | | | |
| SEQ ID NO: 61 | ACTAATTTCCTTCAG | 15 | | | |
| SEQ ID NO: 62 | CACTAATTTCCTTC | 14 | | | |
| SEQ ID NO: 63 | ACTAATTTCCTTCA | 14 | | | |
| SEQ ID NO: 64 | CTAATTTCCTTCAG | 14 | | | |
| SEQ ID NO: 65 | CACTAATTTCCTT | 13 | | | |
| SEQ ID NO: 66 | ACTAATTTCCTTC | 13 | | | |
| SEQ ID NO: 67 | CTAATTTCCTTCA | 13 | | | |
| SEQ ID NO: 68 | TAATTTCCTTCAG | 13 | | | |
| SEQ ID NO: 69 | CACTAATTTCCT | 12 | | | |
| SEQ ID NO: 70 | ACTAATTTCCTT | 12 | | | |
| SEQ ID NO: 71 | CTAATTTCCTTC | 12 | | | |
| SEQ ID NO: 72 | TAATTTCCTTCA | 12 | | | |
| SEQ ID NO: 73 | AATTTCCTTCAG | 12 | | | |
| SEQ ID NO: 74 | GCCCAGCACTAATTTC | 16 | 1535- | 75% | 68.8% |
| | | | 1550 | | |
| SEQ ID NO: 75 | CTTTGCCCTCTGCCAC | 16 | 1673- | 75% | 75% |
| | | | 1688 | | |
| SEQ ID NO: 76 | CACACACTTTGCCCTC | 16 | 1679- | 68.8% | 75% |
| | | | 1694 | | |
| SEQ ID NO: 77 | CACACACTTTGCCCT | 15 | | | |
| SEQ ID NO: 78 | ACACACTTTGCCCTC | 15 | | | |
| SEQ ID NO: 79 | CACACACTTTGCCC | 14 | | | |
| SEQ ID NO: 80 | ACACACTTTGCCCT | 14 | | | |
| SEQ ID NO: 81 | CACACTTTGCCCTC | 14 | | | |
| SEQ ID NO: 82 | CACACACTTTGCC | 13 | | | |
| SEQ ID NO: 83 | ACACACTTTGCCC | 13 | | | |
| SEQ ID NO: 84 | CACACTTTGCCCT | 13 | | | |
| SEQ ID NO: 85 | ACACTTTGCCCTC | 13 | | | |
| SEQ ID NO: 86 | CACACACTTTGC | 12 | | | |
| SEQ ID NO: 87 | ACACACTTTGCC | 12 | | | |
| SEQ ID NO: 88 | CACACTTTGCCC | 12 | | | |
| SEQ ID NO: 89 | ACACTTTGCCCT | 12 | | | |
| SEQ ID NO: 90 | CACTTTGCCCTC | 12 | | | |
| SEQ ID NO: 91 | CAGTTCCAAAGACACC | 16 | 2345- | 75% | 68.8% |
| | | | 2360 | | |
| SEQ ID NO: 92 | TGGCAATTTGTACTCC | 16 | 2636- | 75% | 68.8% |
| | | | 2651 | | |
| SEQ ID NO: 93 | TGGCAATTTGTACTC | 15 | | | |
| SEQ ID NO: 94 | GGCAATTTGTACTCC | 15 | | | |
| SEQ ID NO: 95 | TGGCAATTTGTACT | 14 | | | |
| SEQ ID NO: 96 | GGCAATTTGTACTC | 14 | | | |
| SEQ ID NO: 97 | GCAATTTGTACTCC | 14 | | | |
| SEQ ID NO: 98 | TGGCAATTTGTAC | 13 | | | |
| SEQ ID NO: 99 | GGCAATTTGTACT | 13 | | | |
| SEQ ID NO: 100 | GCAATTTGTACTC | 13 | | | |
| SEQ ID NO: 101 | CAATTTGTACTCC | 13 | | | |
| SEQ ID NO: 102 | TGGCAATTTGTA | 12 | | | |
| SEQ ID NO: 103 | GGCAATTTGTAC | 12 | | | |
| SEQ ID NO: 104 | GCAATTTGTACT | 12 | | | |
| SEQ ID NO: 105 | CAATTTGTACTC | 12 | | | |
| SEQ ID NO: 106 | AATTTGTACTCC | 12 | | | |
| SEQ ID NO: 107 | GTGTGTGTATTTCCCA | 16 | 2848- | 75% | 68.8% |
| | | | 2863 | | |
| SEQ ID NO: 108 | GTGTGTGTATTTCCC | 15 | | | |
| SEQ ID NO: 109 | TGTGTGTATTTCCCA | 15 | | | |
| SEQ ID NO: 110 | GTGTGTGTATTTCC | 14 | | | |
| SEQ ID NO: 111 | TGTGTGTATTTCCC | 14 | | | |
| SEQ ID NO: 112 | GTGTGTATTTCCCA | 14 | | | |
| SEQ ID NO: 113 | GTGTGTGTATTTC | 13 | | | |
| SEQ ID NO: 114 | TGTGTGTATTTCC | 13 | | | |
| SEQ ID NO: 115 | GTGTGTATTTCCC | 13 | | | |
| SEQ ID NO: 116 | TGTGTATTTCCCA | 13 | | | |
| SEQ ID NO: 117 | GTGTGTGTATTT | 12 | | | |
| SEQ ID NO: 118 | TGTGTGTATTTC | 12 | | | |
| SEQ ID NO: 119 | GTGTGTATTTCC | 12 | | | |
| SEQ ID NO: 120 | TGTGTATTTCCC | 12 | | | |
| SEQ ID NO: 121 | GTGTATTTCCCA | 12 | | | |
| SEQ ID NO: 122 | CCCTCTGATGACTCTG | 16 | 3474- | 68.8% | 68.8% |
| | | | 3489 | | |
| SEQ ID NO: 123 | CCCTCTGATGACTCT | 15 | | | |
| SEQ ID NO: 124 | CCTCTGATGACTCTG | 15 | | | |
| SEQ ID NO: 125 | CCCTCTGATGACTC | 14 | | | |
| SEQ ID NO: 126 | CCTCTGATGACTCT | 14 | | | |
| SEQ ID NO: 127 | CTCTGATGACTCTG | 14 | | | |
| SEQ ID NO: 128 | CCCTCTGATGACT | 13 | | | |
| SEQ ID NO: 129 | CCTCTGATGACTC | 13 | | | |
| SEQ ID NO: 130 | CTCTGATGACTCT | 13 | | | |
| SEQ ID NO: 131 | TCTGATGACTCTG | 13 | | | |
| SEQ ID NO: 132 | CCCTCTGATGAC | 12 | | | |
| SEQ ID NO: 133 | CCTCTGATGACT | 12 | | | |
| SEQ ID NO: 134 | CTCTGATGACTC | 12 | | | |
| SEQ ID NO: 135 | TCTGATGACTCT | 12 | | | |
| SEQ ID NO: 136 | CTGATGACTCTG | 12 | | | |
| SEQ ID NO: 137 | CATACTCCTCATCTTC | 16 | 3770- | 81.3% | 81.3% |
| | | | 3785 | | |
| SEQ ID NO: 138 | CCACCACAAAGTTATG | 16 | 1067- | 81.3% | 68.8% |
| | | | 1082 | | |
| SEQ ID NO: 139 | CATCACTCTGGTGTGT | 16 | 2858- | 93.8% | 93.8% |
| | | | 2873 | | |
| SEQ ID NO: 140 | GACATCACTCTGGTGT | 16 | 2860- | 93.8% | 87.5% |
| | | | 2875 | | |
| SEQ ID NO: 141 | [symbol] | 16 | | | |
| SEQ ID NO: 142 | [symbol] | 16 | | | |
| SEQ ID NO: 143 | [symbol] | 16 | | | |
| SEQ ID NO: 144 | [symbol] | 16 | | | |
| SEQ ID NO: 145 | [symbol] | 16 | | | |
| SEQ ID NO: 146 | [symbol] | 16 | | | |
| SEQ ID NO: 147 | [symbol] | 16 | | | |
| SEQ ID NO: 148 | [symbol] | 16 | | | |
| SEQ ID NO: 149 | [symbol] | 16 | | | |
| SEQ ID NO: 150 | [symbol] | 16 | | | |
| SEQ ID NO: 151 | [symbol] | 16 | | | |
| SEQ ID NO: 152 | [symbol] | 16 | | | |
| SEQ ID NO: 153 | [symbol] | 16 | | | |
| SEQ ID NO: 154 | [symbol] | 16 | | | |
| SEQ ID NO: 155 | [symbol] | 16 | | | |
| SEQ ID NO: 156 | [symbol] | 16 | | | |
| SEQ ID NO: 157 | [symbol] | 16 | | | |
| SEQ ID NO: 158 | [symbol] | 16 | | | |
| SEQ ID NO: 159 | [symbol] | 16 | | | |
| SEQ ID NO: 160 | [symbol] | 16 | | | |
| SEQ ID NO: 161 | [symbol] | 16 | | | |
| SEQ ID NO: 162 | [symbol] | 16 | | | |
| SEQ ID NO: 163 | [symbol] | 16 | | | |
| SEQ ID NO: 164 | [symbol] | 16 | | | |
| SEQ ID NO: 165 | [symbol] | 16 | | | |
| SEQ ID NO: 166 | [symbol] | 16 | | | |
| SEQ ID NO: 167 | [symbol] | 16 | | | |
| SEQ ID NO: 168 | [symbol] | 16 | | | |
| SEQ ID NO: 169 | [symbol] | 16 | | | |
| SEQ ID NO: 170 | [symbol] | 16 | | | |
| SEQ ID NO: 171 | [symbol] | 16 | | | |
| SEQ ID NO: 172 | [symbol] | 16 | | | |
| SEQ ID NO: 173 | [symbol] | 16 | | | |
| SEQ ID NO: 174 | [symbol] | 16 | | | |
| SEQ ID NO: 175 | [symbol] | 16 | | | |
| SEQ ID NO: 176 | [symbol] | 16 | | | |
| SEQ ID NO: 177 | [symbol] | 16 | | | |
| SEQ ID NO: 178 | [symbol] | 16 | | | |
| SEQ ID NO: 179 | [symbol] | 16 | | | |
| SEQ ID NO: 180 | [symbol] | 16 | | | |
| SEQ ID NO: 181 | [symbol] | 16 | | | |
| SEQ ID NO: 182 | [symbol] | 16 | | | |
| SEQ ID NO: 183 | [symbol] | 16 | | | |
| SEQ ID NO: 184 | [symbol] | 16 | | | |
| SEQ ID NO: 185 | [symbol] | 16 | | | |
| SEQ ID NO: 186 | [symbol] | 16 | | | |
| SEQ ID NO: 187 | [symbol] | 16 | | | |
| SEQ ID NO: 188 | [symbol] | 16 | | | |
| SEQ ID NO: 189 | [symbol] | 16 | | | |
| SEQ ID NO: 190 | [symbol] | 16 | | | |
| SEQ ID NO: 191 | [symbol] | 16 | | | |
| SEQ ID NO: 192 | [symbol] | 16 | | | |
| SEQ ID NO: 193 | [symbol] | 16 | | | |
| SEQ ID NO: 194 | [symbol] | 16 | | | |
| SEQ ID NO: 195 | [symbol] | 16 | | | |
| SEQ ID NO: 196 | [symbol] | 16 | | | |
| SEQ ID NO: 200 | CATAGCTCCAGACATCACTCTGGT | 24 | | | |
| SEQ ID NO: 201 | ATAGCTCCAGACATCACTCTGGTG | 24 | | | |
| SEQ ID NO: 202 | GCTCCAGACATCACTCTGGTGTGT | 24 | | | |
| SEQ ID NO: 203 | CTCCAGACATCACTCTGGTGTGTG | 24 | | | |
| SEQ ID NO: 204 | CACCATAGCTCCAGACATCACTCT | 24 | | | |
| SEQ ID NO: 205 | ACTGTCACACCATAGCTCCAGACA | 24 | | | |
| SEQ ID NO: 206 | CAATCATCCAACACTTGACCATCA | 24 | | | |
| SEQ ID NO: 207 | AATCATCCAACACTTGACCATCAC | 24 | | | |
| SEQ ID NO: 208 | TCATCAATCATCCAACACTTGACC | 24 | | | |
| SEQ ID NO: 209 | CTCATCAATCATCCAACACTTGAC | 24 | | | |
| SEQ ID NO: 210 | TCACCATGTAGACATCAATTGTGC | 24 | | | |
| SEQ ID NO: 211 | GACATAGCCTGTCACTTCTCGAAT | 24 | | | |
| SEQ ID NO: 212 | ACGAAGATGGCAAACTTCCCATCG | 24 | | | |
| SEQ ID NO: 213 | CCCACAAGGCTCACACATCTTGAG | 24 | | | |
| SEQ ID NO: 214 | CAGAAAGTCCAGGITGCCCAGGAT | 24 | | | |
| SEQ ID NO: 215 | GTGACATTCAAGITCTTCATGATC | 24 | | | |
| SEQ ID NO: 216 | CCAGCACTAATTTCCTTCAGGGAT | 24 | | | |
| SEQ ID NO: 217 | ATACGCCCAGCACTAATTTCCTTC | 24 | | | |
| SEQ ID NO: 218 | CACACTTTGCCCTCTGCCACGCAG | 24 | | | |
| SEQ ID NO: 219 | GGGTCACACACTTTGCCCTCTGCC | 24 | | | |
| SEQ ID NO: 220 | TGCACAGTTCCAAAGACACCCGAG | 24 | | | |
| SEQ ID NO: 221 | CCCTTGGCAATTTGTACTCCCCAG | 24 | | | |
| SEQ ID NO: 222 | TCTGGTGTGTGTATTTCCCAAAGT | 24 | | | |
| SEQ ID NO: 223 | ATGCCCCTCTGATGACTCTGATGC | 24 | | | |
| SEQ ID NO: 224 | TATTCATACTCCTCATCTTCATCT | 24 | | | |
| SEQ ID NO: 225 | TGATCCACCACAAAGTTATGGGGA | 24 | | | |
| SEQ ID NO: 226 | CAGACATCACTCTGGTGTGTGTAT | 24 | | | |
| SEQ ID NO: 227 | TCCAGACATCACTCTGGTGTGTGT | 24 | | | |
| SEQ ID NO: 228 | TAGCCTGTCACTTCT | 15 | | | |
| SEQ ID NO: 229 | AGCCTGTCACTTCTC | 15 | | | |
| SEQ ID NO: 230 | TAGCCTGTCACTTC | 14 | | | |
| SEQ ID NO: 231 | AGCCTGTCACTTCT | 14 | | | |
| SEQ ID NO: 232 | TAGCCTGTCACTT | 13 | | | |
| SEQ ID NO: 233 | TAGCCTGTCACT | 12 | | | |
| SEQ ID NO: 234 | [symbol] | 13 | | | |

For gapmer sequences (SEQ ID NOs: 141-196 and 234), uppercase letters in boldface type indicate that the nucleoside contains an LNA sugar and lowercase letters indicate 2′-deoxynucleosides. The subscript “s” indicates a phosphorothioate linkage between adjacent nucleosides. All cytosine bases in LNA monomers are 5-methylcytosines. For oligonucleotides having 24 nucleosides (SEQ ID NOs: 211-227), bold and underlined letters, as shown in Table 1, indicate a base sequence of a shorter oligomeric compound that has been incorporated into the longer oligonucleotides.

1.5.6. Conjugates

In the context of this disclosure, the term “conjugate” indicates a compound formed by the covalent attachment (“conjugation”) of an oligomer, as described herein, to one or more moieties that are not themselves nucleic acids or monomers (“conjugated moiety”). Examples of such conjugated moieties include macromolecular compounds such as proteins, fatty acid chains, sugar residues, glycoproteins, polymers, or combinations thereof. Typically, proteins may be antibodies for a target protein. Typical polymers may be polyethylene glycol. WO 2007/031091 provides suitable moieties and conjugates, which are hereby incorporated by reference.

Accordingly, in some embodiments, the compositions and methods of the invention utilize a conjugate comprising an oligomer as herein described, and at least one conjugated moiety that is not a nucleic acid or monomer, covalently attached to the oligomer. Therefore, in certain embodiments, where an oligomer consists of contiguous monomers having a specified sequence of bases, as herein disclosed, the conjugate may also comprise at least one conjugated moiety that is covalently attached to the oligomer.

In various embodiments, conjugates may enhance the activity, cellular distribution or cellular uptake of an oligomer. Such moieties include, but are not limited to, antibodies, polypeptides, lipid moieties such as a cholesterol moiety, cholic acid, a thioether, e.g. Hexyl-s-tritylthiol, a thiocholesterol, an aliphatic chain, e.g., dodecandiol or undecyl residues, a phospholipids, e.g., di-hexadecyl-rac-glycerol or triethylammonium 1,2-di-o-hexadecyl-rac-glycero-3-h-phosphonate, a polyamine or a polyethylene glycol chain, an adamantane acetic acid, a palmityl moiety, an octadecylamine or hexylamino-carbonyl-oxycholesterol moiety.

In certain embodiments, the oligomer is conjugated to a moiety that increases the cellular uptake of oligomeric compounds.

In certain embodiments, the oligomers are conjugated to active drug substances, for example, aspirin, ibuprofen, a sulfa drug, an antidiabetic, an antibacterial or an antibiotic.

In certain embodiments, the conjugated moiety is a sterol, such as cholesterol.

In various embodiments, the conjugated moiety comprises or consists of a positively charged polymer, such as a positively charged peptide of, for example 1-50, such as 2-20 such as 3-10 amino acid residues in length, and/or polyalkylene oxide such as polyethylene glycol (PEG) or polypropylene glycol—see WO 2008/034123, hereby incorporated by reference. Suitably, the positively charged polymer, such as a polyalkylene oxide may be attached to the oligomer via a linker such as the releasable linker described in WO 2008/034123.

1.5.6.1.1. Activated Oligomers

The term “activated oligomer,” as used herein, refers to an oligomer, such as the oligomers described above, that is covalently linked (i.e., functionalized) to at least one functional moiety that permits covalent linkage of the oligomer to one or more conjugated moieties, i.e., moieties that are not themselves nucleic acids or monomers, to form the conjugates herein described. Typically, a functional moiety will comprise a chemical group that is capable of covalently bonding to the oligomer via, e.g., a 3′-hydroxyl group or the exocyclic NH₂ group of the adenine base, a spacer that in some embodiments is hydrophilic and a terminal group that is capable of binding to a conjugated moiety (e.g., an amino, sulfhydryl or hydroxyl group). In some embodiments, this terminal group is not protected, e.g., is an NH₂ group. In other embodiments, the terminal group is protected, for example, by any suitable protecting group such as those described in “Protective Groups in Organic Synthesis” by Theodora W Greene and Peter G M Wuts, 3rd edition (John Wiley & Sons, 1999). Examples of suitable hydroxyl protecting groups include esters such as acetate ester, aralkyl groups such as benzyl, diphenylmethyl, or triphenylmethyl, and tetrahydropyranyl. Examples of suitable amino protecting groups include benzyl, alpha-methylbenzyl, diphenylmethyl, triphenylmethyl, benzyloxycarbonyl, tert-butoxycarbonyl, and acyl groups such as trichloroacetyl or trifluoroacetyl.

In some embodiments, the functional moiety is self-cleaving. In other embodiments, the functional moiety is biodegradable. See e.g., U.S. Pat. No. 7,087,229, which is incorporated by reference herein in its entirety.

In some embodiments, the oligomers for use in the compositions and methods of the invention are functionalized at the 5′ end in order to allow covalent attachment of the conjugated moiety to the 5′ end of the oligomer. In other embodiments, the oligomers can be functionalized at the 3′ end. In still other embodiments, oligomers can be functionalized along the backbone or on the heterocyclic base moiety. In yet other embodiments, oligomers can be functionalized at more than one position independently selected from the 5′ end, the 3′ end, the backbone and the base.

In some embodiments, activated oligomers are synthesized by incorporating during the synthesis one or more monomers that is covalently attached to a functional moiety. In other embodiments, activated oligomers of the invention are synthesized with monomers that have not been functionalized, and the oligomer is functionalized upon completion of synthesis.

In some embodiments, the oligomers are functionalized with a hindered ester containing an aminoalkyl linker, wherein the alkyl portion has the formula (CH₂)_{w}, wherein w is an integer ranging from 1 to 10, such as about 6, wherein the alkyl portion of the alkylamino group can be straight chain or branched chain, and wherein the functional group is attached to the oligomer via an ester group (—O—C(O)—(CH₂)_{w}NH).

In other embodiments, the oligomers are functionalized with a hindered ester containing a (CH₂)_{w}-sulfhydryl (SH) linker, wherein w is an integer ranging from 1 to 10, such as about 6, wherein the alkyl portion of the alkylamino group can be straight chain or branched chain, and wherein the functional group attached to the oligomer via an ester group (—O—C(O)—(CH₂)_{w}SH). In some embodiments, sulfhydryl-activated oligonucleotides are conjugated with polymer moieties such as polyethylene glycol or peptides (via formation of a disulfide bond).

Activated oligomers covalently linked to at least one functional moiety can be synthesized by any method known in the art, and in particular by methods disclosed in U.S. Pat. No. 7,595,304, WO 2008/034122 and WO 2008/034119, each of which is incorporated herein by reference in its entirety, and in Zhao et al. (2007) J. Controlled Release 119:143-152; and Zhao et al. (2005) Bioconjugate Chem. 16:758-766.

In still other embodiments, the oligomers for use in the pharmaceutical compositions and methods of the invention are functionalized by introducing sulfhydryl, amino or hydroxyl groups into the oligomer by means of a functionalizing reagent substantially as described in U.S. Pat. Nos. 4,962,029 and 4,914,210, i.e., a substantially linear reagent having a phosphoramidite at one end linked through a hydrophilic spacer chain to the opposing end which comprises a protected or unprotected sulfhydryl, amino or hydroxyl group. Such reagents primarily react with hydroxyl groups of the oligomer. In some embodiments, such activated oligomers have a functionalizing reagent coupled to a 5′-hydroxyl group of the oligomer. In other embodiments, the activated oligomers have a functionalizing reagent coupled to a 3′-hydroxyl group. In still other embodiments, the activated oligomers have a functionalizing reagent coupled to a hydroxyl group on the backbone of the oligomer. In yet further embodiments, the oligomer is functionalized with more than one of the functionalizing reagents as described in U.S. Pat. Nos. 4,962,029 and 4,914,210, incorporated herein by reference in their entirety. Methods of synthesizing such functionalizing reagents and incorporating them into monomers or oligomers are disclosed in U.S. Pat. Nos. 4,962,029 and 4,914,210.

In some embodiments, the 5′-terminus of a solid-phase bound oligomer is functionalized with a dienyl phosphoramidite derivative, followed by conjugation of the deprotected oligomer with, e.g., an amino acid or peptide via a Diels-Alder cycloaddition reaction.

In various embodiments, the incorporation of monomers containing 2′-sugar modifications, such as a 2′-carbamate substituted sugar or a 2′-(O-pentyl-N-phthalimido)-deoxyribose sugar into the oligomer facilitates covalent attachment of conjugated moieties to the sugars of the oligomer. In other embodiments, an oligomer with an amino-containing linker at the 2′-position of one or more monomers is prepared using a reagent such as, for example, 5′-dimethoxytrityl-2′-O-(e-phthalimidylaminopentyl)-2′-deoxyadenosine-3′-N,N-diisopropyl-cyanoethoxy phosphoramidite. See, e.g., Manoharan, et al., Tetrahedron Letters, 1991, 34, 7171.

In still further embodiments, the oligomers have amine-containing functional moieties on the nucleobase, including on the N6 purine amino groups, on the exocyclic N2 of guanine, or on the N4 or 5 positions of cytosine. In some embodiments, such functionalization may be achieved by using a commercial reagent that is already functionalized in the oligomer synthesis.

Some functional moieties are commercially available, for example, heterobifunctional and homobifunctional linking moieties are available from the Pierce Co. (Rockford, Ill.). Other commercially available linking groups are 5′-Amino-Modifier C6 and 3′-Amino-Modifier reagents, both available from Glen Research Corporation (Sterling, Va.). 5′-Amino-Modifier C6 is also available from ABI (Applied Biosystems Inc., Foster City, Calif.) as Aminolink-2, and 3′-Amino-Modifier is also available from Clontech Laboratories Inc. (Palo Alto, Calif.).

In some embodiments, the compositions of the invention comprise more than one oligomer to target two or even all three target nucleic acids. In various embodiments the invention relates to a pharmaceutical composition that comprises an oligomer targeted to HER3, and an oligomer which targets and down-regulates HER2 expression. In other embodiments, which may be the same or different, the invention relates to a pharmaceutical composition comprising an oligomer targeted to HER3, and a further oligomer which targets and down-regulates EGFR expression.

In some embodiments, oligomers that target HER2 and/or EGFR mRNA (or conjugates thereof), have the same designs (e.g., gapmers, beadmers, tailmers) as oligomers that target HER3. In various embodiments, oligomers that target HER2 and/or EGFR mRNA (or conjugates thereof), have different designs from oligomers that target HER3.

In some embodiments, an oligomer for use in the compositions and methods of the invention is covalently linked to a conjugated moiety to aid in delivery of the oligomer across cell membranes. An example of a conjugated moiety that aids in delivery of the oligomer across cell membranes is a lipophilic moiety, such as cholesterol. In various embodiments, an oligomer for use in the pharmaceutical compositions of the invention is formulated with lipid formulations that form liposomes, such as Lipofectamine 2000 or Lipofectamine RNAiMAX, both of which are commercially available from Invitrogen. In some embodiments, the oligomers are formulated with a mixture of one or more lipid-like non-naturally occurring small molecules (“lipidoids”). Libraries of lipidoids can be synthesized by conventional synthetic chemistry methods and various amounts and combinations of lipidoids can be assayed in order to develop a vehicle for effective delivery of an oligomer of a particular size to the targeted tissue by the chosen route of administration. Suitable lipidoid libraries and compositions can be found, for example in Akinc et al. (2008) Nature Biotechnol., available at http://www.nature.com/nbt/journal/vaop/ncurrent/abs/nbt1402.html, which is incorporated by reference herein.

### 1.6. PROTEIN TYROSINE KINASE INHIBITORS

As used interchangeably herein, the terms “protein tyrosine kinase inhibitor,” “PTK inhibitor”, and “tyrosine kinase inhibitor” refer to molecules that bind to and inhibit the activity of one or more tyrosine kinase domains. The protein tyrosine kinase inhibitor is not the oligomer targeting HER3 as described herein. In some embodiments the protein tyrosine kinase inhibitor is a monoclonal antibody. In other embodiments the protein tyrosine kinase inhibitor is a small molecule, having a molecular weight of less than 1000 Da, such as between 300-700 Da.

In certain embodiments, the PTK inhibitors bind to and inhibit the tyrosine kinases of one or more EGFR family members. In various embodiments, the PTK inhibitors bind to and inhibit the tyrosine kinases of one or more proteins that interact with or are regulated by one or more EGFR family members, e.g., proteins involved in one or more signaling cascades that originate with one or more EGFR family members. In some embodiments, the tyrosine kinase is a receptor tyrosine kinase, i.e., is an intra-cellular domain of a larger protein that has an extra-cellular ligand binding domain and is activated by the binding of one or more ligands. In certain embodiments, the protein tyrosine kinase is a non-receptor tyrosine kinase. Tyrosine kinase enzymes regulate the activities of other proteins in one or more signaling pathways by phosphorylating them.

In various embodiments, protein tyrosine kinase inhibitors that are useful in the compositions and methods of the invention include small molecule inhibitors that bind selectively to the tyrosine kinase domain of an EGFR family member. In certain embodiments, protein tyrosine kinase inhibitors useful in the compositions and methods of the invention include small molecule inhibitors that bind to and inhibit the activity of the tyrosine kinase domains of more than one member of the EGFR family of proteins. In other embodiments, protein tyrosine kinase inhibitors useful in the compositions and methods of the invention include PTK inhibitors that do not bind selectively to the EGFR family of receptor tyrosine kinases, but also bind to the tyrosine kinase domains of other families of proteins such as VEGFR, PDGFR, and/or Raf. In certain embodiments, the PTK inhibitors are reversible inhibitors, i.e., they bind to but do not irreversibly alter the protein. In various embodiments, the PTK inhibitors are irreversible inhibitors, i.e., they inhibit PTKs by covalently crosslinking a PTK receptor dimer.

In various embodiments, the invention encompasses pharmaceutical compositions comprising a pharmaceutically acceptable derivative of a protein tyrosine kinase inhibitor. The phrase “pharmaceutically acceptable derivative,” as used herein, includes any pharmaceutically acceptable salt, prodrug, radiolabeled faint, stereoisomer, enantiomer, diastereomer, other stereoisomeric form, racemic mixture, geometric isomer, tautomer, solvate (e.g., hydrates), amorphous solid forms and crystalline solid forms of PTK inhibitors. In one embodiment, the pharmaceutically acceptable derivative is a pharmaceutically acceptable salt, radiolabeled form, stereoisomer, enantiomer, diastereomer, other stereoisomeric form, racemic mixture, geometric isomer, and/or tautomer of PTK inhibitors. In another embodiment, the pharmaceutically acceptable derivative is a pharmaceutically acceptable salt of a PTK inhibitor.

In certain embodiments, the PTK inhibitors used in the compositions and methods of the invention are in a non-salt form (e.g., in the form of a free acid or free base). In other embodiments, the PTK inhibitors used in the compositions and methods of the invention are in the form of a pharmaceutically acceptable salt. A “pharmaceutically acceptable salt” as used herein refers to salts that retain the desired biological activity and exhibit acceptable levels of undesired toxic effects.

Pharmaceutically acceptable salt forms of tyrosine kinase inhibitors can be prepared by conventional methods. If the PTK inhibitor contains an acid group, a suitable salt can be formed by reacting the compound with a suitable base to give the corresponding base-addition salt. Such bases include, but are not limited to, alkali metal hydroxides, including potassium hydroxide, sodium hydroxide and lithium hydroxide; alkaline-earth metal hydroxides, such as barium hydroxide and calcium hydroxide; alkali metal alkoxides, for example potassium ethoxide and sodium propoxide; and various organic bases, such as piperidine, diethanolamine and N-methylglutamine.

Alternatively, acid-addition salts of PTK inhibitors can be formed by treating the compounds with pharmaceutically acceptable organic and inorganic acids, for example hydrogen halides, such as hydrogen chloride, hydrogen bromide or hydrogen iodide, other mineral acids and corresponding salts thereof, such as sulfate, nitrate or phosphate and the like, and alkyl- and monoarylsulfonates, such as ethanesulfonate, toluenesulfonate and benzenesulfonate, and other organic acids and corresponding salts thereof, such as acetate, trifluoroacetate, tartrate, maleate, succinate, citrate, benzoate, salicylate, ascorbate and the like. Accordingly, pharmaceutically acceptable acid-addition salts of PTK inhibitors include but are not limited to acetate, adipate, alginate, arginate, aspartate, benzoate, benzenesulfonate (besylate), bisulfate, bisulfite, bromide, butyrate, camphorate, camphorsulfonate, caprylate, chloride, chlorobenzoate, citrate, cyclopentanepropionate, digluconate, dihydrogenphosphate, dinitrobenzoate, dodecylsulfate, ethanesulfonate, fumarate, galacterate (from mucic acid), galacturonate, glucoheptanoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, isobutyrate, lactate, lactobionate, malate, maleate, malonate, mandelate, metaphosphate, methanesulfonate, methylbenzoate, monohydrogenphosphate, 2-naphthalenesulfonate, nicotinate, nitrate, oxalate, oleate, palmoate, pectinate, persulfate, phenylacetate, 3-phenylpropionate, phosphate, phosphonate, phthalate.

PTK inhibitors useful in the methods and compositions of the invention include, but are not limited to, gefitinib (ZD-1839, Iressa®), erlotinib (OSI-1774, Tarceva™), canertinib (CI-1033), vandetanib (ZD6474, Zactima®), tyrphostin AG-825 (CAS 149092-50-2), lapatinib (GW-572016), sorafenib (BAY43-9006), AG-494 (CAS 133550-35-3), RG-13022 (CAS 149286-90-8), RG-14620 (CAS 136831-49-7), BIBW 2992 (Tovok), tyrphostin 9 (CAS 136831-49-7), tyrphostin 23 (CAS 118409-57-7), tyrphostin 25 (CAS 118409-58-8), tyrphostin 46 (CAS 122520-85-8), tyrphostin 47 (CAS 122520-86-9), tyrphostin 53 (CAS 122520-90-5), butein (1-(2,4-dihydroxyphenyl)-3-(3,4-dihydroxyphenyl)-2-propen-1-one 2′,3,4,4′-Tetrahydroxychalcone; CAS 487-52-5), curcumin ((E,E)-1,7-bis(4-Hydroxy-3-methoxyphenyl)-1,6-heptadiene-3,5-dione; CAS 458-37-7), N4-(1-Benzyl-1H-indazol-5-yl)-N6,N6-dimethyl-pyrido-[3,4-d]-pyrimidine-4,6-diamine (202272-68-2), AG-1478, AG-879, Cyclopropanecarboxylic acid-(3-(6-(3-trifluoromethyl-phenylamino)-pyrimidin-4-ylamino)-phenyl)-amide (CAS 879127-07-8), N8-(3-Chloro-4-fluorophenyl)-N2-(1-methylpiperidin-4-yl)-pyrimido[5,4-d]pyrimidine-2,8-diamine, 2HCl (CAS 196612-93-8), 4-(4-Benzyloxyanilino)-6,7-dimethoxyquinazoline (CAS 179248-61-4), N-(4-((3-Chloro-4-fluorophenyl)amino)pyrido[3,4-d]pyrimidin-6-yl)2-butynamide (CAS 881001-19-0), EKB-569, HKI-272, and HKI-357.

In some embodiments, the PTK inhibitor is selected from gefitinib, erlotinib, lapatinib, canertinib and sorafenib.

In certain embodiments, the tyrosine kinase inhibitor is gefitinib.

PTK inhibitors can be obtained by any method known in the art. In some embodiments, PTK inhibitors are available commercially from, e.g., Sigma-Aldrich®, and Cayman Chemical. In various embodiments, PTK inhibitors are available by prescription from, e.g., AstraZeneca, Roche, GlaxoSmithKline and Bayer Pharmaceuticals. In other embodiments, PTK inhibitors can be synthesized by methods known in the art, for example by methods set forth in Rewcastle, G. W. et al. (1996) J. Med. Chem. 39:918-928.

In various embodiments, the compositions of the invention comprise more than one tyrosine kinase inhibitor. In some embodiments, one tyrosine kinase inhibitor is selective for a particular receptor tyrosine kinase (e.g., gefitinib), and a second tyrosine kinase inhibitor is relatively non-selective (e.g., sorafenib). In various embodiments, a second tyrosine kinase inhibitor binds to the tyrosine kinase domains of more than one EGFR family member (e.g., lapatinib). In still further embodiments, a second tyrosine kinase inhibitor binds to the tyrosine kinase domain of a PTK receptor in a different family, such as VEGFR.

1.6.1. Pharmaceutically Acceptable Excipients and Dosage Forms

In some embodiments, the pharmaceutical compositions of the invention comprise at least one oligomeric compound, at least one PTK inhibitor or a pharmaceutically acceptable derivative thereof, and a suitable amount of a pharmaceutically acceptable excipient so as to provide the form for proper administration to a patient. As used herein, the term “patient” includes, but is not limited to, a human or a non-human animal, such as a companion animal or livestock, e.g., a cow, monkey, baboon, chimpanzee, horse, sheep, pig, chicken, turkey, quail, cat, dog, mouse, rat, rabbit or guinea pig. In various embodiments, the at least one oligomeric compound and the at least one PTK inhibitor are in a single pharmaceutical composition. In other embodiments, the at least one oligomeric compound and the at least one PTK inhibitor are in separate pharmaceutical compositions. In such embodiments where the active ingredients are in separate compositions, the compositions can be packaged together (co-packaged) for use in HER3-targeted combination therapy.

The pharmaceutical excipient can be a diluent, suspending agent, solubilizer, binder, disintegrant, preservative, coloring agent, lubricant, and the like. The pharmaceutical excipient can be a liquid, such as water or an oil, including those of petroleum, animal, vegetable, or synthetic origin, such as peanut oil, soybean oil, mineral oil, sesame oil, and the like. The pharmaceutical excipient can be saline, gum acacia, gelatin, starch paste, talc, keratin, colloidal silica, urea, and the like. In addition, auxiliary, stabilizing, thickening, lubricating, and coloring agents can be used. In one embodiment, the pharmaceutically acceptable excipient is sterile when administered to a patient. Water is a particularly useful excipient when an oligomer or PTK inhibitor is administered intravenously. Saline solutions and aqueous dextrose and glycerol solutions can also be employed as liquid excipients, particularly for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. The invention compositions, if desired, can also contain minor amounts of wetting or emulsifying agents, or pH buffering agents. Specific examples of pharmaceutically acceptable excipients that can be used to formulate oral dosage forms are described in the Handbook of Pharmaceutical Excipients, American Pharmaceutical Association (1986).

The pharmaceutical compositions of the invention can be in the form of solutions, suspensions, emulsions, tablets, pills, pellets, capsules, capsules containing liquids, powders, sustained release formulations, suppositories, emulsions, aerosols, sprays, suspensions, or any other form suitable for use. Other examples of suitable pharmaceutical excipients are described in Remington's Pharmaceutical Sciences 1447-1676 (Alfonso R. Gennaro ed., 19th ed. 1995), incorporated herein by reference.

In various embodiments, the compositions are formulated in accordance with routine procedures as a composition adapted for oral administration to humans. An oligomer or a small molecule PTK inhibitor to be orally delivered can be in the form of tablets, capsules, gelcaps, caplets, lozenges, aqueous or oily solutions, suspensions, granules, powders, emulsions, syrups, or elixirs, for example. When an active agent is incorporated into oral tablets, such tablets can be compressed tablets, tablet triturates (e.g., powdered or crushed tablets), enteric-coated tablets, sugar-coated tablets, film-coated tablets, multiply compressed tablets or multiply layered tablets. Techniques and compositions for making solid oral dosage forms are described in Pharmaceutical Dosage Forms: Tablets (Lieberman, Lachman and Schwartz, eds., 2nd ed.) published by Marcel Dekker, Inc. Techniques and compositions for making tablets (compressed and molded), capsules (hard and soft gelatin) and pills are also described in Remington's Pharmaceutical Sciences 1553-1593 (Arthur Osol, ed., 16th ed., Mack Publishing, Easton, Pa. 1980).

Liquid oral dosage forms include aqueous and nonaqueous solutions, emulsions, suspensions, and solutions and/or suspensions reconstituted from non-effervescent granules, optionally containing one or more suitable solvents, preservatives, emulsifying agents, suspending agents, diluents, sweeteners, coloring agents, flavoring agents, and the like. Techniques and composition for making liquid oral dosage forms are described in Pharmaceutical Dosage Forms: Disperse Systems, (Lieberman, Rieger and Banker, eds.) published by Marcel Dekker, Inc.

When the compositions of the invention are to be injected parenterally, they can be, e.g., in the form of an isotonic sterile solution. Alternatively, when the compositions are to be inhaled, they can be formulated into a dry aerosol or can be formulated into an aqueous or partially aqueous solution.

An orally administered composition can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, a tablet or pill form of the pharmaceutical compositions can be coated to delay disintegration and absorption in the gastrointestinal tract thereby providing a sustained action over an extended period of time. Selectively permeable membranes surrounding an osmotically active driving compound are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

In another embodiment, the compositions can be formulated for intravenous administration. Typically, compositions for intravenous administration comprise sterile isotonic aqueous buffer. Where necessary, the compositions can also include a solubilizing agent. The compositions for intravenous administration can optionally include a local anesthetic such as benzocaine or prilocaine to lessen pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where a composition is to be administered by infusion, it can be dispensed, for example, with an infusion bottle containing sterile pharmaceutical grade water or saline. Where an active agent is administered by injection, an ampule of sterile water for injection or saline can be provided so that the ingredients can be mixed prior to administration.

The pharmaceutical compositions of the invention can be administered by controlled-release or sustained-release means or by delivery devices that are known to those in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566, each of which is incorporated herein by reference. Such dosage forms can be used to provide controlled or sustained-release of one or more active ingredients using, for example, hydroxypropylmethyl cellulose, other polymer matrices, gels, permeable membranes, osmotic systems, multilayer coatings, microparticles, multiparticulates, liposomes, microspheres, or a combination thereof to provide the desired release profile in varying proportions. Suitable controlled or sustained-release formulations known to those in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled or sustained-release.

Administration of pharmaceutical compositions described herein may be oral, pulmonary, topical (e.g., epidermal, transdermal, ophthalmic and mucous membranes including vaginal and rectal delivery), or parenteral including intravenous, intraarterial, subcutaneous, intraperitoneal or intramuscular injection or infusion. In one embodiment, a pharmaceutical composition containing therapeutic oligomers is administered intravenously (i.v.), intraperitoneally (i.p.) or as a bolus injection. Parenteral routes are preferred in many aspects of the invention. Proper formulation is dependent upon the route of administration chosen, i.e. whether local or systemic treatment is treated. In various embodiments where the at least one oligomer and the at least one PTK inhibitor are formulated in separate compositions, the pharmaceutical compositions need not be of the same form (e.g., solid dosage form, liquid dosage form, aerosol) and need not be administered by the same route (e.g., orally, parenterally, topically) or at the same time. For example, the invention encompasses pharmaceutical compositions wherein the oligomer is formulated in a dosage form for oral administration, e.g., a tablet, capsule, oral syrup and the like, and wherein the PTK inhibitor is formulated in a dosage form for intravenous administration or administration by inhalation.

1.6.2. Dosage Regimens

The LNA oligomer targeting HER3 (and optionally one or more of HER2 and EFGR) can be administered at regular intervals (“dose intervals” or “DI”) ranging from 3 days to two weeks. In some embodiments, the DI is 4, 5, 6, 7, 8, 9, 0, 11, 12, or 13 days. In various embodiments, the DI is about 1 week. In still further embodiments, the DI is 6, 7 or 8 days. Suitably at least two doses are provided with a DI between the two doses, such as 3, 4, 5, 6, 7, 8, 9 or 10 doses, each with a DI between successive doses of LNA oligomer. The DI period between each dose may the same. In some embodiments, the DI period ranges from 3 days to two weeks. In other embodiments, the DI period is 4, 5, 6, 7, 8, 9, 10, 11, 12, or 13 days. In still other embodiments, the DI period is about 1 week. In certain embodiments, the DI period is 6, 7 or 8 days.

In some embodiments, each dose of the LNA oligomer targeting HER3 (and optionally one or more of HER2 and EGFR) ranges from about 0.25 mg/kg to about 10 mg/kg of body weight, such as about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, or about 9 mg/kg. In some embodiments, each dose of the LNA oligomer targeting HER3 (and optionally one or more of HER2 and EGFR) ranges from about 2 mg/kg to about 8 mg/kg, or from about 4 to about 6 mg/kg, or from about 4 mg/kg to about 5 mg/kg. In some embodiments, each dose of the LNA oligomer targeting HER3 (and optionally one or more of HER2 and EGFR) is at least 2 mg/kg, such as 2, 3, 4, 5, 6, 7 or 8 mg/kg. In various embodiments, each dose is 6 mg/kg.

Administration of the LNA oligomer is typically performed by parenteral administration, such as subcutaneous, intramuscular, intravenous or intra peritoneal administration. In certain embodiments, administration is intravenous.

In some embodiments the dosage regimen for the LNA oligomer is repeated after an initial dosage regimen. In various embodiments, the dosage regimen is repeated as necessary in order to treat or prevent the progression of the disease.

In certain embodiments, LNA oligomers targeting HER3 (and optionally one or more of HER2 and EGFR) are administered over a relatively short time period rather than continuously. In various embodiments, a short administration time provides a marked improvement in the quality of life for the patient, as he is not required to be hospital bound for long periods of time. Therefore in various embodiment, the LNA oligomer targeting HER3 (and optionally one or more of HER2 and EGFR) is not administered by continuous infusion. Each dose of the LNA oligomer can therefore be administered to the patient in a time period of less than 12 hours, such as less than about 8 hours, less than about 4 hours, such as less than about 3 hours. Each dose of the LNA oligomer may therefore be administered to the patient in a time period ranging from about 1 hour and about 4 hours, such as from about 2 hours and about 3 hours, or about 2 hours. The LNA oligomer can be administered to the patient in a time period of at least 30 minutes such as at least 1 hour. Such administrations can be given, e.g., intravenously.

A pharmaceutically effective dose of the protein tyrosine kinase inhibitor can, in some embodiments can be administered prior to, concurrently with or subsequently to the administration of one or more pharmaceutically effective doses of the LNA oligomer targeting HER3 (and optionally one or more of HER2 and EGFR). Typically, one or more effective doses of the protein tyrosine kinase inhibitor is administered so that the both the LNA oligomer and the protein tyrosine kinase provide concurrent therapeutic benefits to the patient.

1.6.3. Kits

The invention also provides a kit comprising a first component and a second component. In various embodiments, the first component comprises at least one oligomer that is capable of inhibiting (e.g., by down-regulating) expression of HER3, or a conjugate and/or pharmaceutical composition thereof, and the second component comprises at least one small molecule protein tyrosine kinase inhibitor that is selective for one or more EGFR family members. In other embodiments, the kit comprises a third component which is a therapeutic agent other than an oligonucleotide or a PTK inhibitor, such as a chemotherapeutic agent (e.g., taxol). In some embodiments, kits of the invention are used in methods of treating a hyperproliferative disorder, such as cancer, which comprises administering to a patient in need thereof an effective amount of a first component and a second component of the kit. In various embodiments, the first and second components are administered concurrently or simultaneously. In other embodiments, the first and second components are administered sequentially and in any order.

In some embodiments, the kit comprises a first component that comprises an oligomer of the invention that is capable of inhibiting (e.g., by down-regulating) expression of HER3, or a conjugate and/or pharmaceutical composition thereof, and a second component that is a protein tyrosine kinase inhibitor and a third component that is an oligomer capable of inhibiting (e.g., by down-regulating) the expression of one or more of HER2 and EGFR as described herein, or a conjugate and/or pharmaceutical composition thereof.

One embodiment of the invention provides a kit that includes the at least one oligomeric compound and the at least one PTK inhibitor, in separate compositions within the kit. For example, one kit embodiment of the invention comprises an oligomeric compound according to SEQ ID NO: 180 and the PTK inhibitor gefitinib, each as separate compositions within the kit.

### 1.7. METHODS

In certain embodiments, the invention encompasses methods of inhibiting the expression and/or activity of HER3 in a cell, comprising contacting the cell with an effective amount of an oligomeric compound (or a conjugate thereof) and an effective amount of a protein tyrosine kinase inhibitor so as to effect the inhibition (e.g., down-regulation) of HER3 (and optionally one or more of HER2 and EGFR) expression and/or activity in a cell. In certain embodiments, HER3 (and optionally one or more of HER2 and EGFR) mRNA expression is inhibited. In other embodiments, HER3 (and optionally one or more of HER2 and EGFR) protein expression is inhibited. In still other embodiments, the activity of the tyrosine kinase of an EGFR family member is inhibited (e.g., down-regulated). In various embodiments, the internalization of HER3 (and optionally of one or more of HER2 and EGFR) into the cell is inhibited (e.g., down-regulated). In various embodiments, the cell is a mammalian cell, such as a human cell.

In certain embodiments, the contacting occurs in vitro. In other embodiments, the contacting is effected in vivo by administering the compositions of the invention to a mammal. In various embodiments, the invention provides a method of inhibiting (e.g., by down-regulating) the expression of HER3 protein and/or mRNA, and/or the internalization of HER3 into a cell, and the expression of HER2 protein and/or mRNA in a cell and/or the activity of the HER2 tyrosine kinase, and/or the internalization of HER2 into a cell. The sequence of the human HER2 mRNA is shown in SEQ ID NO: 199. In still further embodiments, the invention provides a method of inhibiting (e.g., by down-regulating) the expression of HER3 protein and/or mRNA in a cell, and/or the internalization of HER3 into a cell, and the expression of EGFR protein and/or mRNA in a cell, and/or the activity of the EGFR tyrosine kinase, and/or the internalization of EGFR into a cell. The sequence of the human EGFR mRNA is shown in SEQ ID NO: 198. In yet further embodiments, the invention provides a method of inhibiting (e.g., by down-regulating) the expression of HER3, HER2 and EGFR mRNA and/or protein in a cell, and/or the activity of HER2 and EGFR tyrosine kinases, and/or the internalization of HER3, HER2 and EGFR into a cell.

In certain embodiments, the invention relates to a method of treating a disease in a patient, comprising administering to a patient in need thereof a pharmaceutical composition comprising an effective amount of at least one oligomer, or a conjugate thereof, an effective amount of at least one small molecule protein tyrosine kinase inhibitor and a pharmaceutically acceptable excipient. As used herein, the terms “treating” and “treatment” refer to both treatment of an existing disease (e.g., a disease or disorder as referred to herein below), or prevention of a disease, i.e., prophylaxis.

In various embodiments, the invention relates to a method of treating a disease in a patient, wherein the oligomer (or conjugate thereof) and the protein tyrosine kinase inhibitor are in different pharmaceutical compositions. In certain embodiments, the two compositions can be administered concurrently or simultaneously. In other embodiments, the two compositions can be administered sequentially in any order. In various embodiments, the composition comprising the oligonucleotide (or conjugate thereof) and the composition comprising the protein tyrosine kinase inhibitor can be administered with different dosing schedules and in different concentrations, in different dosage forms, and by different routes of administration.

Methods of administration include, but are not limited to, intradermal, intramuscular, intraperitoneal, parenteral, intravenous, subcutaneous, intranasal, epidural, oral, sublingual, intracerebral, intravaginal, transdermal, rectal, by inhalation, or topical, particularly to the ears, nose, eyes, or skin. The method of administration is left to the discretion of the practitioner.

Pulmonary administration can also be employed, e.g., by use of an inhaler or nebulizer, and formulation with an aerosolizing agent, or via perfusion in a fluorocarbon or synthetic pulmonary surfactant. In certain embodiments, an oligomer (or conjugate thereof) and/or a protein tyrosine kinase inhibitor can be formulated as a suppository, with traditional binders and excipients such as triglycerides.

When an oligomer (or conjugate thereof) and/or a protein tyrosine kinase inhibitor is incorporated for parenteral administration by injection (e.g., continuous infusion or bolus injection), the formulation for parenteral administration can be in the form of a suspension, solution, emulsion in an oily or aqueous vehicle, and such formulations can further comprise pharmaceutically necessary additives such as one or more stabilizing agents, suspending agents, dispersing agents, and the like. An oligomer (or conjugate thereof) and/or protein tyrosine kinase inhibitor can also be in the form of a powder for reconstitution as an injectable formulation.

In other embodiments, an oligomer (or conjugate thereof) and/or protein tyrosine kinase inhibitor can be delivered in a vesicle, in particular a liposome (see Langer, Science 249:1527-1533 (1990); and Treat et al., Liposomes in the Therapy of Infectious Disease and Cancer 317-327 and 353-365 (1989)).

In yet other embodiments, an oligomeric compound (or conjugate thereof) and/or a protein tyrosine kinase inhibitor can be delivered in a controlled-release system or sustained-release system (see, e.g., Goodson, “Dental Applications” (pp. 115-138) in Medical Applications of Controlled Release, Vol. 2, Applications and Evaluation, R. S. Langer and D. L. Wise eds., CRC Press (1984); Langer, Science 249:1527-1533 (1990)). In various embodiments, controlled-release or sustained-release delivery can be effected by a pump (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J. Med. 321:574 (1989)), or with the use of polymeric materials (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 23:61 (1983); Levy et al., Science 228:190 (1985); During et al., Ann. Neurol. 25:351 (1989); and Howard et al., J. Neurosurg. 71:105 (1989)).

In certain embodiments, the compositions of the invention are useful for inhibiting cell proliferation. In various embodiments the anti-proliferative effect is an at least 10% reduction, an at least 20% reduction, an at least 30% reduction, an at least 40% reduction, an at least 50% reduction, an at least 60% reduction, an at least 70% reduction, an at least 80% reduction, or an at least 90% reduction in cell proliferation as compared to a cell sample that is untreated. In other embodiments, the anti-proliferative effect is an at least 10% reduction, an at least 20% reduction, an at least 30% reduction, an at least 40% reduction, an at least 50% reduction, an at least 60% reduction, an at least 70% reduction, an at least 80% reduction, or an at least 90% reduction in cell proliferation as compared to a cell sample that is treated with either an oligomeric compound or a small molecule protein tyrosine kinase inhibitor alone (“monotherapy”). In various embodiments, the cell is a cancer cell. In some embodiments, the cancer cell is selected from a breast cancer cell, a prostate cancer cell, a lung cancer cell, and an epithelial carcinoma cell.

Accordingly, the compositions of the invention are useful for treating a hyperproliferative disease, such as cancer. In some embodiments, the cancer to be treated by the HER3-targeted combination therapy of the invention is selected from the group consisting of lymphomas and leukemias (e.g. non-Hodgkin's lymphoma, Hodgkin's lymphoma, acute leukemia, acute lymphocytic leukemia, acute myelocytic leukemia, chronic myeloid leukemia, chronic lymphocytic leukemia, multiple myeloma), colon carcinoma, rectal carcinoma, epithelial carcinoma, pancreatic cancer, breast cancer, ovarian cancer, prostate cancer, renal cell carcinoma, hepatoma, bile duct carcinoma, choriocarcinoma, cervical cancer, testicular cancer, lung carcinoma, bladder carcinoma, melanoma, head and neck cancer, brain cancer, cancers of unknown primary site, neoplasms, cancers of the peripheral nervous system, cancers of the central nervous system, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumour, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, seminoma, embryonal carcinoma, Wilms' tumour, small cell lung carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, and retinoblastoma, heavy chain disease, metastases, or any disease or disorder characterized by uncontrolled or abnormal cell growth.

In certain embodiments, the disease is a cancer selected from the group consisting of lung cancer, prostate cancer, breast cancer, ovarian cancer, colon cancer, epithelial carcinoma, and stomach cancer.

In certain other embodiments, the lung cancer is non-small cell lung cancer.

As shown in the Example below, the combination therapy regimens of the invention allow for the treatment of cancers that are resistant to monotherapy, e.g., with gefitinib or another PTK inhibitor.

In various embodiments, the treatment of a disease according to the invention may be combined with one or more other anti-cancer treatments, such as radiotherapy, chemotherapy or immunotherapy.

In certain embodiments, the disease is associated with a mutation in the HER3 gene (and/or the HER2 gene and/or the EGFR gene) or a gene whose protein product is associated with or interacts with HER3. In some embodiments, the mutated gene codes for a protein with a mutation in the tyrosine kinase domain. In various embodiments, the mutation in the tyrosine kinase domain is in the binding site of a small molecule PTK inhibitor and/or the ATP binding site. Therefore, in various embodiments, the target mRNA is a mutated form of the HER3 (and/or HER2 and/or EGFR) sequence; for example, it comprises one or more single point mutations, such as SNPs associated with cancer.

In certain embodiments, the disease is associated with abnormal levels of a mutated form of HER3. In certain embodiments, the disease is associated with abnormal levels of a wild-type form of HER3. One aspect of the invention is directed to a method of treating a patient suffering from or susceptible to conditions associated with abnormal levels of HER3, comprising administering to the patient a therapeutically effective amount of an oligomer targeted to HER3 or a conjugate thereof, and an effective amount of a small molecule protein tyrosine kinase inhibitor that binds to the tyrosine kinase domain of an EGFR family member and/or of a protein that interacts with one or more EGFR family members. In some embodiments, the oligomer comprises one or more LNA units. In various embodiments the PTK inhibitor is gefitinib.

In another embodiment, the invention is directed to a method of treating a patient suffering from or susceptible to conditions associated with abnormal levels of a mutated form of HER2, or abnormal levels of a wild-type form of HER2, comprising administering to the mammal a therapeutically effective amount of an oligomer targeted to HER3 (and optionally to one or more of HER2 and EGFR) or a conjugate thereof, and an effective amount of a small molecule tyrosine kinase inhibitor that binds to the tyrosine kinase domain of one or more EGFR family members and/or of a protein that interacts with one or more EGFR family members. In some embodiments, the oligomer comprises one or more LNA units. In various embodiments the PTK inhibitor is gefitinib.

In still other embodiments, the invention is directed to a method of treating a patient suffering from or susceptible to conditions associated with abnormal levels of a mutated EGFR, or abnormal levels of a wild-type EGFR, comprising administering to the patient a therapeutically effective amount of an oligomer targeted to HER3 (and optionally to one or more of HER2 and EGFR) or a conjugate thereof, and an effective amount of a small molecule tyrosine kinase inhibitor that binds to the tyrosine kinase domain of an EGFR family member and/or of a protein that interacts with one or more EGFR family members. In some embodiments, the oligomer comprises one or more LNA units. In various embodiments the PTK inhibitor is gefitinib.

In various embodiments, the invention described herein encompasses a method of preventing or treating a disease comprising administering to a human in need of such therapy a therapeutically effective amount an oligomer that modulates HER3 modulating oligomer (and optionally one or more of HER2 and EGFR) or a conjugate thereof, and an effective amount of a tyrosine kinase inhibitor that binds to the tyrosine kinase domain of and EGFR family member and/or of a protein that interacts with one or more EGFR family members.

The amount of the at least one oligomer and of the at least one PTK inhibitor that is effective for the treatment or prevention of a disease can be determined by standard clinical techniques. Generally the dosage ranges can be estimated based on EC₅₀ found to be effective in in vitro and in vivo animal models. The precise doses to be employed will also depend on, e.g., the routes of administration and the seriousness of the disease, and can be decided according to the judgment of a practitioner and/or each patient's circumstances. In other examples thereof, variations will necessarily occur depending upon, inter alia, the weight and physical condition (e.g., hepatic and renal function) of the patient being treated, the affliction to be treated, the severity of the symptoms, the frequency of the dosage interval, the presence of any deleterious side-effects, and the particular oligonucleotide and PTK inhibitor utilized.

In various embodiments, the dosage of an oligomer is from about 0.01 μg to about 1 g per kg of body weight, and may be given once or more daily, weekly, monthly or yearly, or even once every 2 to 10 years or by continuous infusion for hours up to several months. In certain embodiments, the dosage of a PTK inhibitor is from about 50 mg to about 500 mg per day. In various embodiments, the dosage of a PTK inhibitor is from about 100 mg to about 400 mg per day. In other embodiments, the dosage of a PTK inhibitor is from about 150 mg to about 300 mg per day. In certain embodiments, repetition rates for dosing can be estimated based on measured residence times and concentrations of the active agents in bodily fluids or tissues. Following successful treatment, the patient can undergo maintenance therapy with the HER3-targeted combination therapy to prevent the recurrence of the disease state.

### 1.8. EXAMPLES

### Example 1

### ErbB-3 (HER3)-Targeted Combination Therapy Decreases Cancer Cell Proliferation

### Experimental Procedures

1. Cell Culture

The combination effects of the oligomer having the base sequence and design as set forth in SEQ ID NO: 180 (hereinafter referred to as “ON180”) with gefitinib, an EGFR inhibitor, were examined in several tumor cell lines. Cells were cultured in the medium as described below and maintained at 37° C. at 95% humidity and 5% CO₂. Cells were routinely passaged 2-3 times weekly.

15PC-3 (Santaris Pharma): The human prostate cancer cell line 15PC-3 was cultured in DMEM (ATCC)+10% fetal bovine serum (FBS)+2 mM Glutamax™ I+gentamicin (25 μg/ml).
A549 (ATCC): The human lung cancer cell line A549 was cultured in F12K Medium (ATCC)+10% FBS+2 mM Glutamax™ I+Penicillin (100 u/ml)/Streptomycin (100 μg/ml).
DU145 (ATCC): The human prostate cancer cell line DU145 was cultured in Eagle's Minimum Essential Medium (ATCC)+10% FBS+2 mM Glutamax™ I+Penicillin (100 u/ml)/Streptomycin (100 μg/ml).
A431 (ATCC): The human epidermoid cancer cell line A431 was cultured in DMEM (ATCC)+10% fetal bovine serum (FBS)+2 mM Glutamax™ I+Penicillin (100 u/ml)/Streptomycin (100 μg/ml).
SKBR-3 (ATCC): The human breast cancer cell line SKBR3 was cultured in McCoy's 5A Medium. Modified (ATCC)+10% FBS+2 mM Glutamax™ I+Penicillin (100 u/ml)/Streptomycin (100 μg/ml).
H1993 (ATCC): The human lung cancer cell line H1993 was cultured in RPMI-1640 (ATCC)+10% FBS+2 mM Glutamax™ I+Penicillin (100 u/ml) I Streptomycin (100 μg/ml).

2. Combined Treatment with ON180 and Gefitinib

The cells were treated with either ON180 or an LNA-containing oligonucleotide having a scrambled base sequence as set forth in SEQ ID NO: 236 (hereinafter referred to as “ONCONT”) using the cationic liposome formulation Lipofectamine™-2000 (Invitrogen™) as transfection vehicle. Cells were seeded in 6-well plates (NUNC™) and treated when 50-60% confluent. The transfection of cells by ON180 was performed as described by the manufacturer using serum-free OptiMEM® (Gibco™) and 5 μg/ml Lipofectamine™-2000. ONCONT served as a negative control. The treated cells were incubated at 37° C. for 4 hours and then washed with OptiMEM®, after which regular serum-containing medium was added.

24 hours after transfection with the oligonucleotides (ON180 or ONCONT), the cells were treated with gefitinib (Amfinecom, Inc.), a marketed EGFR inhibitor drug (1 μM to 40 μM final concentration) for 48 hours. The treated cells were then subjected to proliferation assay by MTS and ErbB3 mRNA quantitation by qRT-PCR, respectively (see below). Each experiment was performed at least two times.

3. Cell Proliferation Assay (MTS Assay)

The proliferation assay was carried out by using CellTiter 96® Aqueous One solution reagent (Promega, Cat# 358B) following the manufacturer's instructions. Briefly, the MTS compound was added to the culture of the 6-well plate, and incubated at 37° C., 95% humidity and 5% CO₂ for 1-3 hours before measurement. The medium with the MTS reagent was then transferred to 96-well plate. The absorbance was measured at 490 rim with a reference of 650 nm using an ELISA reader (Molecular Devices). The background for the assay was measured from wells containing only medium and was subtracted from the signal from the wells containing cells. The absorbance at 490 nm (OD490 nm) is proportional to the viable cell number in culture.

4. Examination of ErbB3 mRNA Level by qRT-PCR

Total RNA was extracted from the treated cells as described above, using Qiagen RNeasy Plus Mini Kit (Cat# 74134). One-step qRT-PCR was used to examine ErbB3 mRNA levels in the cells by using the QuantiTect Probe RT-PCR kit (Cat#: 204443; Qiagen) according to the manufacturer's instructions. The sequences for the primers and probes were as follows:

Human ErbB3 PCR primer/probe set:

| | | |
|---|---|---|
| Probe: | CATTGCCCAACCTCCGCGTG | (SEQ ID NO: 250) |
| Primer-1: | TGCAGTGGATTCGAGAAGTG | (SEQ ID NO: 251) |
| Primer-2: | GGCAAACTTCCCATCGTAGA | (SEQ ID NO: 252) |

Human GAPDH primer/probe set:

| | | |
|---|---|---|
| Probe: | ACTGGCGCTGCCAAGGCTGT | (SEQ ID NO: 253) |
| Primer-1: | CCACCCAGAAGACTGTGGAT | (SEQ ID NO: 254) |
| Primer-2: | TTCAGCTCAGGGATGACCTT | (SEQ ID NO: 255) |

The qRT-PCR was performed on the Applied Biosystems 7500 Fast Real-Time PCR System using 120 ng of total RNA sample. GAPDH mRNA served as an internal control.

### Results

A549 cells are resistant to gefitinib. Gefitinib alone did not affect proliferation at 40 μM in this cell line (FIG. 1A). ON180 alone potently inhibited expression of ErbB3 mRNA production (IC50<2 nM; FIG. 1C) and cell growth (IC₅₀<5 nM) (FIG. 1A, 1B). Treatment with 2 nM ON180 in combination with gefitinib significantly enhanced the anti-proliferative effect of gefitinib on A549 cells. (FIG. 1A, 1B). As demonstrated in FIG. 1B, the combination of 40 μM gefitinib and 2 nM ON180 reduced the growth rate of A549 cells by about 50% as compared with A549 cells treated with 40 μM gefitinib monotherapy.

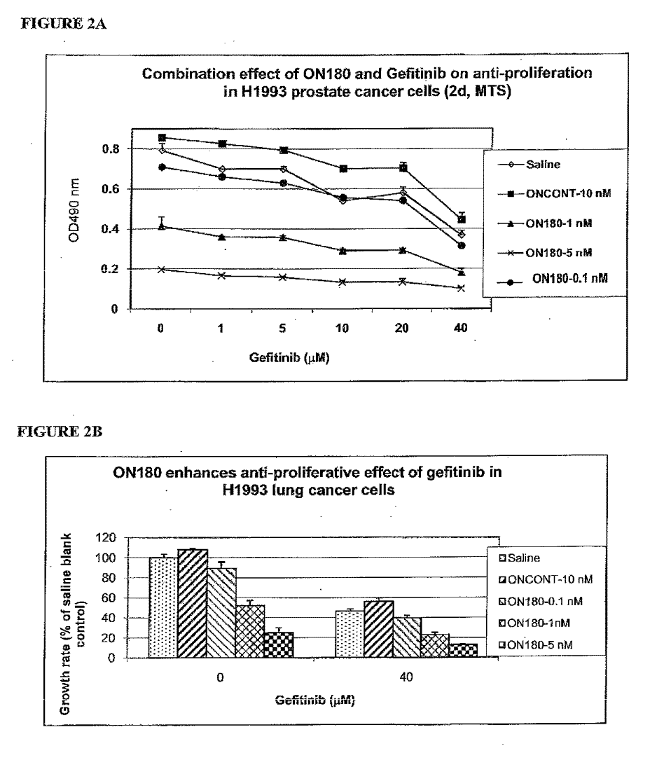

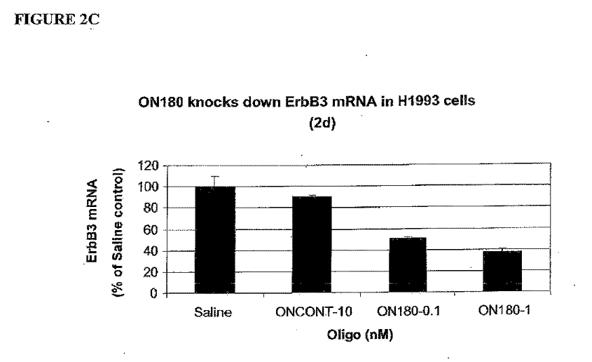

H1993 cells are relatively insensitive to gefitinib (IC₅₀ 40 nM) (FIG. 2A. ON180 alone potently inhibited expression of ErbB3 mRNA (FIG. 2C) and cell growth (IC₅₀=1 nM) (FIG. 2A, 2B). Treatment with a combination of 1 nM ON180 and gefitinib enhanced the anti-proliferative effect of gefitinib on H1993 cells (FIG. 2A, 2B). As demonstrated by FIG. 2B, the combination of 40 μM gefitinib and 1 nM ON180 reduced the growth rate of H1993 cells by more than 50% as compared with treatment with 40 μM gefitinib monotherapy.

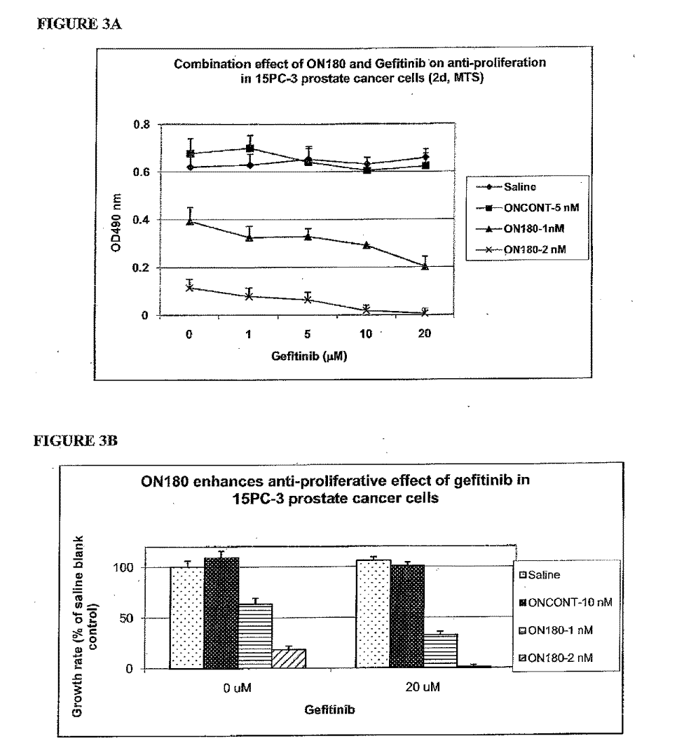

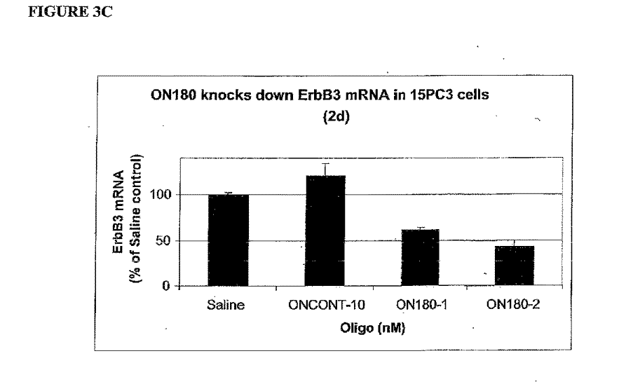

15PC3 cells are resistant to gefitinib. Gefitinib did not affect proliferation at 20 μM in this cell line. (FIG. 3A) ON180 alone potently inhibited ErbB3 mRNA (FIG. 3C) and cell growth (IC₅₀<2 nM) (FIG. 3A, 3B). Treatment of 15PC3 cells with a combination of 1 nM ON180 and 20 μM gefitinib significantly enhanced (i.e., by almost 70%) the anti-proliferative effect of gefitinib on 15PC3 cells as compared to treatment with 20 μM gefitinib monotherapy (FIG. 3A, 3B).

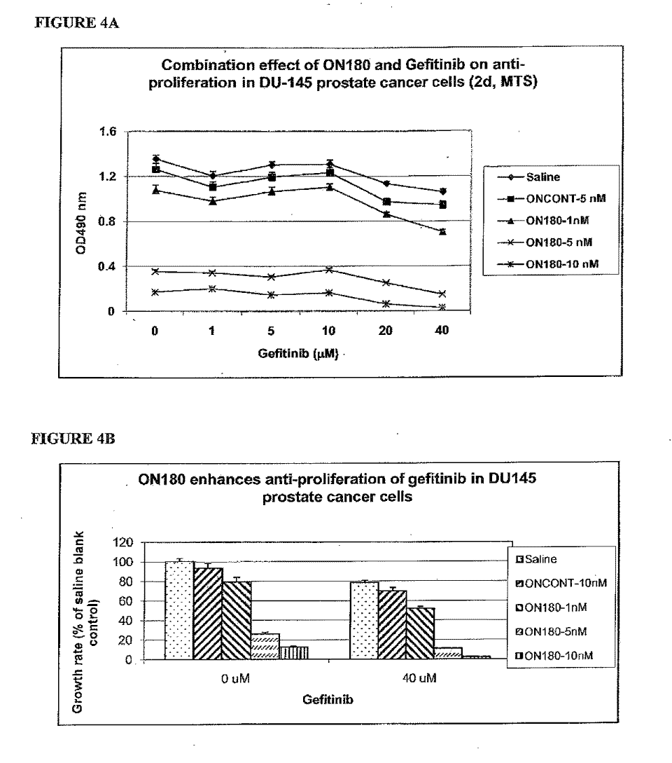

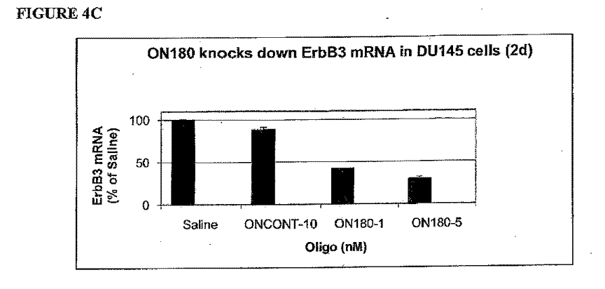

DU145 cells are resistant to gefitinib. Gefitinib did not affect proliferation at 40 μM in this cell line. (FIG. 4A) ON180 alone effectively inhibited expression of ErbB3 mRNA (FIG. 4C) and cell growth (IC₅₀<5 nM) (FIG. 4A, 4B). Treatment of DU145 cells with a combination of 1 nM ON180 and 40 μM gefitinib significantly enhanced (i.e., by about 40%) the anti-proliferative effect of gefitinib on DU145 cells as compared to treatment with 40 μM gefitinib monotherapy (FIG. 4A, 4B).

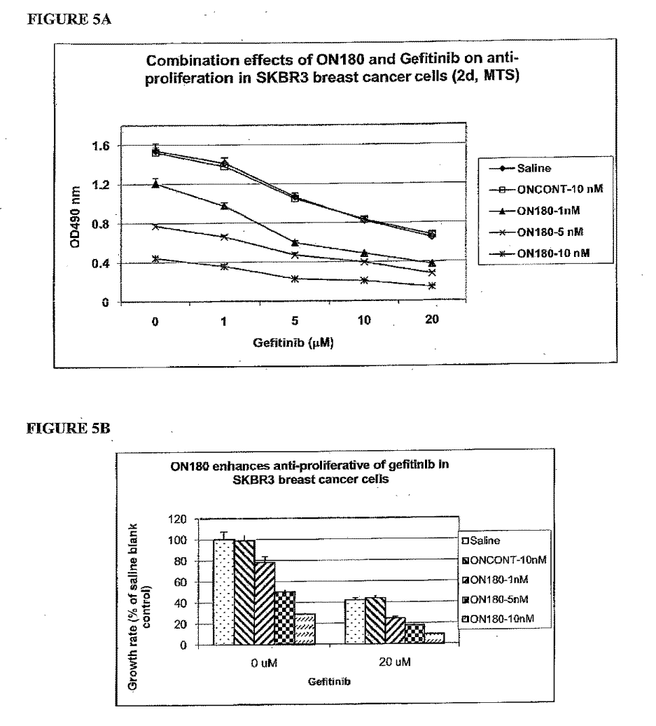

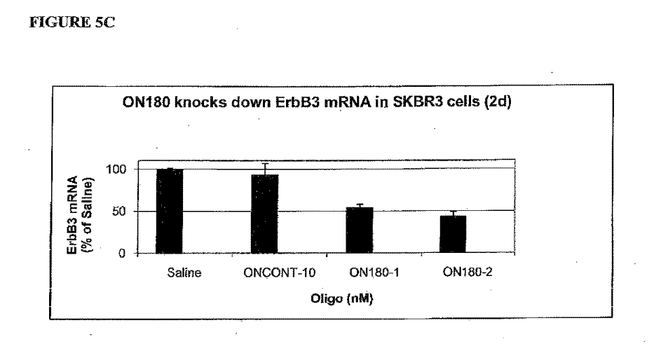

SKBR3 cells are sensitive to gefitinib. (FIG. 5A) Exposure of SKBR3 cells to ON180 alone effectively inhibited expression of ErbB3 mRNA (FIG. 5C) and cell growth (IC₅₀<5 nM) (FIG. 5A, 5B). Treatment of these tumor cells with a combination of 1 nM ON180 and 20 μM gefitinib significantly enhanced (i.e., by more than 50%) the anti-proliferative effect of gefitinib on SKBR3 cells as compared to treatment with 20 μM gefitinib monotherapy (FIG. 5A, 5B).

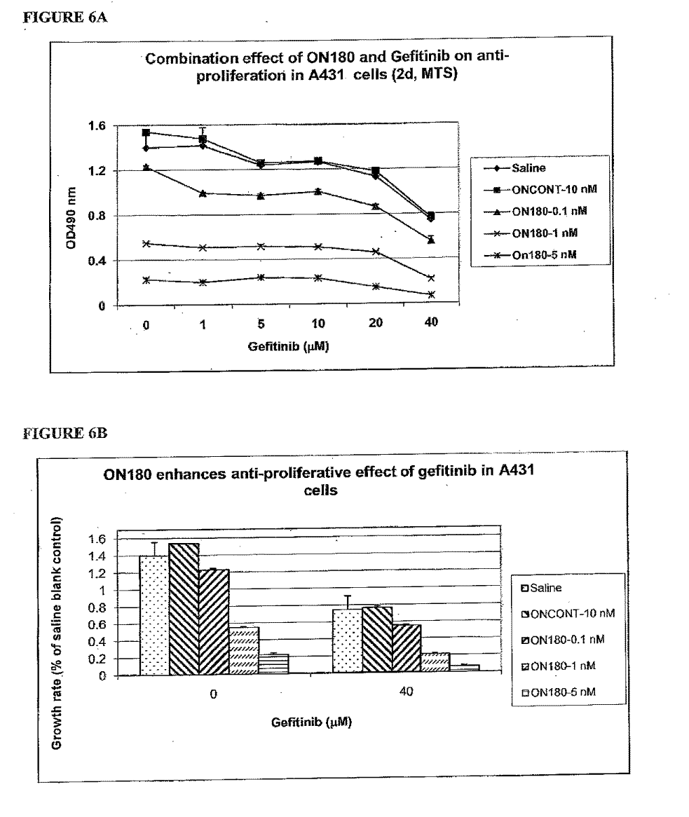

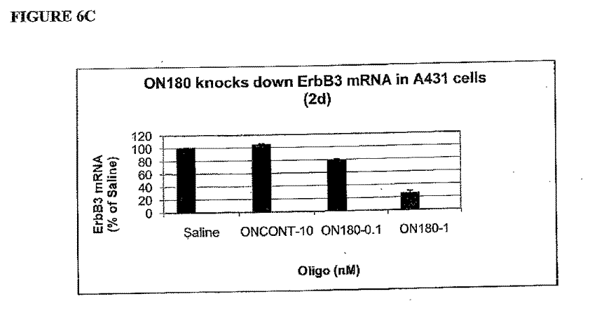

A431 cells are sensitive to gefitinib. (FIG. 6A) Exposure of these tumor cells to ON180 alone effectively inhibited ErbB3 mRNA (FIG. 6C) and cell growth (IC₅₀<1 nM) (FIG. 6A, 6B). Treatment of A431 cells with a combination of 1 nM ON180 and 40 μM gefitinib significantly enhanced (i.e., by about 50%) the anti-proliferative effect of gefitinib on A431 cells as compared to treatment with 20 μM gefitinib monotherapy (FIG. 6A, 6B).

### CONCLUSIONS

The oligomeric compound ON180 potently inhibited expression of ErbB3 mRNA and cell proliferation in the six tested cancer cell lines (A549, H1993, 15PC3, DU145, A431 and SKBR3). Two of the cell lines, SKBR3 and A431, are sensitive to gefitinib, while four, A549, H1993, 15PC3 and DU145, are insensitive or resistant to the PTK inhibitor. Nevertheless, effects on cell proliferation of treatment with a combination of ON180 and gefitinib were observed in all of the six tested tumor cell lines. ON180 treatment enhanced sensitivity of the resistant tumor cells (A549, H1993, DU145 and 15PC3) to gefitinib at low concentration (1-5 nM).

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

1. A composition comprising:
(a.) an oligomer consisting of 10 to 50 contiguous monomers wherein adjacent monomers are covalently linked by a phosphate group or a phosphorothioate group,
wherein said oligomer comprises a first region of at least 10 contiguous monomers that is at least 80% identical to the sequence of a region of at least 10 contiguous monomers present in a compound selected from the group consisting of
(SEQ ID NO: 169)
5′-Gₛ^{Me}CₛTₛCₛcₛaₛgₛaₛcₛaₛtₛcₛaₛ^{Me}CₛTₛ^{Me}C-3′;
and
(SEQ ID NO: 180)
5′-TₛAₛGₛcₛcₛtₛgₛtₛcₛaₛcₛtₛtₛ^{Me}CₛTₛ^{Me}C-3′,
wherein uppercase letters denote beta-D-oxy-LNA monomers and lowercase letters denote DNA monomers, the subscript “s” denotes a phosphorothioate linkage, and ^{Me}C denotes a beta-D-oxy-LNA monomer containing a 5-methylcytosine base, and
wherein at least one monomer of said first region is a nucleoside analogue; and
(b.) a protein tyrosine kinase inhibitor of EGFR (HER1).

2. The composition according to claim 1, wherein the protein tyrosine kinase inhibitor of EGFR (HER1) is selected from the group consisting of gefitinib, erlotinib, lapatinib and canertinib.

3-6. (canceled)

7. The composition according to claim 1, wherein each nucleoside analogue is independently selected from the group consisting of an LNA monomer, a monomer containing a 2′-O-alkyl-ribose sugar, a monomer containing a 2′-O-methyl-ribose sugar, a monomer containing a 2′-aminodeoxyribose sugar, and a monomer containing a 2′fluoro-deoxyribose sugar.

8-11. (canceled)

12. A method of treating cancer in a mammal comprising administering to said mammal the composition of claim 1, wherein the cancer is selected from the group consisting of lung cancer, prostate cancer, breast cancer, epithelial carcinoma, epidermoid carcinoma, non-Hodgkin's lymphoma, Hodgkin's lymphoma, acute leukemia, acute lymphocytic leukemia, acute myelocytic leukemia, chronic myeloid leukemia, chronic lymphocytic leukemia, multiple myeloma, colon carcinoma, rectal carcinoma, epithelial carcinoma, pancreatic cancer, ovarian cancer, renal cell carcinoma, hepatoma, bile duct carcinoma, choriocarcinoma, cervical cancer, testicular cancer, lung carcinoma, bladder carcinoma, melanoma, head and neck cancer, brain cancer, cancers of unknown primary site, neoplasms, cancers of the peripheral nervous system, cancers of the central nervous system, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumour, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, seminoma, embryonal carcinoma, Wilms' tumour, small cell lung carcinoma, glioma, astrocytoma, medulloblastoma, craniopharyngioma, ependymoma, pinealoma, hemangioblastoma, acoustic neuroma, oligodendroglioma, meningioma, neuroblastoma, and retinoblastoma.

13. (canceled)

14. A pharmaceutical composition comprising:
(a) an oligomer, or a conjugate comprising an oligomer, the oligomer consisting of 10 to 50 contiguous monomers wherein adjacent monomers are covalently linked by a phosphate group or a phosphorothioate group,
wherein said oligomer comprises a first region of at least 10 contiguous monomers;
wherein at least one monomer of said first region is a nucleoside analog;
wherein the sequence of said first region is at least 80% identical to the reverse complement of the best-aligned target region of a mammalian HER3 gene or a mammalian HER3 mRNA;
(b) a protein tyrosine kinase inhibitor; and
(c) a pharmaceutically acceptable excipient.

15. The composition according to claim 14, wherein the sequence of the first region of the oligomer is at least 80% identical to the sequence of a region of at least 10 contiguous monomers present in SEQ ID NOs: 1-140 and 169-234.

16. (canceled)

17. The composition according to claim 15, wherein the sequence of the first region of the oligomer is at least 80% identical to the sequence of a region of at least 10 contiguous monomers present in SEQ ID NOs: 169 or 180.

18. The composition according to claim 14, wherein the protein tyrosine kinase inhibitor is selected from the group consisting of gefitinib, erlotinib, canertinib, vandetanib, lapatinib, sorafenib, AG-494, RG-13022, RG-14620, BIBW 2992, tyrphostin AG-825, tyrphostin 9, tyrphostin 23, tyrphostin 25, tyrphostin 46, tyrphostin 47, tyrphostin 53, butein, curcumin, AG-1478, AG-879, cyclopropanecarboxylic acid-(3-(6-(3-trifluoromethyl-phenylamino)-pyrimidin-4-ylamino)-phenyl)-amide, N8-(3-Chloro-4-fluorophenyl)-N2-(1-methylpiperidin-4-yl)-pyrimido[5,4-d]pyrimidine-2,8-diamine, 2HCl (CAS 196612-93-8), 4-(4-benzyloxyanilino)-6,7-dimethoxyquinazoline, N-(4-((3-Chloro-4-fluorophenyl)amino)pyrido[3,4-d]pyrimidin-6-yl)-2-butynamide (CAS 881001-19-0), EKB-569, HKI-272, and HKI-357.

19. (canceled)

20. The composition according to claim 14, wherein the at least one monomer in the first region is a nucleoside analog selected from the group consisting of an LNA monomer, a monomer containing a 2′-O-alkyl-ribose sugar, a monomer containing a 2′-O-methyl-ribose sugar, a monomer containing a 2′-amino-deoxyribose sugar, and a monomer containing a 2′fluoro-deoxyribose sugar.

21-24. (canceled)

25. A method of inhibiting the proliferation of a mammalian cell or tissue, comprising contacting said cell or tissue with:
(a) an effective amount of an oligomer, or a conjugate comprising an oligomer, the oligomer consisting of 10 to 50 contiguous monomers wherein adjacent monomers are covalently linked by a phosphate group or a phosphorothioate group,
wherein said oligomer comprises a first region of at least 10 contiguous monomers;
wherein at least one monomer of said first region is a nucleoside analog;
wherein the sequence of said first region is at least 80% identical to the reverse complement of the best-aligned target region of a mammalian HER3 gene or a mammalian HER3 mRNA; and
(b) an effective amount of a protein tyrosine kinase inhibitor.

26. The method of claim 25, wherein the oligomer consists of the sequence:
(SEQ ID NO: 180)
5′-TₛAₛGₛcₛcₛtₛgₛtₛcₛaₛcₛtₛtₛ^{Me}CₛTₛ^{Me}C-3′,
wherein uppercase letters denote beta-D-oxy-LNA monomers and lowercase letters denote DNA monomers, the subscript “s” denotes a phosphorothioate linkage, and ^{Me}C denotes a beta-D-oxy-LNA monomer containing a 5-methylcytosine base; and
wherein said protein tyrosine kinase inhibitor is gefitinib.

27. The method of claim 25, wherein the proliferation of said cell is inhibited by at least about 30% when compared to the proliferation of an untreated cell of the same type.

28. The method of claim 25, wherein the cell is a cancer cell selected from the group consisting of a prostate cancer cell, a breast cancer cell, a lung cancer cell and an epithelial carcinoma cell.

29-32. (canceled)

33. A method of treating cancer in a mammal, comprising administering to said mammal:
(a) an effective amount of an oligomer consisting of 10 to 50 contiguous monomers wherein adjacent monomers are covalently linked by a phosphate group or a phosphorothioate group,
wherein said oligomer comprises a first region of at least 10 contiguous monomers;
wherein at least one monomer of said first region is a nucleoside analog;
wherein the sequence of said first region is at least 80% identical to the reverse complement of the best-aligned target region of a mammalian HER3 gene or a mammalian HER3 mRNA; and
(b) an effective amount of a protein tyrosine kinase inhibitor.

34. The method of claim 33, wherein said oligomer consists of the sequence:
(SEQ ID NO: 180)
5′-TₛAₛGₛcₛcₛtₛgₛtₛcₛaₛcₛtₛtₛ^{Me}CₛTₛ^{Me}C-3′,
wherein uppercase letters denote beta-D-oxy-LNA monomers and lowercase letters denote DNA monomers, the subscript “s” denotes a phosphorothioate linkage, and ^{Me}C denotes a beta-D-oxy-LNA monomer containing a 5-methylcytosine base; and
wherein said protein tyrosine kinase inhibitor is gefitinib.

35. The method of claim 34, wherein the cancer is selected from the group consisting of non-Hodgkin's lymphoma, Hodgkin's lymphoma, acute leukemia, acute lymphocytic leukemia, acute myelocytic leukemia, chronic myeloid leukemia, chronic lymphocytic leukemia, multiple myeloma, colon carcinoma, rectal carcinoma, epithelial carcinoma, pancreatic cancer, breast cancer, ovarian cancer, prostate cancer, renal cell carcinoma, hepatoma, bile duct carcinoma, choriocarcinoma, cervical cancer, testicular cancer, lung carcinoma, bladder carcinoma, melanoma, head and neck cancer, brain cancer, cancers of unknown primary site, neoplasms, cancers of the peripheral nervous system, cancers of the central nervous system, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumour, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, seminoma, embryonal carcinoma, Wilms' tumour, small cell lung carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, and retinoblastoma.

36. The method of claim 33, wherein said oligomer and said protein tyrosine kinase inhibitor are administered separately.

37. The method of claim 33, wherein said oligomer and said protein tyrosine kinase inhibitor are administered concurrently or simultaneously.

38. The method of claim 33, wherein said oligomer and said protein tyrosine kinase inhibitor are administered sequentially.

39. The method of claim 33, wherein said oligomer and said protein tyrosine kinase inhibitor are in pharmaceutical dosage forms suitable for oral administration.

40. The method of claim 33, wherein said oligomer is in a pharmaceutical dosage form suitable for intravenous administration and said protein tyrosine kinase inhibitor is in a pharmaceutical dosage form suitable for oral administration.

41. The method of claim 35, wherein the cancer is selected from the group consisting of lung cancer, prostate cancer, breast cancer and epithelial carcinoma.

42. The method of claim 33, wherein the mammal is a human.

43-47. (canceled)

48. A kit for use in the treatment of cancer, said kit comprising a protein tyrosine kinase and an LNA oligomer targeting HER3.


SEQUENCE LISTING (255 sequences)

SEQ ID NO: 1 - DNA, 16 bp, Artificial Sequence
  Description of Artificial Sequence Synthetic oligonucleotide
gctccagacatcactc

SEQ ID NO: 2 - DNA, 15 bp, Artificial Sequence
  Description of Artificial Sequence Synthetic oligonucleotide
gctccagacatcact

SEQ ID NO: 3 - DNA, 15 bp, Artificial Sequence
  Description of Artificial Sequence Synthetic oligonucleotide
ctccagacatcactc

SEQ ID NO: 4 - DNA, 14 bp, Artificial Sequence
  Description of Artificial Sequence Synthetic oligonucleotide
gctccagacatcac

SEQ ID NO: 5 - DNA, 14 bp, Artificial Sequence
  Description of Artificial Sequence Synthetic oligonucleotide
ctccagacatcact

SEQ ID NO: 6 - DNA, 14 bp, Artificial Sequence
  Description of Artificial Sequence Synthetic oligonucleotide
tccagacatcactc

SEQ ID NO: 7 - DNA, 13 bp, Artificial Sequence
  Description of Artificial Sequence Synthetic oligonucleotide
gctccagacatca

SEQ ID NO: 8 - DNA, 13 bp, Artificial Sequence
  Description of Artificial Sequence Synthetic oligonucleotide
ctccagacatcac

SEQ ID NO: 9 - DNA, 13 bp, Artificial Sequence
  Description of Artificial Sequence Synthetic oligonucleotide
tccagacatcact

SEQ ID NO: 10 - DNA, 13 bp, Artificial Sequence
  Description of Artificial Sequence Synthetic oligonucleotide
ccagacatcactc

SEQ ID NO: 11 - DNA, 12 bp, Artificial Sequence
  Description of Artificial Sequence Synthetic oligonucleotide
gctccagacatc

SEQ ID NO: 12 - DNA, 12 bp, Artificial Sequence
  Description of Artificial Sequence Synthetic oligonucleotide
ctccagacatca

SEQ ID NO: 13 - DNA, 12 bp, Artificial Sequence
  Description of Artificial Sequence Synthetic oligonucleotide
tccagacatcac

SEQ ID NO: 14 - DNA, 12 bp, Artificial Sequence
  Description of Artificial Sequence Synthetic oligonucleotide
ccagacatcact

SEQ ID NO: 15 - DNA, 12 bp, Artificial Sequence
  Description of Artificial Sequence Synthetic oligonucleotide
cagacatcactc

SEQ ID NO: 16 - DNA, 16 bp, Artificial Sequence
  Description of Artificial Sequence Synthetic oligonucleotide
ctccagacatcactct

SEQ ID NO: 17 - DNA, 16 bp, Artificial Sequence
  Description of Artificial Sequence Synthetic oligonucleotide
cagacatcactctggt

SEQ ID NO: 18 - DNA, 16 bp, Artificial Sequence
  Description of Artificial Sequence Synthetic oligonucleotide
agacatcactctggtg

SEQ ID NO: 19 - DNA, 16 bp, Artificial Sequence
  Description of Artificial Sequence Synthetic oligonucleotide
atagctccagacatca

SEQ ID NO: 20 - DNA, 15 bp, Artificial Sequence
  Description of Artificial Sequence Synthetic oligonucleotide
atagctccagacatc

SEQ ID NO: 21 - DNA, 15 bp, Artificial Sequence
  Description of Artificial Sequence Synthetic oligonucleotide
tagctccagacatca

SEQ ID NO: 22 - DNA, 14 bp, Artificial Sequence
  Description of Artificial Sequence Synthetic oligonucleotide
atagctccagacat

SEQ ID NO: 23 - DNA, 14 bp, Artificial Sequence
  Description of Artificial Sequence Synthetic oligonucleotide
tagctccagacatc

SEQ ID NO: 24 - DNA, 14 bp, Artificial Sequence
  Description of Artificial Sequence Synthetic oligonucleotide
agctccagacatca

SEQ ID NO: 25 - DNA, 13 bp, Artificial Sequence
  Description of Artificial Sequence Synthetic oligonucleotide
atagctccagaca

SEQ ID NO: 26 - DNA, 13 bp, Artificial Sequence
  Description of Artificial Sequence Synthetic oligonucleotide
tagctccagacat

SEQ ID NO: 27 - DNA, 13 bp, Artificial Sequence
  Description of Artificial Sequence Synthetic oligonucleotide
agctccagacatc

SEQ ID NO: 28 - DNA, 13 bp, Artificial Sequence
  Description of Artificial Sequence Synthetic oligonucleotide
gctccagacatca

SEQ ID NO: 29 - DNA, 12 bp, Artificial Sequence
  Description of Artificial Sequence Synthetic oligonucleotide
atagctccagac

SEQ ID NO: 30 - DNA, 12 bp, Artificial Sequence
  Description of Artificial Sequence Synthetic oligonucleotide
tagctccagaca

SEQ ID NO: 31 - DNA, 12 bp, Artificial Sequence
  Description of Artificial Sequence Synthetic oligonucleotide
agctccagacat

SEQ ID NO: 32 - DNA, 12 bp, Artificial Sequence
  Description of Artificial Sequence Synthetic oligonucleotide
gctccagacatc

SEQ ID NO: 33 - DNA, 12 bp, Artificial Sequence
  Description of Artificial Sequence Synthetic oligonucleotide
ctccagacatca

SEQ ID NO: 34 - DNA, 16 bp, Artificial Sequence
  Description of Artificial Sequence Synthetic oligonucleotide
tcacaccatagctcca

SEQ ID NO: 35 - DNA, 15 bp, Artificial Sequence
  Description of Artificial Sequence Synthetic oligonucleotide
tcacaccatagctcc

SEQ ID NO: 36 - DNA, 15 bp, Artificial Sequence
  Description of Artificial Sequence Synthetic oligonucleotide
cacaccatagctcca

SEQ ID NO: 37 - DNA, 14 bp, Artificial Sequence
  Description of Artificial Sequence Synthetic oligonucleotide
tcacaccatagctc

SEQ ID NO: 38 - DNA, 14 bp, Artificial Sequence
  Description of Artificial Sequence Synthetic oligonucleotide
cacaccatagctcc

SEQ ID NO: 39 - DNA, 14 bp, Artificial Sequence
  Description of Artificial Sequence Synthetic oligonucleotide
acaccatagctcca

SEQ ID NO: 40 - DNA, 13 bp, Artificial Sequence
  Description of Artificial Sequence Synthetic oligonucleotide
tcacaccatagct

SEQ ID NO: 41 - DNA, 13 bp, Artificial Sequence
  Description of Artificial Sequence Synthetic oligonucleotide
cacaccatagctc

SEQ ID NO: 42 - DNA, 13 bp, Artificial Sequence
  Description of Artificial Sequence Synthetic oligonucleotide
acaccatagctcc

SEQ ID NO: 43 - DNA, 13 bp, Artificial Sequence
  Description of Artificial Sequence Synthetic oligonucleotide
caccatagctcca

SEQ ID NO: 44 - DNA, 12 bp, Artificial Sequence
  Description of Artificial Sequence Synthetic oligonucleotide
tcacaccatagc

SEQ ID NO: 45 - DNA, 12 bp, Artificial Sequence
  Description of Artificial Sequence Synthetic oligonucleotide
cacaccatagct

SEQ ID NO: 46 - DNA, 12 bp, Artificial Sequence
  Description of Artificial Sequence Synthetic oligonucleotide
acaccatagctc

SEQ ID NO: 47 - DNA, 12 bp, Artificial Sequence
  Description of Artificial Sequence Synthetic oligonucleotide
caccatagctcc

SEQ ID NO: 48 - DNA, 12 bp, Artificial Sequence
  Description of Artificial Sequence Synthetic oligonucleotide
accatagctcca

SEQ ID NO: 49 - DNA, 16 bp, Artificial Sequence
  Description of Artificial Sequence Synthetic oligonucleotide
catccaacacttgacc

SEQ ID NO: 50 - DNA, 16 bp, Artificial Sequence
  Description of Artificial Sequence Synthetic oligonucleotide
atccaacacttgacca

SEQ ID NO: 51 - DNA, 16 bp, Artificial Sequence
  Description of Artificial Sequence Synthetic oligonucleotide
caatcatccaacactt

SEQ ID NO: 52 - DNA, 16 bp, Artificial Sequence
  Description of Artificial Sequence Synthetic oligonucleotide
tcaatcatccaacact

SEQ ID NO: 53 - DNA, 16 bp, Artificial Sequence
  Description of Artificial Sequence Synthetic oligonucleotide
catgtagacatcaatt

SEQ ID NO: 54 - DNA, 16 bp, Artificial Sequence
  Description of Artificial Sequence Synthetic oligonucleotide
tagcctgtcacttctc

SEQ ID NO: 55 - DNA, 16 bp, Artificial Sequence
  Description of Artificial Sequence Synthetic oligonucleotide
agatggcaaacttccc

SEQ ID NO: 56 - DNA, 16 bp, Artificial Sequence
  Description of Artificial Sequence Synthetic oligonucleotide
caaggctcacacatct

SEQ ID NO: 57 - DNA, 16 bp, Artificial Sequence
  Description of Artificial Sequence Synthetic oligonucleotide
aagtccaggttgccca

SEQ ID NO: 58 - DNA, 16 bp, Artificial Sequence
  Description of Artificial Sequence Synthetic oligonucleotide
cattcaagttcttcat

SEQ ID NO: 59 - DNA, 16 bp, Artificial Sequence
  Description of Artificial Sequence Synthetic oligonucleotide
cactaatttccttcag

SEQ ID NO: 60 - DNA, 15 bp, Artificial Sequence
  Description of Artificial Sequence Synthetic oligonucleotide
cactaatttccttca

SEQ ID NO: 61 - DNA, 15 bp, Artificial Sequence
  Description of Artificial Sequence Synthetic oligonucleotide
actaatttccttcag

SEQ ID NO: 62 - DNA, 14 bp, Artificial Sequence
  Description of Artificial Sequence Synthetic oligonucleotide
cactaatttccttc

SEQ ID NO: 63 - DNA, 14 bp, Artificial Sequence
  Description of Artificial Sequence Synthetic oligonucleotide
actaatttccttca

SEQ ID NO: 64 - DNA, 14 bp, Artificial Sequence
  Description of Artificial Sequence Synthetic oligonucleotide
ctaatttccttcag

SEQ ID NO: 65 - DNA, 13 bp, Artificial Sequence
  Description of Artificial Sequence Synthetic oligonucleotide
cactaatttcctt

SEQ ID NO: 66 - DNA, 13 bp, Artificial Sequence
  Description of Artificial Sequence Synthetic oligonucleotide
actaatttccttc

SEQ ID NO: 67 - DNA, 13 bp, Artificial Sequence
  Description of Artificial Sequence Synthetic oligonucleotide
ctaatttccttca

SEQ ID NO: 68 - DNA, 13 bp, Artificial Sequence
  Description of Artificial Sequence Synthetic oligonucleotide
taatttccttcag

SEQ ID NO: 69 - DNA, 12 bp, Artificial Sequence
  Description of Artificial Sequence Synthetic oligonucleotide
cactaatttcct

SEQ ID NO: 70 - DNA, 12 bp, Artificial Sequence
  Description of Artificial Sequence Synthetic oligonucleotide
actaatttcctt

SEQ ID NO: 71 - DNA, 12 bp, Artificial Sequence
  Description of Artificial Sequence Synthetic oligonucleotide
ctaatttccttc

SEQ ID NO: 72 - DNA, 12 bp, Artificial Sequence
  Description of Artificial Sequence Synthetic oligonucleotide
taatttccttca

SEQ ID NO: 73 - DNA, 12 bp, Artificial Sequence
  Description of Artificial Sequence Synthetic oligonucleotide
aatttccttcag

SEQ ID NO: 74 - DNA, 16 bp, Artificial Sequence
  Description of Artificial Sequence Synthetic oligonucleotide
gcccagcactaatttc

SEQ ID NO: 75 - DNA, 16 bp, Artificial Sequence
  Description of Artificial Sequence Synthetic oligonucleotide
ctttgccctctgccac

SEQ ID NO: 76 - DNA, 16 bp, Artificial Sequence
  Description of Artificial Sequence Synthetic oligonucleotide
cacacactttgccctc

SEQ ID NO: 77 - DNA, 15 bp, Artificial Sequence
  Description of Artificial Sequence Synthetic oligonucleotide
cacacactttgccct

SEQ ID NO: 78 - DNA, 15 bp, Artificial Sequence
  Description of Artificial Sequence Synthetic oligonucleotide
acacactttgccctc

SEQ ID NO: 79 - DNA, 14 bp, Artificial Sequence
  Description of Artificial Sequence Synthetic oligonucleotide
cacacactttgccc

SEQ ID NO: 80 - DNA, 14 bp, Artificial Sequence
  Description of Artificial Sequence Synthetic oligonucleotide
acacactttgccct

SEQ ID NO: 81 - DNA, 14 bp, Artificial Sequence
  Description of Artificial Sequence Synthetic oligonucleotide
cacactttgccctc

SEQ ID NO: 82 - DNA, 13 bp, Artificial Sequence
  Description of Artificial Sequence Synthetic oligonucleotide
cacacactttgcc

SEQ ID NO: 83 - DNA, 13 bp, Artificial Sequence
  Description of Artificial Sequence Synthetic oligonucleotide
acacactttgccc

SEQ ID NO: 84 - DNA, 13 bp, Artificial Sequence
  Description of Artificial Sequence Synthetic oligonucleotide
cacactttgccct

SEQ ID NO: 85 - DNA, 13 bp, Artificial Sequence
  Description of Artificial Sequence Synthetic oligonucleotide
acactttgccctc

SEQ ID NO: 86 - DNA, 12 bp, Artificial Sequence
  Description of Artificial Sequence Synthetic oligonucleotide
cacacactttgc

SEQ ID NO: 87 - DNA, 12 bp, Artificial Sequence
  Description of Artificial Sequence Synthetic oligonucleotide
acacactttgcc

SEQ ID NO: 88 - DNA, 12 bp, Artificial Sequence
  Description of Artificial Sequence Synthetic oligonucleotide
cacactttgccc

SEQ ID NO: 89 - DNA, 12 bp, Artificial Sequence
  Description of Artificial Sequence Synthetic oligonucleotide
acactttgccct

SEQ ID NO: 90 - DNA, 12 bp, Artificial Sequence
  Description of Artificial Sequence Synthetic oligonucleotide
cactttgccctc

SEQ ID NO: 91 - DNA, 16 bp, Artificial Sequence
  Description of Artificial Sequence Synthetic oligonucleotide
cagttccaaagacacc

SEQ ID NO: 92 - DNA, 16 bp, Artificial Sequence
  Description of Artificial Sequence Synthetic oligonucleotide
tggcaatttgtactcc

SEQ ID NO: 93 - DNA, 15 bp, Artificial Sequence
  Description of Artificial Sequence Synthetic oligonucleotide
tggcaatttgtactc

SEQ ID NO: 94 - DNA, 15 bp, Artificial Sequence
  Description of Artificial Sequence Synthetic oligonucleotide
ggcaatttgtactcc

SEQ ID NO: 95 - DNA, 14 bp, Artificial Sequence
  Description of Artificial Sequence Synthetic oligonucleotide
tggcaatttgtact

SEQ ID NO: 96 - DNA, 14 bp, Artificial Sequence
  Description of Artificial Sequence Synthetic oligonucleotide
ggcaatttgtactc

SEQ ID NO: 97 - DNA, 14 bp, Artificial Sequence
  Description of Artificial Sequence Synthetic oligonucleotide
gcaatttgtactcc

SEQ ID NO: 98 - DNA, 13 bp, Artificial Sequence
  Description of Artificial Sequence Synthetic oligonucleotide
tggcaatttgtac

SEQ ID NO: 99 - DNA, 13 bp, Artificial Sequence
  Description of Artificial Sequence Synthetic oligonucleotide
ggcaatttgtact

SEQ ID NO: 100 - DNA, 13 bp, Artificial Sequence
  Description of Artificial Sequence Synthetic oligonucleotide
gcaatttgtactc

SEQ ID NO: 101 - DNA, 13 bp, Artificial Sequence
  Description of Artificial Sequence Synthetic oligonucleotide
caatttgtactcc

SEQ ID NO: 102 - DNA, 12 bp, Artificial Sequence
  Description of Artificial Sequence Synthetic oligonucleotide
tggcaatttgta

SEQ ID NO: 103 - DNA, 12 bp, Artificial Sequence
  Description of Artificial Sequence Synthetic oligonucleotide
ggcaatttgtac

SEQ ID NO: 104 - DNA, 12 bp, Artificial Sequence
  Description of Artificial Sequence Synthetic oligonucleotide
gcaatttgtact

SEQ ID NO: 105 - DNA, 12 bp, Artificial Sequence
  Description of Artificial Sequence Synthetic oligonucleotide
caatttgtactc

SEQ ID NO: 106 - DNA, 12 bp, Artificial Sequence
  Description of Artificial Sequence Synthetic oligonucleotide
aatttgtactcc

SEQ ID NO: 107 - DNA, 16 bp, Artificial Sequence
  Description of Artificial Sequence Synthetic oligonucleotide
gtgtgtgtatttccca

SEQ ID NO: 108 - DNA, 15 bp, Artificial Sequence
  Description of Artificial Sequence Synthetic oligonucleotide
gtgtgtgtatttccc

SEQ ID NO: 109 - DNA, 15 bp, Artificial Sequence
  Description of Artificial Sequence Synthetic oligonucleotide
tgtgtgtatttccca

SEQ ID NO: 110 - DNA, 14 bp, Artificial Sequence
  Description of Artificial Sequence Synthetic oligonucleotide
gtgtgtgtatttcc

SEQ ID NO: 111 - DNA, 14 bp, Artificial Sequence
  Description of Artificial Sequence Synthetic oligonucleotide
tgtgtgtatttccc

SEQ ID NO: 112 - DNA, 14 bp, Artificial Sequence
  Description of Artificial Sequence Synthetic oligonucleotide
gtgtgtatttccca

SEQ ID NO: 113 - DNA, 13 bp, Artificial Sequence
  Description of Artificial Sequence Synthetic oligonucleotide
gtgtgtgtatttc

SEQ ID NO: 114 - DNA, 13 bp, Artificial Sequence
  Description of Artificial Sequence Synthetic oligonucleotide
tgtgtgtatttcc

SEQ ID NO: 115 - DNA, 13 bp, Artificial Sequence
  Description of Artificial Sequence Synthetic oligonucleotide
gtgtgtatttccc

SEQ ID NO: 116 - DNA, 13 bp, Artificial Sequence
  Description of Artificial Sequence Synthetic oligonucleotide
tgtgtatttccca

SEQ ID NO: 117 - DNA, 12 bp, Artificial Sequence
  Description of Artificial Sequence Synthetic oligonucleotide
gtgtgtgtattt

SEQ ID NO: 118 - DNA, 12 bp, Artificial Sequence
  Description of Artificial Sequence Synthetic oligonucleotide
tgtgtgtatttc

SEQ ID NO: 119 - DNA, 12 bp, Artificial Sequence
  Description of Artificial Sequence Synthetic oligonucleotide
gtgtgtatttcc

SEQ ID NO: 120 - DNA, 12 bp, Artificial Sequence
  Description of Artificial Sequence Synthetic oligonucleotide
tgtgtatttccc

SEQ ID NO: 121 - DNA, 12 bp, Artificial Sequence
  Description of Artificial Sequence Synthetic oligonucleotide
gtgtatttccca

SEQ ID NO: 122 - DNA, 16 bp, Artificial Sequence
  Description of Artificial Sequence Synthetic oligonucleotide
ccctctgatgactctg

SEQ ID NO: 123 - DNA, 15 bp, Artificial Sequence
  Description of Artificial Sequence Synthetic oligonucleotide
ccctctgatgactct

SEQ ID NO: 124 - DNA, 15 bp, Artificial Sequence
  Description of Artificial Sequence Synthetic oligonucleotide
cctctgatgactctg

SEQ ID NO: 125 - DNA, 14 bp, Artificial Sequence
  Description of Artificial Sequence Synthetic oligonucleotide
ccctctgatgactc

SEQ ID NO: 126 - DNA, 14 bp, Artificial Sequence
  Description of Artificial Sequence Synthetic oligonucleotide
cctctgatgactct

SEQ ID NO: 127 - DNA, 14 bp, Artificial Sequence
  Description of Artificial Sequence Synthetic oligonucleotide
ctctgatgactctg

SEQ ID NO: 128 - DNA, 13 bp, Artificial Sequence
  Description of Artificial Sequence Synthetic oligonucleotide
ccctctgatgact

SEQ ID NO: 129 - DNA, 13 bp, Artificial Sequence
  Description of Artificial Sequence Synthetic oligonucleotide
cctctgatgactc

SEQ ID NO: 130 - DNA, 13 bp, Artificial Sequence
  Description of Artificial Sequence Synthetic oligonucleotide
ctctgatgactct

SEQ ID NO: 131 - DNA, 13 bp, Artificial Sequence
  Description of Artificial Sequence Synthetic oligonucleotide
tctgatgactctg

SEQ ID NO: 132 - DNA, 12 bp, Artificial Sequence
  Description of Artificial Sequence Synthetic oligonucleotide
ccctctgatgac

SEQ ID NO: 133 - DNA, 12 bp, Artificial Sequence
  Description of Artificial Sequence Synthetic oligonucleotide
cctctgatgact

SEQ ID NO: 134 - DNA, 12 bp, Artificial Sequence
  Description of Artificial Sequence Synthetic oligonucleotide
ctctgatgactc

SEQ ID NO: 135 - DNA, 12 bp, Artificial Sequence
  Description of Artificial Sequence Synthetic oligonucleotide
tctgatgactct

SEQ ID NO: 136 - DNA, 12 bp, Artificial Sequence
  Description of Artificial Sequence Synthetic oligonucleotide
ctgatgactctg

SEQ ID NO: 137 - DNA, 16 bp, Artificial Sequence
  Description of Artificial Sequence Synthetic oligonucleotide
catactcctcatcttc

SEQ ID NO: 138 - DNA, 16 bp, Artificial Sequence
  Description of Artificial Sequence Synthetic oligonucleotide
ccaccacaaagttatg

SEQ ID NO: 139 - DNA, 16 bp, Artificial Sequence
  Description of Artificial Sequence Synthetic oligonucleotide
catcactctggtgtgt

SEQ ID NO: 140 - DNA, 16 bp, Artificial Sequence
  Description of Artificial Sequence Synthetic oligonucleotide
gacatcactctggtgt

SEQ ID NO: 141 - DNA, 16 bp, Artificial Sequence
  Description of Artificial Sequence Synthetic oligonucleotide
gctccagacatcactc

SEQ ID NO: 142 - DNA, 16 bp, Artificial Sequence
  Description of Artificial Sequence Synthetic oligonucleotide
ctccagacatcactct

SEQ ID NO: 143 - DNA, 16 bp, Artificial Sequence
  Description of Artificial Sequence Synthetic oligonucleotide
cagacatcactctggt

SEQ ID NO: 144 - DNA, 16 bp, Artificial Sequence
  Description of Artificial Sequence Synthetic oligonucleotide
agacatcactctggtg

SEQ ID NO: 145 - DNA, 16 bp, Artificial Sequence
  Description of Artificial Sequence Synthetic oligonucleotide
atagctccagacatca

SEQ ID NO: 146 - DNA, 16 bp, Artificial Sequence
  Description of Artificial Sequence Synthetic oligonucleotide
tcacaccatagctcca

SEQ ID NO: 147 - DNA, 16 bp, Artificial Sequence
  Description of Artificial Sequence Synthetic oligonucleotide
catccaacacttgacc

SEQ ID NO: 148 - DNA, 16 bp, Artificial Sequence
  Description of Artificial Sequence Synthetic oligonucleotide
atccaacacttgacca

SEQ ID NO: 149 - DNA, 16 bp, Artificial Sequence
  Description of Artificial Sequence Synthetic oligonucleotide
caatcatccaacactt

SEQ ID NO: 150 - DNA, 16 bp, Artificial Sequence
  Description of Artificial Sequence Synthetic oligonucleotide
tcaatcatccaacact

SEQ ID NO: 151 - DNA, 16 bp, Artificial Sequence
  Description of Artificial Sequence Synthetic oligonucleotide
catgtagacatcaatt

SEQ ID NO: 152 - DNA, 16 bp, Artificial Sequence
  Description of Artificial Sequence Synthetic oligonucleotide
tagcctgtcacttctc

SEQ ID NO: 153 - DNA, 16 bp, Artificial Sequence
  Description of Artificial Sequence Synthetic oligonucleotide
agatggcaaacttccc

SEQ ID NO: 154 - DNA, 16 bp, Artificial Sequence
  Description of Artificial Sequence Synthetic oligonucleotide
caaggctcacacatct

SEQ ID NO: 155 - DNA, 16 bp, Artificial Sequence
  Description of Artificial Sequence Synthetic oligonucleotide
aagtccaggttgccca

SEQ ID NO: 156 - DNA, 16 bp, Artificial Sequence
  Description of Artificial Sequence Synthetic oligonucleotide
cattcaagttcttcat

SEQ ID NO: 157 - DNA, 16 bp, Artificial Sequence
  Description of Artificial Sequence Synthetic oligonucleotide
cactaatttccttcag

SEQ ID NO: 158 - DNA, 16 bp, Artificial Sequence
  Description of Artificial Sequence Synthetic oligonucleotide
gcccagcactaatttc

SEQ ID NO: 159 - DNA, 16 bp, Artificial Sequence
  Description of Artificial Sequence Synthetic oligonucleotide
ctttgccctctgccac

SEQ ID NO: 160 - DNA, 16 bp, Artificial Sequence
  Description of Artificial Sequence Synthetic oligonucleotide
cacacactttgccctc

SEQ ID NO: 161 - DNA, 16 bp, Artificial Sequence
  Description of Artificial Sequence Synthetic oligonucleotide
cagttccaaagacacc

SEQ ID NO: 162 - DNA, 16 bp, Artificial Sequence
  Description of Artificial Sequence Synthetic oligonucleotide
tggcaatttgtactcc

SEQ ID NO: 163 - DNA, 16 bp, Artificial Sequence
  Description of Artificial Sequence Synthetic oligonucleotide
gtgtgtgtatttccca

SEQ ID NO: 164 - DNA, 16 bp, Artificial Sequence
  Description of Artificial Sequence Synthetic oligonucleotide
ccctctgatgactctg

SEQ ID NO: 165 - DNA, 16 bp, Artificial Sequence
  Description of Artificial Sequence Synthetic oligonucleotide
catactcctcatcttc

SEQ ID NO: 166 - DNA, 16 bp, Artificial Sequence
  Description of Artificial Sequence Synthetic oligonucleotide
ccaccacaaagttatg

SEQ ID NO: 167 - DNA, 16 bp, Artificial Sequence
  Description of Artificial Sequence Synthetic oligonucleotide
catcactctggtgtgt

SEQ ID NO: 168 - DNA, 16 bp, Artificial Sequence
  Description of Artificial Sequence Synthetic oligonucleotide
gacatcactctggtgt

SEQ ID NO: 169 - DNA, 16 bp, Artificial Sequence
  Description of Artificial Sequence Synthetic oligonucleotide
gctccagacatcactc

SEQ ID NO: 170 - DNA, 16 bp, Artificial Sequence
  Description of Artificial Sequence Synthetic oligonucleotide
ctccagacatcactct

SEQ ID NO: 171 - DNA, 16 bp, Artificial Sequence
  Description of Artificial Sequence Synthetic oligonucleotide
cagacatcactctggt

SEQ ID NO: 172 - DNA, 16 bp, Artificial Sequence
  Description of Artificial Sequence Synthetic oligonucleotide
agacatcactctggtg

SEQ ID NO: 173 - DNA, 16 bp, Artificial Sequence
  Description of Artificial Sequence Synthetic oligonucleotide
atagctccagacatca

SEQ ID NO: 174 - DNA, 16 bp, Artificial Sequence
  Description of Artificial Sequence Synthetic oligonucleotide
tcacaccatagctcca

SEQ ID NO: 175 - DNA, 16 bp, Artificial Sequence
  Description of Artificial Sequence Synthetic oligonucleotide
catccaacacttgacc

SEQ ID NO: 176 - DNA, 16 bp, Artificial Sequence
  Description of Artificial Sequence Synthetic oligonucleotide
atccaacacttgacca

SEQ ID NO: 177 - DNA, 16 bp, Artificial Sequence
  Description of Artificial Sequence Synthetic oligonucleotide
caatcatccaacactt

SEQ ID NO: 178 - DNA, 16 bp, Artificial Sequence
  Description of Artificial Sequence Synthetic oligonucleotide
tcaatcatccaacact

SEQ ID NO: 179 - DNA, 16 bp, Artificial Sequence
  Description of Artificial Sequence Synthetic oligonucleotide
catgtagacatcaatt

SEQ ID NO: 180 - DNA, 16 bp, Artificial Sequence
  Description of Artificial Sequence Synthetic oligonucleotide
tagcctgtcacttctc

SEQ ID NO: 181 - DNA, 16 bp, Artificial Sequence
  Description of Artificial Sequence Synthetic oligonucleotide
agatggcaaacttccc

SEQ ID NO: 182 - DNA, 16 bp, Artificial Sequence
  Description of Artificial Sequence Synthetic oligonucleotide
caaggctcacacatct

SEQ ID NO: 183 - DNA, 16 bp, Artificial Sequence
  Description of Artificial Sequence Synthetic oligonucleotide
aagtccaggttgccca

SEQ ID NO: 184 - DNA, 16 bp, Artificial Sequence
  Description of Artificial Sequence Synthetic oligonucleotide
cattcaagttcttcat

SEQ ID NO: 185 - DNA, 16 bp, Artificial Sequence
  Description of Artificial Sequence Synthetic oligonucleotide
cactaatttccttcag

SEQ ID NO: 186 - DNA, 16 bp, Artificial Sequence
  Description of Artificial Sequence Synthetic oligonucleotide
gcccagcactaatttc

SEQ ID NO: 187 - DNA, 16 bp, Artificial Sequence
  Description of Artificial Sequence Synthetic oligonucleotide
ctttgccctctgccac

SEQ ID NO: 188 - DNA, 16 bp, Artificial Sequence
  Description of Artificial Sequence Synthetic oligonucleotide
cacacactttgccctc

SEQ ID NO: 189 - DNA, 16 bp, Artificial Sequence
  Description of Artificial Sequence Synthetic oligonucleotide
cagttccaaagacacc

SEQ ID NO: 190 - DNA, 16 bp, Artificial Sequence
  Description of Artificial Sequence Synthetic oligonucleotide
tggcaatttgtactcc

SEQ ID NO: 191 - DNA, 16 bp, Artificial Sequence
  Description of Artificial Sequence Synthetic oligonucleotide
gtgtgtgtatttccca

SEQ ID NO: 192 - DNA, 16 bp, Artificial Sequence
  Description of Artificial Sequence Synthetic oligonucleotide
ccctctgatgactctg

SEQ ID NO: 193 - DNA, 16 bp, Artificial Sequence
  Description of Artificial Sequence Synthetic oligonucleotide
catactcctcatcttc

SEQ ID NO: 194 - DNA, 16 bp, Artificial Sequence
  Description of Artificial Sequence Synthetic oligonucleotide
ccaccacaaagttatg

SEQ ID NO: 195 - DNA, 16 bp, Artificial Sequence
  Description of Artificial Sequence Synthetic oligonucleotide
catcactctggtgtgt

SEQ ID NO: 196 - DNA, 16 bp, Artificial Sequence
  Description of Artificial Sequence Synthetic oligonucleotide
gacatcactctggtgt

SEQ ID NO: 197 - DNA, 5511 bp, homo sapiens
acacacacacacccctcccctgccatccctccccggactccggctccggctccgattgcaatttgcaacctccgctgccgtcgccgcagcagccaccaattcgccagcggttcaggtggctcttgcctcgatgtcctagcctaggggcccccgggccggacttggctgggctcccttcaccctctgcggagtcatgagggcgaacgacgctctgcaggtgctgggcttgcttttcagcctggcccggggctccgaggtgggcaactctcaggcagtgtgtcctgggactctgaatggcctgagtgtgaccggcgatgctgagaaccaataccagacactgtacaagctctacgagaggtgtgaggtggtgatggggaaccttgagattgtgctcacgggacacaatgccgacctctccttcctgcagtggattcgagaagtgacaggctatgtcctcgtggccatgaatgaattctctactctaccattgcccaacctccgcgtggtgcgagggacccaggtctacgatgggaagtttgccatcttcgtcatgttgaactataacaccaactccagccacgctctgcgccagctccgcttgactcagctcaccgagattctgtcagggggtgtttatattgagaagaacgataagctttgtcacatggacacaattgactggagggacatcgtgagggaccgagatgctgagatagtggtgaaggacaatggcagaagctgtcccccctgtcatgaggtttgcaaggggcgatgctggggtcctggatcagaagactgccagacattgaccaagaccatctgtgctcctcagtgtaatggtcactgctttgggcccaaccccaaccagtgctgccatgatgagtgtgccgggggctgctcaggccctcaggacacagactgctttgcctgccggcacttcaatgacagtggagcctgtgtacctcgctgtccacagcctcttgtctacaacaagctaactttccagctggaacccaatccccacaccaagtatcagtatggaggagtttgtgtagccagctgtccccataactttgtggtggatcaaacatcctgtgtcagggcctgtcctcctgacaagatggaagtagataaaaatgggctcaagatgtgtgagccttgtgggggactatgtcccaaagcctgtgagggaacaggctctgggagccgcttccagactgtggactcgagcaacattgatggatttgtgaactgcaccaagatcctgggcaacctggactttctgatcaccggcctcaatggagacccctggcacaagatccctgccctggacccagagaagctcaatgtcttccggacagtacgggagatcacaggttacctgaacatccagtcctggccgccccacatgcacaacttcagtgttttttccaatttgacaaccattggaggcagaagcctctacaaccggggcttctcattgttgatcatgaagaacttgaatgtcacatctctgggcttccgatccctgaaggaaattagtgctgggcgtatctatataagtgccaataggcagctctgctaccaccactctttgaactggaccaaggtgcttcgggggcctacggaagagcgactagacatcaagcataatcggccgcgcagagactgcgtggcagagggcaaagtgtgtgacccactgtgctcctctgggggatgctggggcccaggccctggtcagtgcttgtcctgtcgaaattatagccgaggaggtgtctgtgtgacccactgcaactttctgaatggggagcctcgagaatttgcccatgaggccgaatgcttctcctgccacccggaatgccaacccatggagggcactgccacatgcaatggctcgggctctgatacttgtgctcaatgtgcccattttcgagatgggccccactgtgtgagcagctgcccccatggagtcctaggtgccaagggcccaatctacaagtacccagatgttcagaatgaatgtcggccctgccatgagaactgcacccaggggtgtaaaggaccagagcttcaagactgtttaggacaaacactggtgctgatcggcaaaacccatctgacaatggctttgacagtgatagcaggattggtagtgattttcatgatgctgggcggcacttttctctactggcgtgggcgccggattcagaataaaagggctatgaggcgatacttggaacggggtgagagcatagagcctctggaccccagtgagaaggctaacaaagtcttggccagaatcttcaaagagacagagctaaggaagcttaaagtgcttggctcgggtgtctttggaactgtgcacaaaggagtgtggatccctgagggtgaatcaatcaagattccagtctgcattaaagtcattgaggacaagagtggacggcagagttttcaagctgtgacagatcatatgctggccattggcagcctggaccatgcccacattgtaaggctgctgggactatgcccagggtcatctctgcagcttgtcactcaatatttgcctctgggttctctgctggatcatgtgagacaacaccggggggcactggggccacagctgctgctcaactggggagtacaaattgccaagggaatgtactaccttgaggaacatggtatggtgcatagaaacctggctgcccgaaacgtgctactcaagtcacccagtcaggttcaggtggcagattttggtgtggctgacctgctgcctcctgatgataagcagctgctatacagtgaggccaagactccaattaagtggatggcccttgagagtatccactttgggaaatacacacaccagagtgatgtctggagctatggtgtgacagtttgggagttgatgaccttcggggcagagccctatgcagggctacgattggctgaagtaccagacctgctagagaagggggagcggttggcacagccccagatctgcacaattgatgtctacatggtgatggtcaagtgttggatgattgatgagaacattcgcccaacctttaaagaactagccaatgagttcaccaggatggcccgagacccaccacggtatctggtcataaagagagagagtgggcctggaatagcccctgggccagagccccatggtctgacaaacaagaagctagaggaagtagagctggagccagaactagacctagacctagacttggaagcagaggaggacaacctggcaaccaccacactgggctccgccctcagcctaccagttggaacacttaatcggccacgtgggagccagagccttttaagtccatcatctggatacatgcccatgaaccagggtaatcttggggagtcttgccaggagtctgcagtttctgggagcagtgaacggtgcccccgtccagtctctctacacccaatgccacggggatgcctggcatcagagtcatcagaggggcatgtaacaggctctgaggctgagctccaggagaaagtgtcaatgtgtaggagccggagcaggagccggagcccacggccacgcggagatagcgcctaccattcccagcgccacagtctgctgactcctgttaccccactctccccacccgggttagaggaagaggatgtcaacggttatgtcatgccagatacacacctcaaaggtactccctcctcccgggaaggcaccctttcttcagtgggtctcagttctgtcctgggtactgaagaagaagatgaagatgaggagtatgaatacatgaaccggaggagaaggcacagtccacctcatccccctaggccaagttcccttgaggagctgggttatgagtacatggatgtggggtcagacctcagtgcctctctgggcagcacacagagttgcccactccaccctgtacccatcatgcccactgcaggcacaactccagatgaagactatgaatatatgaatcggcaacgagatggaggtggtcctgggggtgattatgcagccatgggggcctgcccagcatctgagcaagggtatgaagagatgagagcttttcaggggcctggacatcaggccccccatgtccattatgcccgcctaaaaactctacgtagcttagaggctacagactctgcctttgataaccctgattactggcatagcaggcttttccccaaggctaatgcccagagaacgtaactcctgctccctgtggcactcagggagcatttaatggcagctagtgcctttagagggtaccgtcttctccctattccctctctctcccaggtcccagccccttttccccagtcccagacaattccattcaatctttggaggcttttaaacattttgacacaaaattcttatggtatgtagccagctgtgcactttcttctctttcccaaccccaggaaaggttttccttattttgtgtgctttcccagtcccattcctcagcttcttcacaggcactcctggagatatgaaggattactctccatatcccttcctctcaggctcttgactacttggaactaggctcttatgtgtgcctttgtttcccatcagactgtcaagaagaggaaagggaggaaacctagcagaggaaagtgtaattttggtttatgactcttaaccccctagaaagacagaagcttaaaatctgtgaagaaagaggttaggagtagatattgattactatcataattcagcacttaactatgagccaggcatcatactaaacttcacctacattatctcacttagtcctttatcatccttaaaacaattctgtgacatacatattatctcattttacacaaagggaagtcgggcatggtggctcatgcctgtaatctcagcactttgggaggctgaggcagaaggattacctgaggcaaggagtttgagaccagcttagccaacatagtaagacccccatctctttaaaaaaaaaaaaaaaaaaaaaaaaaaaactttagaactgggtgcagtggctcatgcctgtaatcccagccagcactttgggaggctgagatgggaagatcacttgagcccagaattagagataagcctatggaaacatagcaagacactgtctctacaggggaaaaaaaaaaaagaaactgagccttaaagagatgaaataaattaagcagtagatccaggatgcaaaatcctcccaattcctgtgcatgtgctcttattgtaaggtgccaagaaaaactgatttaagttacagcccttgtttaaggggcactgtttcttgtttttgcactgaatcaagtctaaccccaacagccacatcctcctatacctagacatctcatctcaggaagtggtggtgggggtagtcagaaggaaaaataactggacatctttgtgtaaaccataatccacatgtgccgtaaatgatcttcactccttatccgagggcaaattcacaaggatccccaagatccacttttagaagccattctcatcca

SEQ ID NO: 198 - DNA, 5616 bp, homo sapiens
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 199 - DNA, 4624 bp, homo sapiens
ggaggaggtggaggaggagggctgcttgaggaagtataagaatgaagttgtgaagctgagattcccctccattgggaccggagaaaccaggggagccccccgggcagccgcgcgccccttcccacggggccctttactgcgccgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagcta

SEQ ID NO: 200 - DNA, 24 bp, Artificial Sequence
  Description of Artificial Sequence Synthetic oligonucleotide
catagctccagacatcactctggt

SEQ ID NO: 201 - DNA, 24 bp, Artificial Sequence
  Description of Artificial Sequence Synthetic oligonucleotide
atagctccagacatcactctggtg

SEQ ID NO: 202 - DNA, 24 bp, Artificial Sequence
  Description of Artificial Sequence Synthetic oligonucleotide
gctccagacatcactctggtgtgt

SEQ ID NO: 203 - DNA, 24 bp, Artificial Sequence
  Description of Artificial Sequence Synthetic oligonucleotide
ctccagacatcactctggtgtgtg

SEQ ID NO: 204 - DNA, 24 bp, Artificial Sequence
  Description of Artificial Sequence Synthetic oligonucleotide
caccatagctccagacatcactct

SEQ ID NO: 205 - DNA, 24 bp, Artificial Sequence
  Description of Artificial Sequence Synthetic oligonucleotide
actgtcacaccatagctccagaca

SEQ ID NO: 206 - DNA, 24 bp, Artificial Sequence
  Description of Artificial Sequence Synthetic oligonucleotide
caatcatccaacacttgaccatca

SEQ ID NO: 207 - DNA, 24 bp, Artificial Sequence
  Description of Artificial Sequence Synthetic oligonucleotide
aatcatccaacacttgaccatcac

SEQ ID NO: 208 - DNA, 24 bp, Artificial Sequence
  Description of Artificial Sequence Synthetic oligonucleotide
tcatcaatcatccaacacttgacc

SEQ ID NO: 209 - DNA, 24 bp, Artificial Sequence
  Description of Artificial Sequence Synthetic oligonucleotide
ctcatcaatcatccaacacttgac

SEQ ID NO: 210 - DNA, 24 bp, Artificial Sequence
  Description of Artificial Sequence Synthetic oligonucleotide
tcaccatgtagacatcaattgtgc

SEQ ID NO: 211 - DNA, 24 bp, Artificial Sequence
  Description of Artificial Sequence Synthetic oligonucleotide
gacatagcctgtcacttctcgaat

SEQ ID NO: 212 - DNA, 24 bp, Artificial Sequence
  Description of Artificial Sequence Synthetic oligonucleotide
acgaagatggcaaacttcccatcg

SEQ ID NO: 213 - DNA, 24 bp, Artificial Sequence
  Description of Artificial Sequence Synthetic oligonucleotide
cccacaaggctcacacatcttgag

SEQ ID NO: 214 - DNA, 24 bp, Artificial Sequence
  Description of Artificial Sequence Synthetic oligonucleotide
cagaaagtccaggttgcccaggat

SEQ ID NO: 215 - DNA, 24 bp, Artificial Sequence
  Description of Artificial Sequence Synthetic oligonucleotide
gtgacattcaagttcttcatgatc

SEQ ID NO: 216 - DNA, 24 bp, Artificial Sequence
  Description of Artificial Sequence Synthetic oligonucleotide
ccagcactaatttccttcagggat

SEQ ID NO: 217 - DNA, 24 bp, Artificial Sequence
  Description of Artificial Sequence Synthetic oligonucleotide
atacgcccagcactaatttccttc

SEQ ID NO: 218 - DNA, 24 bp, Artificial Sequence
  Description of Artificial Sequence Synthetic oligonucleotide
cacactttgccctctgccacgcag

SEQ ID NO: 219 - DNA, 24 bp, Artificial Sequence
  Description of Artificial Sequence Synthetic oligonucleotide
gggtcacacactttgccctctgcc

SEQ ID NO: 220 - DNA, 24 bp, Artificial Sequence
  Description of Artificial Sequence Synthetic oligonucleotide
tgcacagttccaaagacacccgag

SEQ ID NO: 221 - DNA, 24 bp, Artificial Sequence
  Description of Artificial Sequence Synthetic oligonucleotide
cccttggcaatttgtactccccag

SEQ ID NO: 222 - DNA, 24 bp, Artificial Sequence
  Description of Artificial Sequence Synthetic oligonucleotide
tctggtgtgtgtatttcccaaagt

SEQ ID NO: 223 - DNA, 24 bp, Artificial Sequence
  Description of Artificial Sequence Synthetic oligonucleotide
atgcccctctgatgactctgatgc

SEQ ID NO: 224 - DNA, 24 bp, Artificial Sequence
  Description of Artificial Sequence Synthetic oligonucleotide
tattcatactcctcatcttcatct

SEQ ID NO: 225 - DNA, 24 bp, Artificial Sequence
  Description of Artificial Sequence Synthetic oligonucleotide
tgatccaccacaaagttatgggga

SEQ ID NO: 226 - DNA, 24 bp, Artificial Sequence
  Description of Artificial Sequence Synthetic oligonucleotide
cagacatcactctggtgtgtgtat

SEQ ID NO: 227 - DNA, 24 bp, Artificial Sequence
  Description of Artificial Sequence Synthetic oligonucleotide
tccagacatcactctggtgtgtgt

SEQ ID NO: 228 - DNA, 15 bp, Artificial Sequence
  Description of Artificial Sequence Synthetic oligonucleotide
tagcctgtcacttct

SEQ ID NO: 229 - DNA, 15 bp, Artificial Sequence
  Description of Artificial Sequence Synthetic oligonucleotide
agcctgtcacttctc

SEQ ID NO: 230 - DNA, 14 bp, Artificial Sequence
  Description of Artificial Sequence Synthetic oligonucleotide
tagcctgtcacttc

SEQ ID NO: 231 - DNA, 14 bp, Artificial Sequence
  Description of Artificial Sequence Synthetic oligonucleotide
agcctgtcacttct

SEQ ID NO: 232 - DNA, 13 bp, Artificial Sequence
  Description of Artificial Sequence Synthetic oligonucleotide
tagcctgtcactt

SEQ ID NO: 233 - DNA, 12 bp, Artificial Sequence
  Description of Artificial Sequence Synthetic oligonucleotide
tagcctgtcact

SEQ ID NO: 234 - DNA, 13 bp, Artificial Sequence
  Description of Artificial Sequence Synthetic oligonucleotide
tagcctgtcactt

SEQ ID NO: 235 - DNA, 16 bp, Artificial
  LNA oligomers
cgtcagtatgcgaatc

SEQ ID NO: 236 - DNA, 16 bp, Artificial
  LNA oligomers
cgcagattagaaacct

SEQ ID NO: 237 - DNA, 22 bp, Artificial
  probe sequence
ccacacctggtcatagcggtga

SEQ ID NO: 238 - DNA, 20 bp, Artificial
  probe sequence
ctgtttaggccaagcagagg

SEQ ID NO: 239 - DNA, 20 bp, Artificial
  probe sequence
attctgaatcctgcgtccac

SEQ ID NO: 240 - DNA, 20 bp, Artificial
  probe sequence
cattgcccaacctccgcgtg

SEQ ID NO: 241 - DNA, 20 bp, Artificial
  probe sequence
tgcagtggattcgagaagtg

SEQ ID NO: 242 - DNA, 20 bp, Artificial
  probe sequence
ggcaaacttcccatcgtaga

SEQ ID NO: 243 - DNA, 20 bp, Artificial
  probe sequence
actggcgctgccaaggctgt

SEQ ID NO: 244 - DNA, 20 bp, Artificial
  probe sequence
ccacccagaagactgtggat

SEQ ID NO: 245 - DNA, 20 bp, Artificial
  probe sequence
ttcagctcagggatgacctt

SEQ ID NO: 246 - DNA, 20 bp, Artificial
  probe sequence
agctgtggcgtgatggccgt

SEQ ID NO: 247 - DNA, 20 bp, Artificial
  probe sequence
aactttggcattgtggaagg

SEQ ID NO: 248 - DNA, 20 bp, Artificial
  probe sequence
ggatgcagggatgatgttct

SEQ ID NO: 249 - DNA, 16 bp, Artificial
  LNA oligomers
tagcctttgacctctc

SEQ ID NO: 250 - DNA, 20 bp, Artificial Sequence
  Description of Artificial Sequence Synthetic probe
cattgcccaacctccgcgtg

SEQ ID NO: 251 - DNA, 20 bp, Artificial Sequence
  Description of Artificial Sequence Synthetic primer
tgcagtggattcgagaagtg

SEQ ID NO: 252 - DNA, 20 bp, Artificial Sequence
  Description of Artificial Sequence Synthetic primer
ggcaaacttcccatcgtaga

SEQ ID NO: 253 - DNA, 20 bp, Artificial Sequence
  Description of Artificial Sequence Synthetic probe
actggcgctgccaaggctgt

SEQ ID NO: 254 - DNA, 20 bp, Artificial Sequence
  Description of Artificial Sequence Synthetic primer
ccacccagaagactgtggat

SEQ ID NO: 255 - DNA, 20 bp, Artificial Sequence
  Description of Artificial Sequence Synthetic primer
ttcagctcagggatgacctt